SEQUENCE LISTING (246 sequences)

SEQ ID NO: 1 - DNA, 3633 bp, unknown
  Sequence of EGFR Exon
atgcgaccctccgggacggccggggcagcgctcctggcgctgctggctgcgctctgcccggcgagtcgggctctggaggaaaagaaagtttgccaaggcacgagtaacaagctcacgcagttgggcacttttgaagatcattttctcagcctccagaggatgttcaataactgtgaggtggtccttgggaatttggaaattacctatgtgcagaggaattatgatctttccttcttaaagaccatccaggaggtggctggttatgtcctcattgccctcaacacagtggagcgaattcctttggaaaacctgcagatcatcagaggaaatatgtactacgaaaattcctatgccttagcagtcttatctaactatgatgcaaataaaaccggactgaaggagctgcccatgagaaatttacaggaaatcctgcatggcgccgtgcggttcagcaacaaccctgccctgtgcaacgtggagagcatccagtggcgggacatagtcagcagtgactttctcagcaacatgtcgatggacttccagaaccacctgggcagctgccaaaagtgtgatccaagctgtcccaatgggagctgctggggtgcaggagaggagaactgccagaaactgaccaaaatcatctgtgcccagcagtgctccgggcgctgccgtggcaagtcccccagtgactgctgccacaaccagtgtgctgcaggctgcacaggcccccgggagagcgactgcctggtctgccgcaaattccgagacgaagccacgtgcaaggacacctgccccccactcatgctctacaaccccaccacgtaccagatggatgtgaaccccgagggcaaatacagctttggtgccacctgcgtgaagaagtgtccccgtaattatgtggtgacagatcacggctcgtgcgtccgagcctgtggggccgacagctatgagatggaggaagacggcgtccgcaagtgtaagaagtgcgaagggccttgccgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaagcttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttcatgcgaaggcgccacatcgttcggaagcgcacgctgcggaggctgctgcaggagagggagcttgtggagcctcttacacccagtggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggcttcttcagcagcccctccacgtcacggactcccctcctgagctctctgagtgcaaccagcaacaattccaccgtggcttgcattgatagaaatgggctgcaaagctgtcccatcaaggaagacagcttcttgcagcgatacagctcagaccccacaggcgccttgactgaggacagcatagacgacaccttcctcccagtgcctgaatacataaaccagtccgttcccaaaaggcccgctggctctgtgcagaatcctgtctatcacaatcagcctctgaaccccgcgcccagcagagacccacactaccaggacccccacagcactgcagtgggcaaccccgagtatctcaacactgtccagcccacctgtgtcaacagcacattcgacagccctgcccactgggcccagaaaggcagccaccaaattagcctggacaaccctgactaccagcaggacttctttcccaaggaagccaagccaaatggcatctttaagggctccacagctgaaaatgcagaatacctaagggtcgcgccacaaagcagtgaatttattggagcatga

SEQ ID NO: 2 - DNA, 20 bp, Artificial
  upstream primer for EGFR Exon 18
gaggatcttgaaggaaactg

SEQ ID NO: 3 - DNA, 19 bp, Artificial
  upstream primer for EGFR Exon 18
ccagcttgtggagcctctt

SEQ ID NO: 4 - DNA, 19 bp, Artificial
  downstream primer for EGFR Exon 18
gccagggaccttaccttat

SEQ ID NO: 5 - DNA, 21 bp, Artificial
  downstream primer for EGFR Exon 18
ctgtgccagggaccttacctt

SEQ ID NO: 6 - DNA, 19 bp, Artificial
  upstream primer for EGFR Exon 19
cccagaaggtgagaaagtt

SEQ ID NO: 7 - DNA, 20 bp, Artificial
  upstream primer for EGFR Exon 19
gggactctggatcccagaag

SEQ ID NO: 8 - DNA, 18 bp, Artificial
  downstream primer for EGFR Exon 19
cctgaggttcagagccat

SEQ ID NO: 9 - DNA, 19 bp, Artificial
  downstream primer for EGFR Exon 19
cccacacagcaaagcagaa

SEQ ID NO: 10 - DNA, 20 bp, Artificial
  upstream primer for EGFR Exon 21
gcagccaggaacgtactggt

SEQ ID NO: 11 - DNA, 20 bp, Artificial
  upstream primer for EGFR Exon 21
ccctcacagcagggtcttct

SEQ ID NO: 12 - DNA, 18 bp, Artificial
  downstream primer for EGFR Exon 21
gtgggaaggcagcctggt

SEQ ID NO: 13 - DNA, 18 bp, Artificial
  downstream primer for EGFR Exon 21
gtgggaaggcagcctggt

SEQ ID NO: 14 - DNA, 21 bp, Artificial
  upstream primer for EGFR Exon 18 position 2155 G->A mutant
tgctgagctccggtgcgttcg

SEQ ID NO: 15 - DNA, 21 bp, Artificial
  downstream primer for EGFR Exon 18 position 2155 G->A mutant
ggagctcagcactttgatctt

SEQ ID NO: 16 - DNA, 21 bp, Artificial
  upstream primer for EGFR Exon 19 position 2235-2249 deletion
atcaaaacatctccgaaagcc

SEQ ID NO: 17 - DNA, 21 bp, Artificial
  downstream primer for EGFR Exon 19 position 2235-2249 deletion
atgttttgatagcgacgggaa

SEQ ID NO: 18 - DNA, 21 bp, Artificial
  upstream primer for EGFR Exon 19 position 2236-2250 deletion
tcaagacatctccgaaagcca

SEQ ID NO: 19 - DNA, 21 bp, Artificial
  downstream primer for EGFR Exon 19 position 2236-2250 deletion
gatgtcttgatagcgacggga

SEQ ID NO: 20 - DNA, 21 bp, Artificial
  upstream primer for EGFR Exon 19 position 2254-2277 deletion
caacactcgatgtgagtttct

SEQ ID NO: 21 - DNA, 21 bp, Artificial
  downstream primer for EGFR Exon 19 position 2254-2277 deletion
tcgagtgttgcttctcttaat

SEQ ID NO: 22 - DNA, 21 bp, Artificial
  upstream primer for EGFR Exon 21 position 2573 T->G mutant
tgggcgggccaaactgctggg

SEQ ID NO: 23 - DNA, 21 bp, Artificial
  downstream primer for EGFR Exon 21 position 2573 T->G mutant
tggcccgcccaaaatctgtga

SEQ ID NO: 24 - DNA, 290 bp, unknown
  Sequence of KRAS gene Exon 1 and 2
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatag

SEQ ID NO: 25 - DNA, 21 bp, Artificial
  upstream primer for KRAS
cctctattgttggatcatatt

SEQ ID NO: 26 - DNA, 27 bp, Artificial
  upstream primer for KRAS
aatgactgaatataaacttgtggtagt

SEQ ID NO: 27 - DNA, 22 bp, Artificial
  downstream primer for KRAS
tgactgaatataaacttgtggt

SEQ ID NO: 28 - DNA, 26 bp, Artificial
  downstream primer for KRAS
aaatgattctgaattagctgtatcgt

SEQ ID NO: 29 - DNA, 21 bp, Artificial
  upstream primer for KRAS Codon 12 GGT->GTT mutant
agctgttggcgtaggcaagag

SEQ ID NO: 30 - DNA, 21 bp, Artificial
  downstream primer for KRAS Codon 12 GGT->GTT mutant
cgccaacagctccaactacca

SEQ ID NO: 31 - DNA, 21 bp, Artificial
  upstream primer for KRAS Codon 12 GGT->AGT mutant
agctagtggcgtaggcaagag

SEQ ID NO: 32 - DNA, 21 bp, Artificial
  downstream primer for KRAS Codon 12 GGT->AGT mutant
cgccactagctccaactacca

SEQ ID NO: 33 - DNA, 21 bp, Artificial
  upstream primer for KRAS Codon 12 GGT->GAT mutant
agctgatggcgtaggcaagag

SEQ ID NO: 34 - DNA, 21 bp, Artificial
  downstream primer for KRAS Codon 12 GGT->GAT mutant
cgccatcagctccaactacca

SEQ ID NO: 35 - DNA, 21 bp, Artificial
  upstream primer for KRAS Codon 12 GGT->TGT mutant
agcttgtggcgtaggcaagag

SEQ ID NO: 36 - DNA, 21 bp, Artificial
  downstream primer for KRAS Codon 12 GGT->TGT mutant
cgccacaagctccaactacca

SEQ ID NO: 37 - DNA, 21 bp, Artificial
  upstream primer for KRAS Codon 13 GGC->GAC mutant
ctggtgacgtaggcaagagtg

SEQ ID NO: 38 - DNA, 21 bp, Artificial
  downstream primer for KRAS Codon 13 GGC->GAC mutant
cctacgtcaccagctccaact

SEQ ID NO: 39 - DNA, 125 bp, unknown
  Sequence of BCRP gene Exon 12
acatgaatacatcagcggatactacagagtgtcatcttatttccttggaaaactgttatctgatttattacccatgaggatgttaccaagtattatatttacctgtatagtgtacttcatgttag

SEQ ID NO: 40 - DNA, 24 bp, Artificial
  upstream primer for BCRP
ctacagagtgtcatcttatttcct

SEQ ID NO: 41 - DNA, 23 bp, Artificial
  upstream primer for BCRP
tccttggaaaactgttatctgat

SEQ ID NO: 42 - DNA, 21 bp, Artificial
  upstream primer for BCRP
gcggatactacagagtgtcat

SEQ ID NO: 43 - DNA, 26 bp, Artificial
  downstream primer for BCRP
catgaagtacactatacaggtaaata

SEQ ID NO: 44 - DNA, 25 bp, Artificial
  downstream primer for BCRP
taacatgaagtacactatacaggta

SEQ ID NO: 45 - DNA, 21 bp, Artificial
  downstream primer for BCRP
ggcaagactaaagacatgtcc

SEQ ID NO: 46 - DNA, 21 bp, Artificial
  upstream primer for BCRP Codon 482 AGG->GGG mutant
ccatggggatgttaccaagta

SEQ ID NO: 47 - DNA, 21 bp, Artificial
  downstream primer for BCRP Codon 482 AGG->GGG mutant
catccccatgggtaataaatc

SEQ ID NO: 48 - DNA, 21 bp, Artificial
  upstream primer for BCRP Codon 482 AGG->ACG mutant
ccatgacgatgttaccaagta

SEQ ID NO: 49 - DNA, 21 bp, Artificial
  downstream primer for BCRP Codon 482 AGG->ACG mutant
catcgtcatgggtaataaatc

SEQ ID NO: 50 - DNA, 100 bp, unknown
  Sequence of BRAF gene Exon 15
ccatgaagacctcacagtagtaaaaataggtgattttggtctagctacagtgaaatctcgatggagtgggtcccatcagtttgaacagttgtctggatcc

SEQ ID NO: 51 - DNA, 30 bp, Artificial
  upstream primer for BRAF
catgaagacctcacagtaaaaataggtgat

SEQ ID NO: 52 - DNA, 24 bp, Artificial
  upstream primer for BRAF
ttcttcatgaagacctcacagtaa

SEQ ID NO: 53 - DNA, 25 bp, Artificial
  downstream primer for BRAF
ggatccagacaactgttcaaactga

SEQ ID NO: 54 - DNA, 23 bp, Artificial
  downstream primer for BRAF
ccagacaactgttcaaactgatg

SEQ ID NO: 55 - DNA, 21 bp, Artificial
  upstream primer for BRAF Codon 600 GTG->GAG mutant
tacagagaaatctcgatggag

SEQ ID NO: 56 - DNA, 21 bp, Artificial
  downstream primer for BRAF Codon 600 GTG->GAG mutant
atttctctgtagctagaccaa

SEQ ID NO: 57 - DNA, 21 bp, Artificial
  upstream primer for detecting ERCC1 expression amount
gaaagagacggagctgaggaa

SEQ ID NO: 58 - DNA, 21 bp, Artificial
  upstream primer for detecting ERCC1 expression amount
gggaatttggcgacgtaattc

SEQ ID NO: 59 - DNA, 21 bp, Artificial
  downstream primer for detecting ERCC1 expression amount
ggccctgaccttgtagactgt

SEQ ID NO: 60 - DNA, 18 bp, Artificial
  downstream primer for detecting ERCC1 expression amount
gcggaggctgaggaacag

SEQ ID NO: 61 - DNA, 24 bp, Artificial
  upstream primer for detecting RRM1 expression amount
tgactactaagcaccctgactatg

SEQ ID NO: 62 - DNA, 16 bp, Artificial
  upstream primer for detecting RRM1 expression amount
acccaccagtcaaagc

SEQ ID NO: 63 - DNA, 24 bp, Artificial
  downstream primer for detecting RRM1 expression amount
cttccatcacatcactgaacactt

SEQ ID NO: 64 - DNA, 20 bp, Artificial
  downstream primer for detecting RRM1 expression amount
catacagggagtggttaagt

SEQ ID NO: 65 - DNA, 31 bp, Artificial
  upstream primer for detecting BRCA1 expression amount
tcatccaaagtatgggctacagatgactatg

SEQ ID NO: 66 - DNA, 23 bp, Artificial
  upstream primer for detecting BRCA1 expression amount
tcatccaaagtatgggctacaga

SEQ ID NO: 67 - DNA, 21 bp, Artificial
  downstream primer for detecting BRCA1 expression amount
tggacactgagactggtttcc

SEQ ID NO: 68 - DNA, 20 bp, Artificial
  downstream primer for detecting BRCA1 expression amount
tggacactgagactggtttc

SEQ ID NO: 69 - DNA, 21 bp, Artificial
  upstream primer for detecting TUBB3 expression amount
gaccgcatctctgtgtactac

SEQ ID NO: 70 - DNA, 22 bp, Artificial
  upstream primer for detecting TUBB3 expression amount
ctgtgtactacaatgaagccac

SEQ ID NO: 71 - DNA, 20 bp, Artificial
  downstream primer for detecting TUBB3 expression amount
gtccatggtcccaggttcta

SEQ ID NO: 72 - DNA, 23 bp, Artificial
  downstream primer for detecting TUBB3 expression amount
aggttctagatccaccaggatgg

SEQ ID NO: 73 - DNA, 21 bp, Artificial
  upstream primer for detecting ERBB3 expression amount
caacggttatgtcatgccaga

SEQ ID NO: 74 - DNA, 22 bp, Artificial
  upstream primer for detecting ERBB3 expression amount
ggttatgtcatgccagatacac

SEQ ID NO: 75 - DNA, 23 bp, Artificial
  downstream primer for detecting ERBB3 expression amount
gacagaactgagacccactgaag

SEQ ID NO: 76 - DNA, 23 bp, Artificial
  downstream primer for detecting ERBB3 expression amount
ctgagacccactgaagaaagggt

SEQ ID NO: 77 - DNA, 20 bp, Artificial
  upstream primer for detecting TOP2A expression amount
cagcgtgttgagcctgaatg

SEQ ID NO: 78 - DNA, 22 bp, Artificial
  upstream primer for detecting TOP2A expression amount
gcgtgttgagcctgaatggtac

SEQ ID NO: 79 - DNA, 20 bp, Artificial
  downstream primer for detecting TOP2A expression amount
aggaccacccagtaccgatt

SEQ ID NO: 80 - DNA, 22 bp, Artificial
  downstream primer for detecting TOP2A expression amount
ggaccacccagtaccgattcct

SEQ ID NO: 81 - DNA, 19 bp, Artificial
  upstream primer for detecting TYMS expression amount
ggcaccctgtcggtattcg

SEQ ID NO: 82 - DNA, 18 bp, Artificial
  upstream primer for detecting TYMS expression amount
ggcaccctgtcggtattc

SEQ ID NO: 83 - DNA, 19 bp, Artificial
  downstream primer for detecting TYMS expression amount
cccttccagaacacacgtt

SEQ ID NO: 84 - DNA, 21 bp, Artificial
  downstream primer for detecting TYMS expression amount
ctccaaaacacccttccagaa

SEQ ID NO: 85 - DNA, 22 bp, Artificial
  upstream primer for detecting RAP-80 expression amount
caaaatgagtgagcaggaagct

SEQ ID NO: 86 - DNA, 19 bp, Artificial
  upstream primer for detecting RAP-80 expression amount
aatgagtgagcaggaagct

SEQ ID NO: 87 - DNA, 20 bp, Artificial
  downstream primer for detecting RAP-80 expression amount
tcagaaggccggcaactatt

SEQ ID NO: 88 - DNA, 23 bp, Artificial
  downstream primer for detecting RAP-80 expression amount
caactattcaggctttcagcaat

SEQ ID NO: 89 - DNA, 21 bp, Artificial
  upstream primer for detecting VEGFR1 expression amount
tttaccgaatgccacctccat

SEQ ID NO: 90 - DNA, 17 bp, Artificial
  upstream primer for detecting VEGFR1 expression amount
ccgaatgccacctccat

SEQ ID NO: 91 - DNA, 20 bp, Artificial
  downstream primer for detecting VEGFR1 expression amount
atgggagaggccaacagagt

SEQ ID NO: 92 - DNA, 18 bp, Artificial
  downstream primer for detecting VEGFR1 expression amount
gggagaggccaacagagt

SEQ ID NO: 93 - DNA, 21 bp, Artificial
  upstream primer for detecting VEGFR2 expression amount
tggagcaatccctgtggatct

SEQ ID NO: 94 - DNA, 20 bp, Artificial
  upstream primer for detecting VEGFR2 expression amount
ggagcaatccctgtggatct

SEQ ID NO: 95 - DNA, 21 bp, Artificial
  downstream primer for detecting VEGFR2 expression amount
ctcctccacaaatccagagct

SEQ ID NO: 96 - DNA, 19 bp, Artificial
  downstream primer for detecting VEGFR2 expression amount
cctccacaaatccagagct

SEQ ID NO: 97 - DNA, 24 bp, Artificial
  upstream primer for detecting HER2 expression amount
gaaggacatcttccacaagaacaa

SEQ ID NO: 98 - DNA, 20 bp, Artificial
  upstream primer for detecting HER2 expression amount
tgctgtcctgttcaccactc

SEQ ID NO: 99 - DNA, 22 bp, Artificial
  downstream primer for detecting HER2 expression amount
gagcccttacacatcggagaac

SEQ ID NO: 100 - DNA, 20 bp, Artificial
  downstream primer for detecting HER2 expression amount
gctttgcatgtggtcttgaa

SEQ ID NO: 101 - DNA, 20 bp, Artificial
  upstream primer for detecting EGFR expression amount
ccctcctgagctctctgagt

SEQ ID NO: 102 - DNA, 16 bp, Artificial
  upstream primer for detecting EGFR expression amount
tgcaaccagcaacaat

SEQ ID NO: 103 - DNA, 20 bp, Artificial
  downstream primer for detecting EGFR expression amount
cttgatgggacagctttgca

SEQ ID NO: 104 - DNA, 18 bp, Artificial
  upstream primer for detecting EGFR expression amount
gaagctgtcttccttgat

SEQ ID NO: 105 - DNA, 19 bp, Artificial
  upstream primer for detecting VEGF expression amount
gccttgccttgctgctcta

SEQ ID NO: 106 - DNA, 20 bp, Artificial
  upstream primer for detecting VEGF expression amount
ctgctgtcttgggtgcattg

SEQ ID NO: 107 - DNA, 21 bp, Artificial
  downstream primer for detecting VEGF expression amount
tgattctgccctcctccttct

SEQ ID NO: 108 - DNA, 20 bp, Artificial
  downstream primer for detecting VEGF expression amount
gattctgccctcctccttct

SEQ ID NO: 109 - DNA, 20 bp, Artificial
  upstream primer for detecting PPN expression amount
tcctcagtgcctgtgtcttg

SEQ ID NO: 110 - DNA, 17 bp, Artificial
  upstream primer for detecting PPN expression amount
gatgtcgatgtggatga

SEQ ID NO: 111 - DNA, 20 bp, Artificial
  downstream primer for detecting PPN expression amount
gcatccaaaagtgacccagt

SEQ ID NO: 112 - DNA, 19 bp, Artificial
  downstream primer for detecting PPN expression amount
cacttgtggacagtgtatg

SEQ ID NO: 113 - DNA, 21 bp, Artificial
  upstream primer for detecting CCNB2 expression amount
ggacattgataacgaagattg

SEQ ID NO: 114 - DNA, 20 bp, Artificial
  upstream primer for detecting CCNB2 expression amount
cacaggatacacagagaatg

SEQ ID NO: 115 - DNA, 18 bp, Artificial
  downstream primer for detecting CCNB2 expression amount
gctgcctgagatactgat

SEQ ID NO: 116 - DNA, 20 bp, Artificial
  downstream primer for detecting CCNB2 expression amount
cttgatggcgatgaatttag

SEQ ID NO: 117 - DNA, 20 bp, Artificial
  upstream primer for detecting ACTB expression amount
cagatgtggatcagcaagca

SEQ ID NO: 118 - DNA, 20 bp, Artificial
  upstream primer for detecting ACTB expression amount
agaaaatctggcaccacacc

SEQ ID NO: 119 - DNA, 22 bp, Artificial
  downstream primer for detecting ACTB expression amount
tcatagtccgcctagaagcatt

SEQ ID NO: 120 - DNA, 20 bp, Artificial
  downstream primer for detecting ACTB expression amount
agaggcgtacagggatagca

SEQ ID NO: 121 - DNA, 20 bp, Artificial
  upstream primer for detecting 18S rRNA expression amount
acatccaaggaaggcagcag

SEQ ID NO: 122 - DNA, 20 bp, Artificial
  upstream primer for detecting 18S rRNA expression amount
acatccaaggaaggcagcag

SEQ ID NO: 123 - DNA, 19 bp, Artificial
  downstream primer for detecting 18S rRNA expression amount
ttcgtcactacctccccgg

SEQ ID NO: 124 - DNA, 19 bp, Artificial
  downstream primer for detecting 18S rRNA expression amount
ttcgtcactacctccccgg

SEQ ID NO: 125 - DNA, 22 bp, Artificial
  upstream primer for detecting HER2 gene amplification
cagaccatttgggttcaaatcc

SEQ ID NO: 126 - DNA, 23 bp, Artificial
  upstream primer for detecting HER2 gene amplification
aaagttcctttagctcgtggaat

SEQ ID NO: 127 - DNA, 21 bp, Artificial
  downstream primer for detecting HER2 gene amplification
gagaccaaagcagagagttct

SEQ ID NO: 128 - DNA, 21 bp, Artificial
  downstream primer for detecting HER2 gene amplification
aggaggcaggtgttatcattc

SEQ ID NO: 129 - DNA, 22 bp, Artificial
  upstream primer for ACTB
caggatgcagaaggagatcact

SEQ ID NO: 130 - DNA, 18 bp, Artificial
  upstream primer for ACTB
catcctcaccctgaagta

SEQ ID NO: 131 - DNA, 20 bp, Artificial
  downstream primer for ACTB
cagctcaggcaggaaagaca

SEQ ID NO: 132 - DNA, 18 bp, Artificial
  downstream primer for ACTB
acacgcagctcattgtag

SEQ ID NO: 133 - DNA, 18 bp, Artificial
  wild-type probe for EGFR Exon 18
ggctccggtgcgttcggc

SEQ ID NO: 134 - DNA, 21 bp, Artificial
  wild-type probe for EGFR Exon 18
cggagcccagcactttgatct

SEQ ID NO: 135 - DNA, 19 bp, Artificial
  probe for EGFR Exon 18 position 2155 G->A mutant
tgctgagctccggtgcgtt

SEQ ID NO: 136 - DNA, 22 bp, Artificial
  probe for EGFR Exon 18 position 2155 G->A mutant
cggagctcagcactttgatctt

SEQ ID NO: 137 - DNA, 25 bp, Artificial
  wild-type probe for EGFR Exon 19
tcaaggaattaagagaagcaacatc

SEQ ID NO: 138 - DNA, 26 bp, Artificial
  wild-type probe for EGFR Exon 19
cggagatgttgcttctcttaattcct

SEQ ID NO: 139 - DNA, 21 bp, Artificial
  probe for EGFR Exon 19 position 2235-2249 deletion
cccgtcgctatcaaaacatct

SEQ ID NO: 140 - DNA, 21 bp, Artificial
  probe for EGFR Exon 19 position 2235-2249 deletion
agatgttttgatagcgacggg

SEQ ID NO: 141 - DNA, 22 bp, Artificial
  probe for EGFR Exon 19 position 2236-2250 deletion
cccgtcgctatcaagacatctc

SEQ ID NO: 142 - DNA, 22 bp, Artificial
  probe for EGFR Exon 19 position 2236-2250 deletion
gagatgtcttgatagcgacggg

SEQ ID NO: 143 - DNA, 23 bp, Artificial
  probe for EGFR Exon 19 position 2254-2277 deletion
aattaagagaagcaacactcgat

SEQ ID NO: 144 - DNA, 23 bp, Artificial
  probe for EGFR Exon 19 position 2254-2277 deletion
atcgagtgttgcttctcttaatt

SEQ ID NO: 145 - DNA, 20 bp, Artificial
  wild-type probe for EGFR Exon 21
tggccagcccaaaatctgtg

SEQ ID NO: 146 - DNA, 23 bp, Artificial
  wild-type probe for EGFR Exon 21
aagatcacagattttgggctggc

SEQ ID NO: 147 - DNA, 19 bp, Artificial
  probe for EGFR Exon 21 position 2573 T->G mutant
tggcccgcccaaaatctgt

SEQ ID NO: 148 - DNA, 21 bp, Artificial
  probe for EGFR Exon 21 position 2573 T->G mutant
gatcacagattttgggcgggc

SEQ ID NO: 149 - DNA, 20 bp, Artificial
  wild-type probe for K-ras
agctggtggcgtaggcaaga

SEQ ID NO: 150 - DNA, 22 bp, Artificial
  wild-type probe for K-ras
agctggtggcgtaggcaagagt

SEQ ID NO: 151 - DNA, 20 bp, Artificial
  probe for K-ras Codon 12 GGT->GTT mutant
agctgttggcgtaggcaaga

SEQ ID NO: 152 - DNA, 23 bp, Artificial
  probe for K-ras Codon 12 GGT->GTT mutant
agctgttggcgtaggcaagagtg

SEQ ID NO: 153 - DNA, 20 bp, Artificial
  probe for K-ras Codon 12 GGT->AGT mutant
agctagtggcgtaggcaaga

SEQ ID NO: 154 - DNA, 23 bp, Artificial
  probe for K-ras Codon 12 GGT->AGT mutant
agctagtggcgtaggcaagagtg

SEQ ID NO: 155 - DNA, 20 bp, Artificial
  probe for K-ras Codon 12 GGT->GAT mutant
agctgatggcgtaggcaaga

SEQ ID NO: 156 - DNA, 23 bp, Artificial
  probe for K-ras Codon 12 GGT->GAT mutant
agctgatggcgtaggcaagagtg

SEQ ID NO: 157 - DNA, 20 bp, Artificial
  probe for K-ras Codon 12 GGT->TGT mutant
agcttgtggcgtaggcaaga

SEQ ID NO: 158 - DNA, 23 bp, Artificial
  probe for K-ras Codon 12 GGT->TGT mutant
agcttgtggcgtaggcaagagtg

SEQ ID NO: 159 - DNA, 20 bp, Artificial
  probe for K-ras Codon 13 GGC->GAC mutant
agctggtgacgtaggcaaga

SEQ ID NO: 160 - DNA, 23 bp, Artificial
  probe for K-ras Codon 13 GGC->GAC mutant
agctggtgacgtaggcaagagtg

SEQ ID NO: 161 - DNA, 24 bp, Artificial
  wild-type probe for BCRP Codon 482
cccatgaggatgttaccaagtatt

SEQ ID NO: 162 - DNA, 27 bp, Artificial
  wild-type probe for BCRP  Codon 482
ttacccatgaggatgttaccaagtatt

SEQ ID NO: 163 - DNA, 24 bp, Artificial
  probe for BCRP Codon 482 AGG->GGG mutant
cccatggggatgttaccaagtatt

SEQ ID NO: 164 - DNA, 27 bp, Artificial
  probe for BCRP Codon 482 AGG->GGG mutant
ttacccatggggatgttaccaagtatt

SEQ ID NO: 165 - DNA, 24 bp, Artificial
  probe for BCRP Codon 482 AGG->ACG mutant
cccatgacgatgttaccaagtatt

SEQ ID NO: 166 - DNA, 27 bp, Artificial
  probe for BCRP Codon 482 AGG->ACG mutant
ttacccatgacgatgttaccaagtatt

SEQ ID NO: 167 - DNA, 20 bp, Artificial
  wild-type probe for BRAF Codon 600
ccatcgagatttcactgtag

SEQ ID NO: 168 - DNA, 28 bp, Artificial
  wild-type probe for BRAF Codon 600
ccatcgagatttcactgtagctagacca

SEQ ID NO: 169 - DNA, 20 bp, Artificial
  probe for BRAF Codon 600 GTG->GAG mutant
ccatcgagatttctctgtag

SEQ ID NO: 170 - DNA, 28 bp, Artificial
  probe for BRAF Codon 600 GTG->GAG mutant
ccatcgagatttctctgtagctagacca

SEQ ID NO: 171 - DNA, 23 bp, Artificial
  probe for detecting ERCC1 expression amount
cccgactatgtgctgggccagag

SEQ ID NO: 172 - DNA, 25 bp, Artificial
  probe for detecting ERCC1 expression amount
cacaggtgctctggcccagcacata

SEQ ID NO: 173 - DNA, 27 bp, Artificial
  probe for detecting RRM1 expression amount
caggatcgctgtctctaacttgcacaa

SEQ ID NO: 174 - DNA, 28 bp, Artificial
  probe for detecting RRM1 expression amount
cagccaggatcgctgtctctaacttgca

SEQ ID NO: 175 - DNA, 27 bp, Artificial
  probe for detecting BRCA1 expression amount
ccgtgccaaaagacttctacagagtga

SEQ ID NO: 176 - DNA, 23 bp, Artificial
  probe for detecting BRCA1 expression amount
gccaaaagacttctacagagtga

SEQ ID NO: 177 - DNA, 25 bp, Artificial
  probe for detecting TUBB3 expression amount
cacaggtggcaaatatgttcctcgt

SEQ ID NO: 178 - DNA, 20 bp, Artificial
  probe for detecting TUBB3 expression amount
caggtggcaaatatgttcct

SEQ ID NO: 179 - DNA, 23 bp, Artificial
  probe for detecting ERBB3 expression amount
aaaggtactccctcctcccggga

SEQ ID NO: 180 - DNA, 20 bp, Artificial
  probe for detecting ERBB3 expression amount
ggtactccctcctcccggga

SEQ ID NO: 181 - DNA, 27 bp, Artificial
  probe for detecting TOP2A expression amount
cttcagcaccatttatcagcaccatgg

SEQ ID NO: 182 - DNA, 20 bp, Artificial
  probe for detecting TOP2A expression amount
ttcagcaccatttatcagca

SEQ ID NO: 183 - DNA, 23 bp, Artificial
  probe for detecting TYMS expression amount
cgcgctacagcctgagagatgaa

SEQ ID NO: 184 - DNA, 19 bp, Artificial
  probe for detecting TYMS expression amount
cgctacagcctgagagatg

SEQ ID NO: 185 - DNA, 24 bp, Artificial
  probe for detecting RAP-80 expression amount
cagccaggaggaggaagaagagga

SEQ ID NO: 186 - DNA, 19 bp, Artificial
  probe for detecting RAP-80 expression amount
gccaggaggaggaagaaga

SEQ ID NO: 187 - DNA, 25 bp, Artificial
  probe for detecting VEGFR1 expression amount
ctgctgtcgccctggtagtcatcaa

SEQ ID NO: 188 - DNA, 19 bp, Artificial
  probe for detecting VEGFR1 expression amount
ctgtcgccctggtagtcat

SEQ ID NO: 189 - DNA, 25 bp, Artificial
  probe for detecting VEGFR2 expression amount
acggcgcttggacagcatcaccagt

SEQ ID NO: 190 - DNA, 21 bp, Artificial
  probe for detecting VEGFR2 expression amount
cggcgcttggacagcatcacc

SEQ ID NO: 191 - DNA, 26 bp, Artificial
  probe for detecting HER2 expression amount
ctctcacactgatagacaccaaccgc

SEQ ID NO: 192 - DNA, 24 bp, Artificial
  probe for detecting HER2 expression amount
cggtgtgagaagtgcagcaagccc

SEQ ID NO: 193 - DNA, 24 bp, Artificial
  probe for detecting EGFR expression amount
caattccaccgtggcttgcattga

SEQ ID NO: 194 - DNA, 22 bp, Artificial
  probe for detecting EGFR expression amount
tccaccgtggcttgcattgata

SEQ ID NO: 195 - DNA, 23 bp, Artificial
  probe for detecting VEGF expression amount
ctccaccatgccaagtggtccca

SEQ ID NO: 196 - DNA, 20 bp, Artificial
  probe for detecting VEGF expression amount
ccaccatgccaagtggtccc

SEQ ID NO: 197 - DNA, 22 bp, Artificial
  probe for detecting PPN expression amount
caccacagaggagcagggctac

SEQ ID NO: 198 - DNA, 21 bp, Artificial
  probe for detecting PPN expression amount
agcatgtcctccggaagcgcc

SEQ ID NO: 199 - DNA, 23 bp, Artificial
  probe for detecting CCNB2 expression amount
agaaccctcagctctgcagtgac

SEQ ID NO: 200 - DNA, 24 bp, Artificial
  probe for detecting CCNB2 expression amount
attggaagtcatgcagcacatggc

SEQ ID NO: 201 - DNA, 20 bp, Artificial
  probe for detecting ACTB expression amount
cccatcgagcacggcatcgt

SEQ ID NO: 202 - DNA, 23 bp, Artificial
  probe for detecting ACTB expression amount
atgacgagtccggcccctccatc

SEQ ID NO: 203 - DNA, 19 bp, Artificial
  probe for detecting 18S rRNA expression amount
tggtgtcgcggagcacgga

SEQ ID NO: 204 - DNA, 22 bp, Artificial
  probe for detecting 18S rRNA expression amount
cgcgcaaattacccactcccga

SEQ ID NO: 205 - DNA, 25 bp, Artificial
  probe for detecting HER2 gene amplification
ctcctccactcactagcacaatgac

SEQ ID NO: 206 - DNA, 25 bp, Artificial
  probe for detecting HER2 gene amplification
tcaaggctcaaggttcctcttctgc

SEQ ID NO: 207 - DNA, 24 bp, Artificial
  probe for detecting ACTB gene amplification
tggcacccagcacaatgaagatca

SEQ ID NO: 208 - DNA, 20 bp, Artificial
  probe for detecting ACTB gene amplification
cccatcgagcacggcatcgt

SEQ ID NO: 209 - DNA, 18 bp, unknown
  DNA sequence containing base G at EGFR Exon 18 position 2155
aaagtgctgggctccggt

SEQ ID NO: 210 - DNA, 18 bp, unknown
  DNA sequence containing base A at EGFR Exon 18 position 2155
aaagtgctgggctccggt

SEQ ID NO: 211 - DNA, 53 bp, unknown
  DNA sequence containing wild-type sequence at EGFR Exon 19 position 2235-2277
tcaaggaattaagagaagcaacatctccgaaagccaacaaggaaatcctcgat

SEQ ID NO: 212 - DNA, 38 bp, unknown
  DNA sequence containing deletion mutant at EGFR Exon 19 position 2235-2249
cgctatcaaaacatctccgaaagccaacaaggaaatcc

SEQ ID NO: 213 - DNA, 39 bp, unknown
  DNA sequence containing deletion mutant at EGFR Exon 19 position 2236-2250
cgtcgctatcaagacatctccgaaagccaacaaggaaat

SEQ ID NO: 214 - DNA, 35 bp, unknown
  DNA sequence containing deletion mutant at EGFR Exon 19 position 2254-2277
aagagaagcaacactcgatgtgagtttctgctttg

SEQ ID NO: 215 - DNA, 18 bp, unknown
  DNA sequence containing base T at EGFR Exon 21 position 2573
acagattttgggctggcc

SEQ ID NO: 216 - DNA, 18 bp, unknown
  DNA sequence containing base G at EGFR Exon 21 position 2573
acagattttgggcgggcc

SEQ ID NO: 217 - DNA, 41 bp, unknown
  DNA sequence containing K-ras Codon 12, 13 wild-type sequence
cttgtggtagttggagctggtggcgtaggcaagagtgcctt

SEQ ID NO: 218 - DNA, 41 bp, unknown
  DNA sequence containing GTT mutant at K-ras Codon 12
cttgtggtagttggagctgttggcgtaggcaagagtgcctt

SEQ ID NO: 219 - DNA, 40 bp, unknown
  DNA sequence containing AGT mutant at K-ras Codon 12
ttgtggtagttggagctagtggcgtaggcaagagtgcctt

SEQ ID NO: 220 - DNA, 40 bp, unknown
  DNA sequence containing GAT mutant at K-ras Codon 12
ttgtggtagttggagctgatggcgtaggcaagagtgcctt

SEQ ID NO: 221 - DNA, 41 bp, unknown
  DNA sequence containing TGT mutant at K-ras Codon 12
cttgtggtagttggagcttgtggcgtaggcaagagtgcctt

SEQ ID NO: 222 - DNA, 40 bp, unknown
  DNA sequence containing GAC mutant at K-ras Codon 13
ttgtggtagttggagctggtgacgtaggcaagagtgcctt

SEQ ID NO: 223 - DNA, 45 bp, unknown
  DNA sequence containing wild-type sequence at BCRP Codon 482
actgttatctgatttattacccatgaggatgttaccaagtattat

SEQ ID NO: 224 - DNA, 45 bp, unknown
  DNA sequence containing GGG mutant at BCRP Codon 482
actgttatctgatttattacccatggggatgttaccaagtattat

SEQ ID NO: 225 - DNA, 45 bp, unknown
  DNA sequence containing ACG mutant at BCRP Codon 482
actgttatctgatttattacccatgacgatgttaccaagtattat

SEQ ID NO: 226 - DNA, 26 bp, unknown
  DNA sequence containing wild-type sequence at BRAF Codon 600
ggtctagctacagtgaaatctcgatg

SEQ ID NO: 227 - DNA, 26 bp, unknown
  DNA sequence containing GAG mutant at BRAF Codon 600
ggtctagctacagagaaatctcgatg

SEQ ID NO: 228 - DNA, 71 bp, unknown
  DNA sequence containing ERCC1 cDNA sequence
gcggaggctgaggaacagggcacaggtgctctggcccagcacatagtcgggaattacgtcgccaaattccc

SEQ ID NO: 229 - DNA, 99 bp, unknown
  DNA sequence containing RRM1 cDNA sequence
cttccatcacatcactgaacactttctttgtttctttgtgcaagttagagacagcgatcctggctgccaggatagcatagtcagggtgcttagtagtca

SEQ ID NO: 230 - DNA, 61 bp, unknown
  DNA sequence containing BRCA1 cDNA sequence
tcatccaaagtatgggctacagaaaccgtgccaaaagacttctacagagtgaacccgaaaa

SEQ ID NO: 231 - DNA, 90 bp, unknown
  DNA sequence containing TUBB3 cDNA sequence
gaccgcatctctgtgtactacaatgaagccacaggtggcaaatatgttcctcgtgccatcctggtggatctagaacctgggaccatggac

SEQ ID NO: 232 - DNA, 87 bp, unknown
  DNA sequence containing ERBB3 cDNA sequence
gacagaactgagacccactgaagaaagggtgccttcccgggaggagggagtacctttgaggtgtgtatctggcatgacataaccgtt

SEQ ID NO: 233 - DNA, 85 bp, unknown
  DNA sequence containing TOP2A cDNA sequence
aggaccacccagtaccgattccttcagcaccatttatcagcaccatgggaataataggaatgtaccattcaggctcaacacgctg

SEQ ID NO: 234 - DNA, 89 bp, unknown
  DNA sequence containing TYMS cDNA sequence
cccttccagaacacacgttggtgcccgtgcggtcgtccttcctgacgccgcagcggaggatgtgttggaaatcgtcgacctgcaggcat

SEQ ID NO: 235 - DNA, 108 bp, unknown
  DNA sequence containing RAP-80 cDNA sequence
caaaatgagtgagcaggaagctagggaggtgaacagccaggaggaggaagaagaggagctcttgaggaaagccattgctgaaagcctgaatagttgccggccttctga

SEQ ID NO: 236 - DNA, 69 bp, unknown
  DNA sequence containing VEGFR1 cDNA sequence
tttaccgaatgccacctccatgtttgatgactaccagggcgacagcagcactctgttggcctctcccat

SEQ ID NO: 237 - DNA, 85 bp, unknown
  DNA sequence containing VEGFR2 cDNA sequence
ctcctccacaaatccagagctggctgagctctggctactggtgatgctgtccaagcgccgtttcagatccacagggattgctcca

SEQ ID NO: 238 - DNA, 99 bp, unknown
  DNA sequence containing HER2 cDNA sequence
gagcccttacacatcggagaacaggggtggcaggcccgagagcggttggtgtctatcagtgtgagagccagctggttgttcttgtggaagatgtccttc

SEQ ID NO: 239 - DNA, 86 bp, unknown
  DNA sequence containing EGFR cDNA sequence
cttgatgggacagctttgcagagtccgtgacgtggaggggctgctgaagaagccctgctgtgggatgaggtactcgtcggcatcca

SEQ ID NO: 240 - DNA, 79 bp, unknown
  DNA sequence containing VEGF cDNA sequence
gccttgccttgctgctctacctccaccatgccaagtggtcccaggctgcacccatggcagaaggaggagggcagaatca

SEQ ID NO: 241 - DNA, 248 bp, unknown
  DNA sequence containing PPN cDNA sequence
gcatccaaaagtgacccagtgactggcccagctgagctctgaccacttgtggacagtgtatgccatgccgtagccctgctcctctgtggtgtcatccacatcgacatcaaacagggagcccaggtaggccaggtggaagatggccagagctccaaagagcaagtttaaggctcgcacccccaggcccaagcgatgctggtgcgaacagtctggcgggcaccgctttgacaagacacaggcactgagga

SEQ ID NO: 242 - DNA, 80 bp, unknown
  DNA sequence containing CCNB2 cDNA sequence
gctgcctgagatactgatagatatccttaacgtagtcactgcagagctgagggttctcccaatcttcgttatcaatgtcc

SEQ ID NO: 243 - DNA, 80 bp, unknown
  DNA sequence containing ACTB cDNA sequence
cagatgtggatcagcaagcaggagtatgacgagtccggcccctccatcgtccaccgcaaatgcttctaggcggactatga

SEQ ID NO: 244 - DNA, 63 bp, unknown
  DNA sequence containing 18S rRNA cDNA sequence
acatccaaggaaggcagcaggcgcgcaaattacccactcccgacccggggaggtagtgacgaa

SEQ ID NO: 245 - DNA, 470 bp, unknown
  DNA sequence containing HER2 genome DNA
gcctgcaggtcgacgattggaagtacacgatgcggagactgctgcaggaaacggaggtgaggcggggtgaagtcctcccagcccgcgtggggtctgcaccggcccccggcactgacccaccaccccctcaccccagctggtggagccgctgacacctagcggagcgatgcccaaccaggcgcagatgcggatcctgaaagagacggagctgaggaaggtgaaggtgcttggatctggcgcttttggcacagtctacaaggtcagggccaggtcctggggtgggcggccccagaggatgggggcggtgcctggaggggtgtggtcggcagttctgatgggaggggcaagagctggaggcagtgtttgggggagggcagttacagcggagaagggagcggggccaagccctagggtggtgaaggatgtttggaggacaagtaatgatctcctggaaggcaggtaggatccag

SEQ ID NO: 246 - DNA, 77 bp, unknown
  DNA sequence containing ACTB genome DNA
caggatgcagaaggagatcactgccctggcacccagcacaatgaagatcaaggtgggtgtctttcctgcctgagctg

(19) United States
(12) Patent Application Publication
(10) Pub. No.: US 2013/0217115 A1
(43) Pub. Date: Aug. 22, 2013

(54) PLASMID STANDARD FOR USE IN QUANTITATIVE ASSAYS USING FLUORESCENT QUANTITATIVE PCR

(71) Applicants: Junpu Xu, Beijing (CN); Zhao Chen, Beijing (CN); Minli Mo, Beijing (CN); Jun Li, Beijing (CN)

(72) Inventors: Junpu Xu, Beijing (CN); Zhao Chen, Beijing (CN); Minli Mo, Beijing (CN); Jun Li, Beijing (CN)

(21) Appl. No.: 13/880,373

(22) PCT Filed: Oct. 18, 2011
(86) PCT No.: PCT/CN2011/001738

§ 371 Date: Apr. 18, 2013

(30) Foreign Application Priority Data
Oct. 18, 2010 (CN) 201010509523.0

(51) Int. Cl. C12Q 1/68

(52) U.S. Cl. CPC C12Q 1/6876
USPC 435/320.1

(57) ABSTRACT

Disclosed is a plasmid standard for use in fluorescent quantitative PCR assays. More specifically, the present invention provides a plasmid standard as well as amplification primers and detection probes thereof for use in the detection of gene mutation and expression amount.

### CROSS REFERENCE TO RELATED APPLICATIONS

This application claims the priority of Chinese Patent Application No. 201010509523.0, filed on Oct. 18, 2010, the disclosure of which is incorporated herein by reference.

### TECHNICAL FIELD

The present invention relates to plasmid. Specifically, the present invention relates to plasmid standards for quantitative detection by fluorescent quantitative PCR.

### BACKGROUND OF THE INVENTION

Fluorescent quantitative PCR was first reported by Higuchi, a Japanese scientist, in 1992. It refers to adding a fluorescent group into PCR reaction system. The variation of the fluorescent energy emitted under light stimulation directly reflects the variation of PCR amplification product. The variation of fluorescent signal is in proportion to that of amplification product. It is possible to quantify the amount of original template by collecting and analyzing the fluorescent signal using an automated instrument with sufficient sensitivity, and finally analyzing the unknown template via a standard curve.

There are two methods to quantify the template in fluorescent real-time PCR: absolute quantification method and relative quantification method [Walker N J, J Biochem Mol Toxicol, 2001, 15(3): 121-127]. Absolute quantification method is a method for determining the absolute amount (copy number) of an unknown sample. Contrarily, the relative quantification method is not a method for determining the absolute amount of a gene. It is for determining the amounts of a gene of interest and internal reference gene respectively, normalizing the amount of gene of interest to that of the internal reference gene, and then comparing the relative amount among samples.

The analysis method for absolute quantification to preparing a standard curve using a standard with known concentration, and then testing the absolute amount (copy number) of the sample with unknown concentration. Therefore, the standard which has a known absolute amount (copy number) and contains unknown sample sequence is necessary. To keep consistency with the PCR amplification efficiency of the sample to be tested, the standard should be chosen so that its structure is similar to that of the sample to be tested. It has been verified that plasmid standard molecule is a very good substitute for the standard positive substance in GMO identification test. The advantages of plasmid are: they can be obtained in large scale via microorganism cultivation, the DNA can be easily amplified, so that it is possible to provide unlimited amount of standard substance with high purity. The operation is easy and stable. Moreover, one standard molecule can contain several exogenous target genes, which means economical and high efficacy. Thus, the plasmid standard molecule can be called “golden standard substance”.

To prepare a standard curve, one should prepare 4-6 gradiently diluted standards, then obtain individual Ct value for each one of them by Real Time PCR using these standards as templates. The standard curve can be prepared based on the linear relationship between the Ct values and the logarithm values of original template concentrations.

Although molecular mechanisms of tumorigenesis are not fully elucidated, it is commonly accepted that accumulation of genetic changes of related genes is the fundamental cause for the change of carcinogenicity. The increase of expression and mutation of oncogene can occur at early and benign stage in many tumors. Fluorescent quantitative real-time PCR can not only efficiently detect gene mutations, but also accurately determine amount of gene expressions. Thus, it is possible to carry out early diagnosis, treatment and prognosis of tumor based on this technology. The detection of genetic changes for some oncogenes can make almost definite diagnosis.

The plasmid vector for fluorescent quantitative real-time PCR used in the present invention has the following advantages:

1. The process of preparation and treatment is simple and easy, the experimental cycle is short, and the experimental procedures can be easily standardized.

2. The price is moderate, and thus it can be easily adopted.

3. The unique advantage of the present invention is accurate quantification. Using fluorescent real-time PCR amplification curve parameters, we can quantatively determine the copy number of genes in the samples.

4. Human errors during experiments can be reduced.

### SUMMARY OF THE INVENTION

The question that the present invention addresses is to provide a positive standards for gene mutation detection, expression detection and gene amplification detection.

To address the above question, the present invention provides the following solutions:

(1) constructing a plasmid vector, which contains a gene sequence to be detected.

(2) a plasmid vector according to (1) above, which is selected from TA clone vector, preferably pMD18-T.

(3) a plasmid vector according to (1) above, wherein the gene to be detected is integrated into the vector.

### BRIEF DESCRIPTION OF DRAWINGS

The accompanying drawings, which are incorporated in and form a part of this specification, illustrate embodiments of the technology and together with the description.

FIG. 1 is a diagram showing the method for constructing the plasmid control in Example 2.

FIG. 2 is a diagram showing the plasmid profile of Example 2, wherein the PCR product sequence is inserted into the vector at the position marked with an arrow.

FIG. 3 is a diagram showing the result of sequencing the wild-type plasmid standard of Example 2, wherein Fig.A is the sequencing result of EGFR Exon 18 position 2155G wild-type plasmid, Fig.B is the sequencing result of EGFR Exon 19 position 2235-2249, position 2236-2250, and position 2254-2277 wild-type plasmid, Fig.C is the sequencing result of EGFR Exon 21 position 2573T wild-type plasmid

FIG. 4 is a diagram showing the result of sequencing the mutant plasmid standard of Example 2, wherein the mutant site is marked with an arrow. Fig.A is the sequencing result of EGFR Exon 18 position 2155 G→A mutant plasmid, Fig.B is the sequencing result of EGFR Exon 19 position 2235-2249 deletion plasmid, FIG.C is the sequencing result of EGFR Exon 19 position 2236-2250 deletion plasmid, Fig.D is the sequencing result of EGFR Exon 19 position 2254-2277 deletion plasmid, Fig.E is the sequencing result of EGFR Exon 21 position 2573 T→G mutant plasmid.

FIG. 5 is a diagram showing the result of sequencing the KRAS wild-type plasmid of Example 3.

FIG. 6 is a diagram showing the result of sequencing the mutant plasmid standard of Example 3, wherein the mutant site is marked with an arrow. Fig.A is the sequencing result of KRAS Codon 12 GGT→GTT mutant plasmid, Fig.B is the sequencing result of KRAS Codon 12 GGT→AGT mutant plasmid, FIG.C is the sequencing result of KRAS Codon 12 GGT→GAT mutant plasmid, Fig.D is the sequencing result of KRAS codon 12 GGT→TGT mutant plasmid, Fig.E is the sequencing result of KRAS codon 13 GGC→GAC mutant plasmid.

FIG. 7 is a diagram showing the result of sequencing the BCRP wild-type plasmid of Example 4.

FIG. 8 is a diagram showing the result of sequencing the mutant plasmid standard of Example 4, wherein the mutant site is marked with an arrow. Fig.A is the sequencing result of BCRP Codon 482 AGG→GGG mutant plasmid, Fig.B is the sequencing result of BCRP Codon 482 AGG→ACG mutant plasmid.

FIG. 9 is a diagram showing the result of sequencing the BRAF wild-type plasmid of Example 5.

FIG. 10 is a diagram showing the result of sequencing the mutant plasmid standard of Example 5, wherein the mutant site is marked with an arrow.

FIG. 11 is a diagram showing the result of sequencing the plasmid for detecting ERCC1 expression in Example 6.

FIG. 12 is a diagram showing the result of sequencing the plasmid for detecting RRM1 expression in Example 7.

FIG. 13 is a diagram showing the result of sequencing the plasmid for detecting BRCA1 expression in Example 8.

FIG. 14 is a diagram showing the result of sequencing the plasmid for detecting TUBB3 expression in Example 9.

FIG. 15 is a diagram showing the result of sequencing the plasmid for detecting ERBB3 expression in Example 10.

FIG. 16 is a diagram showing the result of sequencing the plasmid for detecting TOP2A expression in Example 11.

FIG. 17 is a diagram showing the result of sequencing the plasmid for detecting TYMS expression in Example 12.

FIG. 18 is a diagram showing the result of sequencing the plasmid for detecting RAP-80 expression in Example 13.

FIG. 19 is a diagram showing the result of sequencing the plasmid for detecting VEGFR1 expression in Example 14.

FIG. 20 is a diagram showing the result of sequencing the plasmid for detecting VEGFR2 expression in Example 15.

FIG. 21 is a diagram showing the result of sequencing the plasmid for detecting HER2 expression in Example 16.

FIG. 22 is a diagram showing the result of sequencing the plasmid for detecting EGFR expression in Example 17.

FIG. 23 is a diagram showing the result of sequencing the plasmid for detecting VEGF expression in Example 18.

FIG. 24 is a diagram showing the result of sequencing the plasmid for detecting PPN expression in Example 19.

FIG. 25 is a diagram showing the result of sequencing the plasmid for detecting CCNB2 expression in Example 20.

FIG. 26 is a diagram showing the result of sequencing the plasmid for detecting ACTB expression in Example 21.

FIG. 27 is a diagram showing the result of sequencing the plasmid for detecting 18S rRNA expression in Example 22.

FIG. 28 is a diagram showing the result of sequencing the plasmid for detecting HER2 gene amplification in Example 23.

FIG. 29 is a diagram showing the result of sequencing the plasmid for detecting ACTB gene amplification in Example 8.

FIG. 30 is a diagram showing the amplification curve of the plasmid standard of Example 25. Fig.A1 shows the amplification curve of EGFR Exon 18 position 2155G wild-type plasmid standard, Fig.A2 shows the amplification curve of EGFR Exon 19 position 2235-2249, 2236-2250 and 2254-2277 wild-type plasmid standard, Fig.A3 shows the amplification curve of EGFR Exon 21 position 2573T wild-type plasmid standard, Fig.A4 shows the amplification curve of EGFR Exon 18 position 2155 G→A mutant plasmid standard, Fig.A5 shows the amplification curve of EGFR Exon 19 position 2235-2249 deletion plasmid standard, Fig.A6 shows the amplification curve of EGFR Exon 19 position 2236-2250 deletion plasmid standard, Fig.A7 shows the amplification curve of EGFR Exon 19 position 2254-2277 deletion plasmid standard, Fig.A8 shows the amplification curve of EGFR Exon 21 position 2573 T→G mutant plasmid standard, Fig.B1 shows the amplification curve of KRAS wild-type plasmid standard, Fig.B2 shows the amplification curve of KRAS Codon 12 GGT→GTT mutant plasmid standard, Fig.B3 shows the amplification curve of KRAS Codon 12 GGT→AGT mutant plasmid standard, Fig.B4 shows the amplification curve of KRAS Codon 12 GGT→GAT mutant plasmid standard, Fig.B5 shows the amplification curve of KRAS Codon 12 GGT→TGT mutant plasmid standard, Fig.B6 shows the amplification curve of KRAS Codon 13 GGC→GAC mutant plasmid standard, Fig.C1 shows the amplification curve of BCRP wild-type plasmid standard, Fig.C2 shows the amplification curve of BCRP Codon 482 AGG→GGG mutant plasmid standard, Fig.C3 shows the amplification curve of BCRP Codon 482 AGG→ACG mutant plasmid standard, Fig.D 1 shows the amplification curve of BRAF wild-type plasmid standard, Fig.D2 shows the amplification curve of BRAF Codon 600 GTG→GAG mutant plasmid standard.

FIG. 31 shows the standard curves based on FIG. 30. Fig.A1 shows the standard curve of EGFR Exon 18 position 2155G wild-type plasmid standard, Fig.A2 shows the standard curve of EGFR Exon 19 position 2235-2249, 2236-2250 and 2254-2277 wild-type plasmid standard, Fig.A3 shows the standard curve of EGFR Exon 21 position 2573T wild-type plasmid standard, Fig.A4 shows the standard curve of EGFR Exon 18 position 2155 G→A mutant plasmid standard, Fig.A5 shows the standard curve of EGFR Exon 19 position 2235-2249 deletion plasmid standard, Fig.A6 shows the standard curve of EGFR Exon 19 position 2236-2250 deletion plasmid standard, Fig.A7 shows the standard curve of EGFR Exon 19 position 2254-2277 deletion plasmid standard, Fig.A8 shows the standard curve of EGFR Exon 21 position 2573 T→G mutant plasmid standard, Fig.B 1 shows the standard curve of KRAS wild-type plasmid standard, Fig.B2 shows the standard curve of KRAS Codon 12 GGT→GTT mutant plasmid standard, Fig.B3 shows the standard curve of KRAS Codon 12 GGT→AGT mutant plasmid standard, Fig.B4 shows the standard curve of KRAS Codon 12 GGT→GAT mutant plasmid standard, Fig.B5 shows the standard curve of KRAS Codon 12 GGT→TGT mutant plasmid standard, Fig.B6 shows the standard curve of KRAS Codon 13 GGC→GAC mutant plasmid standard, Fig.C1 shows the standard curve of BCRP wild-type plasmid standard, Fig.C2 shows the standard curve of BCRP Codon 482 AGG→GGG mutant plasmid standard, Fig.C3 shows the standard curve of BCRP Codon 482 AGG→ACG mutant plasmid standard, Fig.D1 shows the standard curve of BRAF wild-type plasmid standard, Fig.D2 shows the standard curve of BRAF Codon 600 GTG→GAG mutant plasmid standard.

FIG. 32 shows the amplification curves of fluorescent quantitative PCR for EGFR Exon 21 position 2573 wild-type (Fig.A1) and T→G replacement (Fig.A2) in tissue sample; fluorescent quantitative PCR for EGFR Exon 19 wild-type (Fig.A3) and position 2235-2249 deletion (Fig.A4) in tissue sample; fluorescent quantitative PCR for EGFR Exon 19 position 2236-2250 deletion (Fig.A5) in whole blood; fluorescent quantitative PCR for EGFR position 2254-2277 deletion (Fig.A6) in whole blood sample; fluorescent quantitative PCR for EGFR Exon 18 position 2155 wild-type (Fig.A7) and G→A replacement (Fig.A8) in cell line sample; fluorescent quantitative PCR for KRAS Codon 12 wild-type (Fig.B1) and GGT→TGT mutant (Fig.B2) in paraffin embedded tissues; fluorescent quantitative PCR for KRAS Codon 12 wild-type (Fig.B3) and GGT→GTT mutant (Fig.B4) in fresh tissue; fluorescent quantitative PCR for KRAS Codon 13 wild-type (Fig.B5) and GGC→GAC mutant (Fig.B6) in whole blood; fluorescent quantitative PCR for KRAS Codon 12 wild-type (Fig.B7) and GGT→AGT mutant (Fig.B8) in cell line sample; fluorescent quantitative PCR for BCRP Codon 482 wild-type (Fig.C1) and AGG→GGG mutant (Fig.C2) in paraffin embedded tissue sample; fluorescent quantitative PCR for BCRP Codon 482 wild-type (Fig.C3) and AGG→ACG mutant (Fig.C4) in fresh tissue sample; fluorescent quantitative PCR for BCRP Codon 482 wild-type (Fig.C5) and AGG→ACG mutant (Fig.C6) in whole blood sample; fluorescent quantitative PCR for BCRP Codon 482 wild-type (Fig.C7) and AGG→GGG mutant (Fig.C8) in cell line sample; fluorescent quantitative PCR for BCRP Codon 482 wild-type (Fig.C5) and AGG→ACG mutant (Fig.C6) in whole blood sample; fluorescent quantitative PCR for BRAF Codon 600 wild-type (Fig.D1) and GTG→GAG mutant (Fig.D2) in paraffin embedded tissue sample; fluorescent quantitative PCR for BRAF Codon 600 wild-type (Fig.D3) and GTG→GAG mutant (Fig.D4) in fresh tissue sample; fluorescent quantitative PCR for BRAF Codon 600 wild-type (Fig.D5) and GTG→GAG mutant (Fig.D6) in whole blood sample; fluorescent quantitative PCR for BRAF Codon 600 wild-type (Fig.D7) and GTG→GAG mutant (Fig.D8) in cell line sample; detected in Example 25,

FIG. 33 is a diagram showing the amplification curve of the plasmid standard of Example 26. Fig.A1 shows the amplification curve of ERCC1 plasmid standard, Fig.A2 shows the amplification curve of RRM1 plasmid standard, Fig.A3 shows the amplification curve of BRCA1 plasmid standard, Fig.A4 shows the amplification curve of TUBB3 plasmid standard, Fig.A5 shows the amplification curve of ERBB3 plasmid standard, Fig.A6 shows the amplification curve of TOP2A plasmid standard, Fig.A7 shows the amplification curve of TYMS plasmid standard, Fig.A1 shows the amplification curve of ERCC1 plasmid standard, Fig.A8 shows the amplification curve of RAP-80 plasmid standard, Fig.A9 shows the amplification curve of VEGFR1 plasmid standard, Fig.A10 shows the amplification curve of VEGFR2 plasmid standard, Fig.A11 shows the amplification curve of HER2 plasmid standard, Fig.A12 shows the amplification curve of EGFR plasmid standard, Fig.A13 shows the amplification curve of VEGF plasmid standard, Fig.A14 shows the amplification curve of PPN plasmid standard, Fig.A15 shows the amplification curve of CCNB2 plasmid standard, Fig.A16 shows the amplification curve of ACTB plasmid standard, Fig.A17 shows the amplification curve of 18S rRNA plasmid standard.

FIG. 34 shows the standard curves based on FIG. 33. Fig.A1 shows the standard curve of ERCC1 plasmid standard, Fig.A2 shows the standard curve of RRM1 plasmid standard, Fig.A3 shows the standard curve of BRCA1 plasmid standard, Fig.A4 shows the standard curve of TUBB3 plasmid standard, Fig.A5 shows the standard curve of ERBB3 plasmid standard, Fig.A6 shows the standard curve of TOP2A plasmid standard, Fig.A7 shows the standard curve of TYMS plasmid standard, Fig.A1 shows the standard curve of ERCC1 plasmid standard, Fig.A8 shows the standard curve of RAP-80 plasmid standard, Fig.A9 shows the standard curve of VEGFR1 plasmid standard, Fig.A10 shows the standard curve of VEGFR2 plasmid standard, Fig.A11 shows the standard curve of HER2 plasmid standard, Fig.A12 shows the standard curve of EGFR plasmid standard, Fig.A13 shows the standard curve of VEGF plasmid standard, Fig.A14 shows the standard curve of PPN plasmid standard, Fig.A15 shows the standard curve of CCNB2 plasmid standard, Fig.A16 shows the standard curve of ACTB plasmid standard, Fig.A17 shows the standard curve of 18S rRNA plasmid standard.

FIG. 35 is a diagram showing the amplification curve of the samples detected in Example 26, which are, from left to right, 18S rRNA, ACTB, ERCC1, TYMS, RRM1, BRCA1, TUBB3, TOP2A, PPN, VEGF, EGFR, CCNB2, VEGFR1, RAP-80, HER2, ERBB3, VEGFR2 genes.

FIG. 36 is a diagram showing the amplification curve of the standard of Example 27, wherein Figure A shows the amplification curve of HER2 plasmid standard, Figure B shows the amplification curve of ACTB plasmid standard.

FIG. 37 shows the standard curves based on FIG. 36. Fig.A1 shows the standard curve of HER2 plasmid standard, Fig.B shows the standard curve of ACTB plasmid.

FIG. 38 is a diagram showing the gene amplification curve of the sample of Example 27, wherein Figure A shows the amplification curve of HER2 in paraffin embedded tissue, Figure B shows the amplification curve of HER2 in fresh tissue, Figure C shows the amplification curve of ACTB in paraffin embedded tissue, Figure D shows the amplification curve of ACTB in fresh tissue.

### DETAILED DESCRIPTION OF PARTICULAR EMBODIMENTS

### Examples

The following examples are put forth so as to provide those of ordinary skill in the art with a complete disclosure and description of how to make use of the present invention, and are neither intended to limit the scope of what the inventors regard as their invention nor are they intended to represent that the experiments below are all or the only experiments performed. The experimental conditions not indicated in the Examples, are generally conventional, such as those disclosed in “Molecular Cloning, A Laboratory Manual, 3^{rd} ed, (Sambrook J.)”, or those suggested by the manufacturer.

### Example 1

### Extraction and Preparation of Nucleic Acid from Human Cell Lines, Fresh Human Tumor Tissues, Peripheral Blood, and Paraffin Embedded Tissues

1. Extraction of DNA or RNA

Nucleic acid extracting kit from Qiagen Inc., Promega Inc., or Roche Inc. can be used to extract nucleic acid from the samples. Content and purity of the extracted nucleic acid can be determined by using Nanodrop ND 1000 (Gene Inc.):

DNA: OD260/OD280=1.8±0.1, OD260/OD230=2.0±0.1;

RNA: OD260/OD280=2.0±0.1, OD260/OD230=2.0±0.10

2. Synthesis of cDNA

Use M-MLV reverse transcriptase to perform reverse transcription. The steps and reaction system are as in Table 1:

TABLE 1
reverse transcription system (10 μl) and steps

| reagent | amount (μl/tube) |
|---|---|
| RNA template | 5.5 |
| OligodT | 0.4 |
| 70° C. denature 5 min, ice bath 2-5 min, add the following: | |
| 5 X buffer | 2 |
| dNTP(5 mM) | 1 |
| DEPC water | 0.35 |
| RNasin(40 U) | 0.25 |
| MLV RT-enzyme | 0.5 |
| total | 10 |

37-42° C. 60-90 min, 70° C. 5 min.

### Example 2

### Preparation of Positive Standard for EGFR Mutant Test

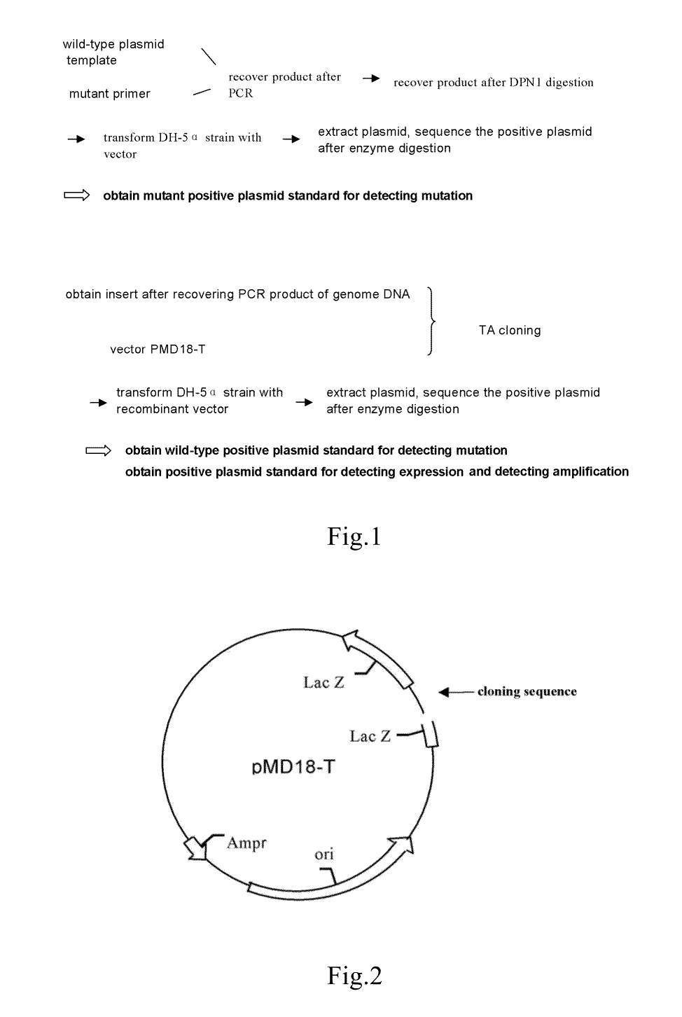

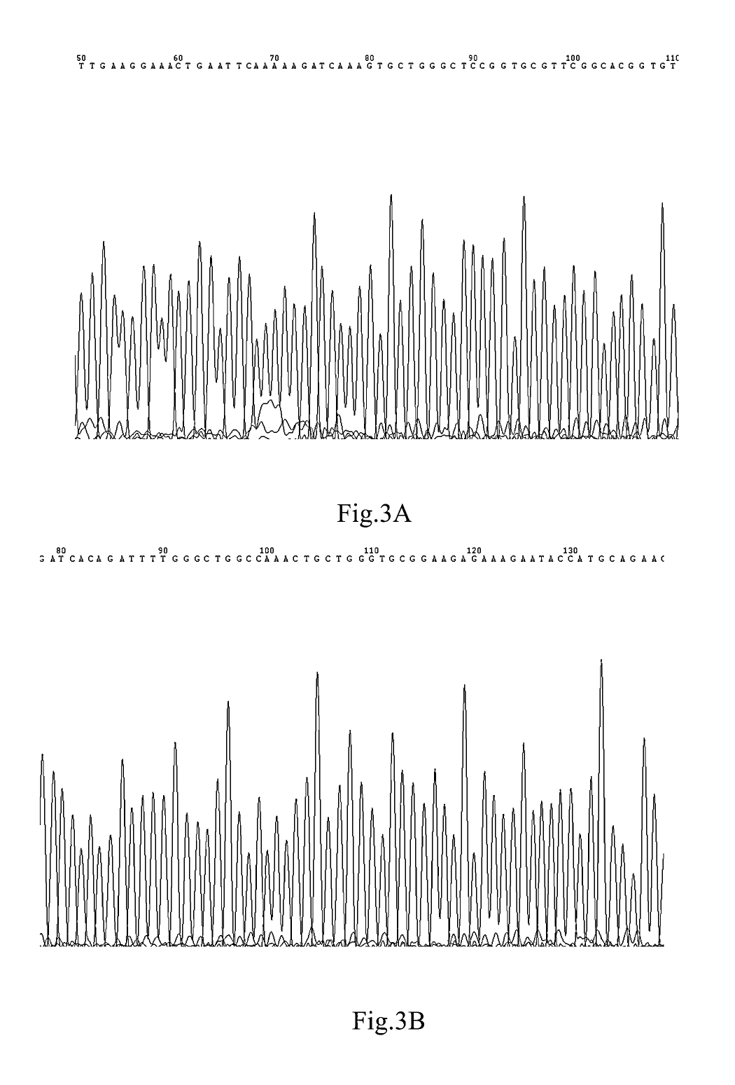

1. Construction of wild-type plasmids (FIG. 1, FIG. 2)

1.1 Preparation of the Vector

TA cloning vector pMD18-T was purchased from TAKARA Inc.

1.2 Preparation of the Insert

The insert is prepared using PCR. The template of PCR is the sample genome DNA extracted in Example 1. The reaction system and amplification condition are shown in the following tables (Table 2, Table 3 and Table 4):

TABLE 2
PCR reaction system (50 μl)

| reagents | amount(μl/tube) |
|---|---|
| double-distilled water | 29.75 |
| 10x buffer (free of Mg²⁺) | 5 |
| MgCl₂ (25 mM) | 7.5 |
| dNTP (10 mM) | 1.25 |
| upstream primer (25 μM) | 1.25 |
| downstream primer (25 μM) | 1.25 |
| Taq enzyme | 1 |
| DNA template | 3 |
| total volume | 50 |

TABLE 3
PCR amplification condition

| step | cycles | temperature and time |
|---|---|---|
| step 1 | 1 | 95° C., 1-5 minutes |
| step 2 | 20-30 | 95° C., 10-15 seconds; 55-65° C., 30-60 seconds |

TABLE 4
PCR primers

| name | sequence | |
|---|---|---|
| E18-F1 | GAGGATCTTGAAGGAAACTG | (SEQ ID NO: 2) |
| E18-F2 | CCAGCTTGTGGAGCCTCTT | (SEQ ID NO: 3) |
| E18-R1 | GCCAGGGACCTTACCTTAT | (SEQ ID NO: 4) |
| E18-R2 | CTGTGCCAGGGACCTTACCTT | (SEQ ID NO: 5) |
| E18-F1 | CCCAGAAGGTGAGAAAGTT | (SEQ ID NO: 6) |
| E18-F2 | GGGACTCTGGATCCCAGAAG | (SEQ ID NO: 7) |
| E18-R1 | CCTGAGGTTCAGAGCCAT | (SEQ ID NO: 8) |
| E18-R2 | CCCACACAGCAAAGCAGAA | (SEQ ID NO: 9) |
| E21-F1 | GCAGCCAGGAACGTACTGGT | (SEQ ID NO: 10) |
| E21-F2 | CCCTCACAGCAGGGTCTTCT | (SEQ ID NO: 11) |
| E21-R1 | GTGGGAAGGCAGCCTGGT | (SEQ ID NO: 12) |
| E21-R2 | GTGGGAAGGCAGCCTGGT | (SEQ ID NO: 13) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

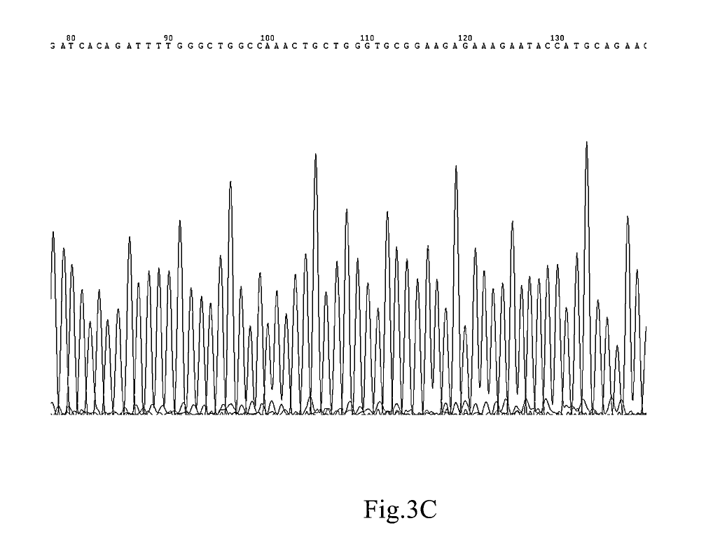

1.6 Sequence the strains having positive result, and use the strains with correct sequence as the standard containing wild-type sequence (FIG. 3).

2. Construction of mutant plasmids: design mutant primers of mutant sites, obtain the standards containing mutant sequences by DPN1 method.

2.1 Design the mutant primers (Table 5) of mutant sites based on the desired mutant sequences.

TABLE 5
mutant primers

| primers name | sequence | |
|---|---|---|
| E18-M-F: | TGCTGAGCTCCGGTGCGTTCG | (SEQ ID NO: 14) |
| E18-M-R: | GGAGCTCAGCACTTTGATCTT | (SEQ ID NO: 15) |
| E19-1-F: | ATCAAAACATCTCCGAAAGCC | (SEQ ID NO: 16) |
| E19-1-R: | ATGTTTTGATAGCGACGGGAA | (SEQ ID NO: 17) |
| E19-2-F: | TCAAGACATCTCCGAAAGCCA | (SEQ ID NO: 18) |
| E19-2-R: | GATGTCTTGATAGCGACGGGA | (SEQ ID NO: 19) |
| E19-3-F: | CAACACTCGATGTGAGTTTCT | (SEQ ID NO: 20) |
| E19-3-R: | TCGAGTGTTGCTTCTCTTAAT | (SEQ ID NO: 21) |
| E21-M-F: | TGGGCGGGCCAAACTGCTGGG | (SEQ ID NO: 22) |
| E21-M-R: | TGGCCCGCCCAAAATCTGTGA | (SEQ ID NO: 23) |

2.2 Use 5 ng wild-type plasmid as template, and use mutant primers and Pfu enzyme to mutate the target sites. The amplification system and condition are shown in Table 2, Table 3 and Table 5.

During the preparation of the plasmid containing EGFR Exon 18 position 2155 G→A mutant sequence, the amplification system needs to add E18-M-F (SEQ ID NO:14) and E18-M-R (SEQ ID NO:15) primers. During the preparation of the plasmid containing EGFR Exon 19 position 2235-2249 deletion sequence, the amplification system needs to add E19-1-F (SEQ ID NO:16) and E19-1-R (SEQ ID NO:17) primers. During the preparation of the plasmid containing EGFR Exon 19 position 2236-2250 deletion sequence, the amplification system needs to add E19-2-F (SEQ ID NO:18) and E19-2-R (SEQ ID NO:19) primers. During the preparation of the plasmid containing EGFR Exon 19 position 2254-2277 deletion sequence, the amplification system needs to add E19-3-F (SEQ ID NO:20) and E19-1-R (SEQ ID NO:21) primers. During the preparation of the plasmid containing EGFR Exon 21 position 2573 T→G mutant sequence, the amplification system needs to add E21-M-F (SEQ ID NO:22) and E21-M-R (SEQ ID NO:23) primers.

2.3 treat the product obtained in step 2.2 with DPN1 enzyme, recover the product after incubating at 37° C. for 1 hour, amplify in E. coli DH5α strain, and harvest by extraction and purification.

2.4 Identify the plasmid by double enzyme digestion of BamHI and HindIII.

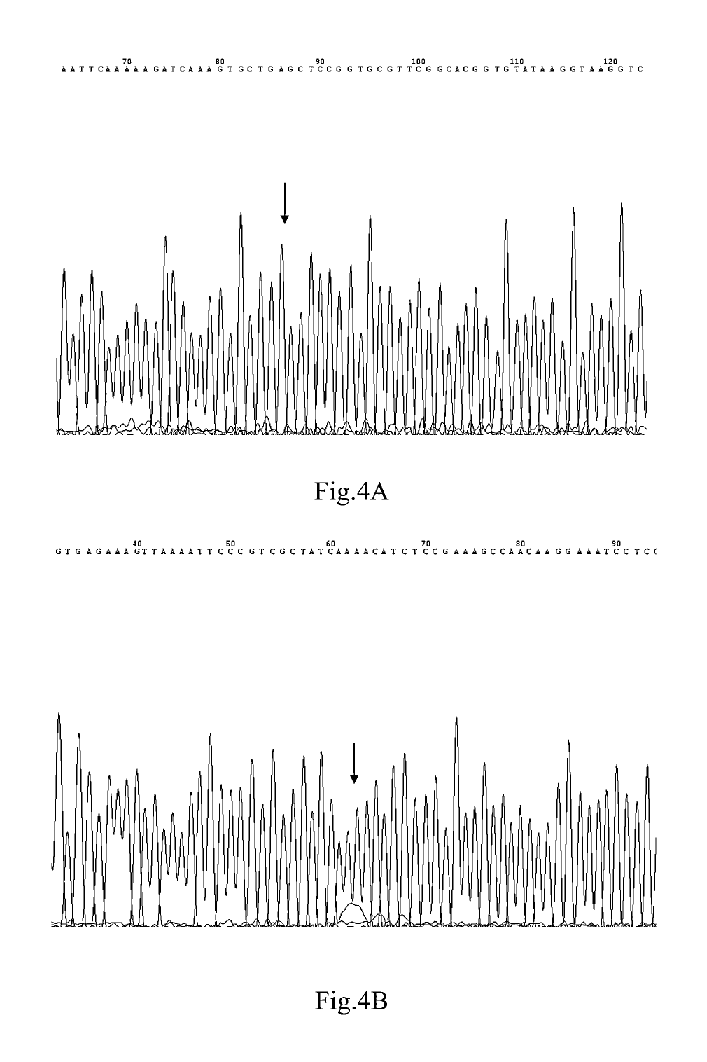

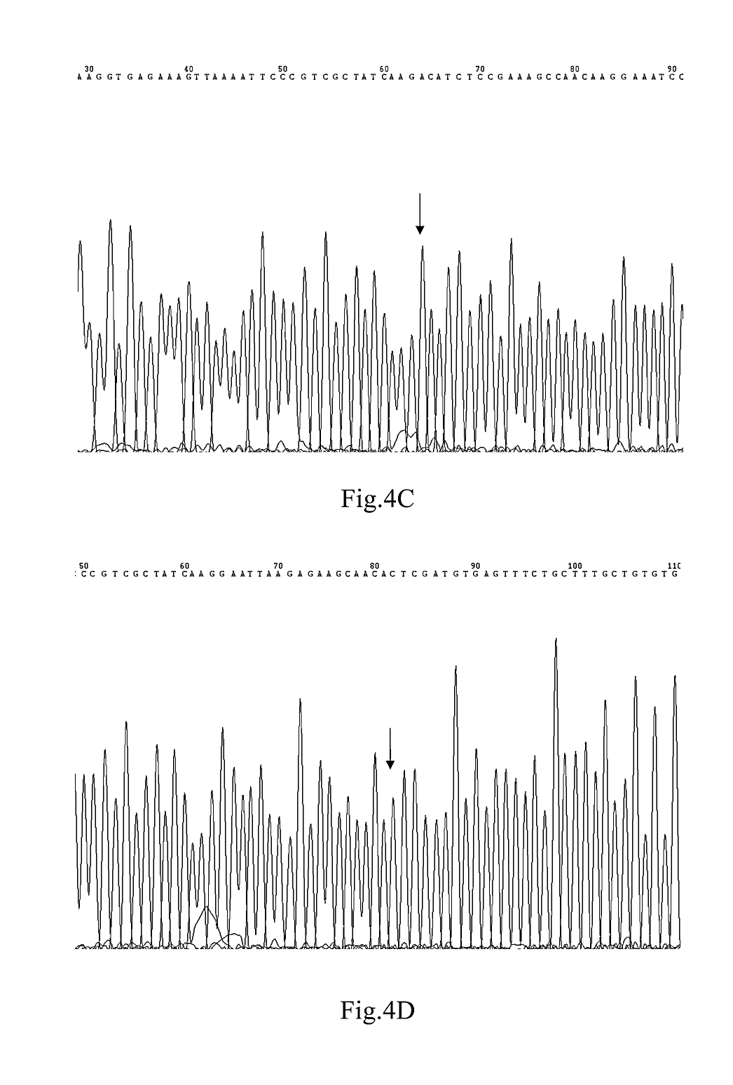

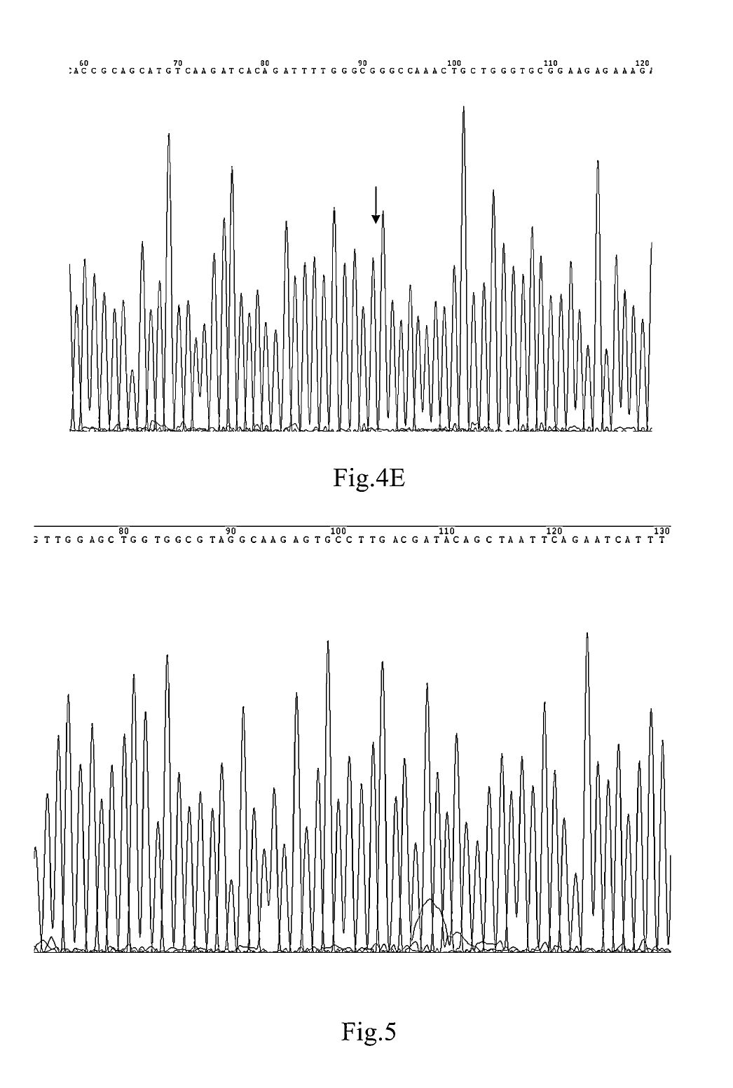

2.5 Sequence the strains having positive result, and use the strains with correct sequence as the standard containing mutant sequence (FIG. 4).

### Example 3

### Preparation of Positive Standard for KRAS Mutant Test

1. Construction of Wild-Type Plasmids (FIG. 1, FIG. 2)

1.1 Preparation of the Vector

TA cloning vector pMD18-T was purchased from TAKARA Inc.

1.2 Preparation of the Insert

The insert is prepared using PCR. The template of PCR is the sample genome DNA extracted in Example 1. The reaction system and amplification condition are shown in the following tables (Table 2, Table 3 and Table 6):

TABLE 6
PCR primers

| name | sequence | |
|---|---|---|
| KRAS-F1 | CCTCTATTGTTGGATCATATT | (SEQ ID NO: 25) |
| KRAS-F2 | AATGACTGAATATAAACTTGTGGTA | (SEQ ID NO: 26) |
| | GT | |
| KRAS-R1 | TGACTGAATATAAACTTGTGGT | (SEQ ID NO: 27) |
| KRAS-R2 | AAATGATTCTGAATTAGCTGTATCGT | (SEQ ID NO: 28) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the standard containing wild-type sequence (FIG. 5).

2. Construction of mutant plasmids: design mutant primers of mutant sites, obtain the standards containing mutant sequences by DPN1 method.

2.1 Design the mutant primers (Table 7) of mutant sites based on the desired mutant sequences.

TABLE 7
mutant primers

| primers name | sequence | |
|---|---|---|
| KRAS-1-F: | AGCTGTTGGCGTAGGCAAGAG | (SEQ ID NO: 29) |
| KRAS-1-R: | CGCCAACAGCTCCAACTACCA | (SEQ ID NO: 30) |
| KRAS-2-F: | AGCTAGTGGCGTAGGCAAGAG | (SEQ ID NO: 31) |
| KRAS-2-R: | CGCCACTAGCTCCAACTACCA | (SEQ ID NO: 32) |
| KRAS-3-F: | AGCTGATGGCGTAGGCAAGAG | (SEQ ID NO: 33) |
| KRAS-3-R: | CGCCATCAGCTCCAACTACCA | (SEQ ID NO: 34) |
| KRAS-4-F: | AGCTTGTGGCGTAGGCAAGAG | (SEQ ID NO: 35) |
| KRAS-4-R: | CGCCACAAGCTCCAACTACCA | (SEQ ID NO: 36) |
| KRAS-5-F: | CTGGTGACGTAGGCAAGAGTG | (SEQ ID NO: 37) |
| KRAS-5-R: | CCTACGTCACCAGCTCCAACT | (SEQ ID NO: 38) |

2.2. Use 5 ng wild-type plasmid as template, and use mutant primers and Pfu enzyme to mutate the target sites. The amplification system and condition are shown in Table 2, Table 3 and Table 7.

During the preparation of the plasmid containing KRAS Codon 12 GGT→GTT mutant sequence, the amplification system needs to add KRAS-1-F (SEQ ID NO:29) and KRAS-1-R (SEQ ID NO:30) primers. During the preparation of the plasmid containing KRAS Codon 12 GGT→AGT mutant sequence, the amplification system needs to add KRAS-2-F (SEQ ID NO:31) and KRAS-2-R (SEQ ID NO:32) primers. During the preparation of the plasmid containing KRAS Codon 12 GGT→GAT mutant sequence, the amplification system needs to add KRAS-3-F (SEQ ID NO:33) and KRAS-3-R (SEQ ID NO:34) primers. During the preparation of the plasmid containing KRAS Codon 12 GGT→TGT mutant sequence, the amplification system needs to add KRAS-4-F (SEQ ID NO:35) and KRAS-4-R (SEQ ID NO:36) primers. During the preparation of the plasmid containing KRAS Codon 13 GGC→GAC mutant sequence, the amplification system needs to add KRAS-5-F (SEQ ID NO:37) and KRAS-5-R (SEQ ID NO:38) primers.

2.3 treat the product obtained in step 2.2 with DPN1 enzyme, recover the product after incubating at 37° C. for 1 hour, amplify in E. coli DH5α strain, and harvest by extraction and purification.

2.4 Identify the plasmid by double enzyme digestion of BamHI and HindIII.

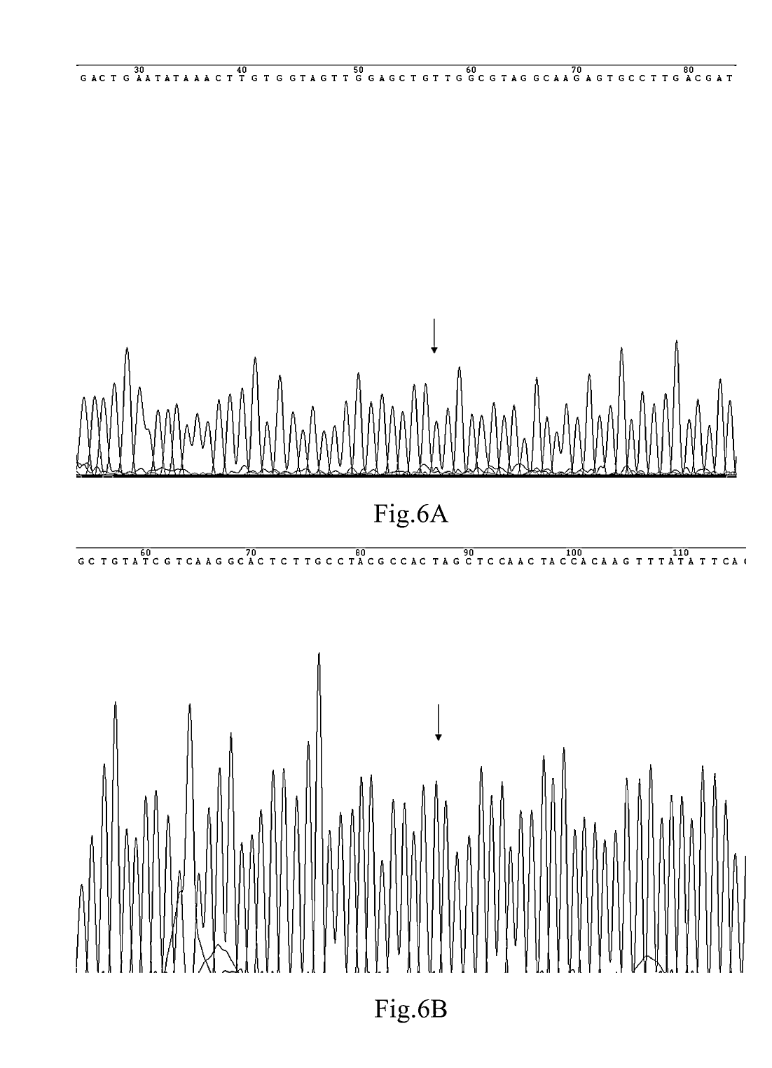

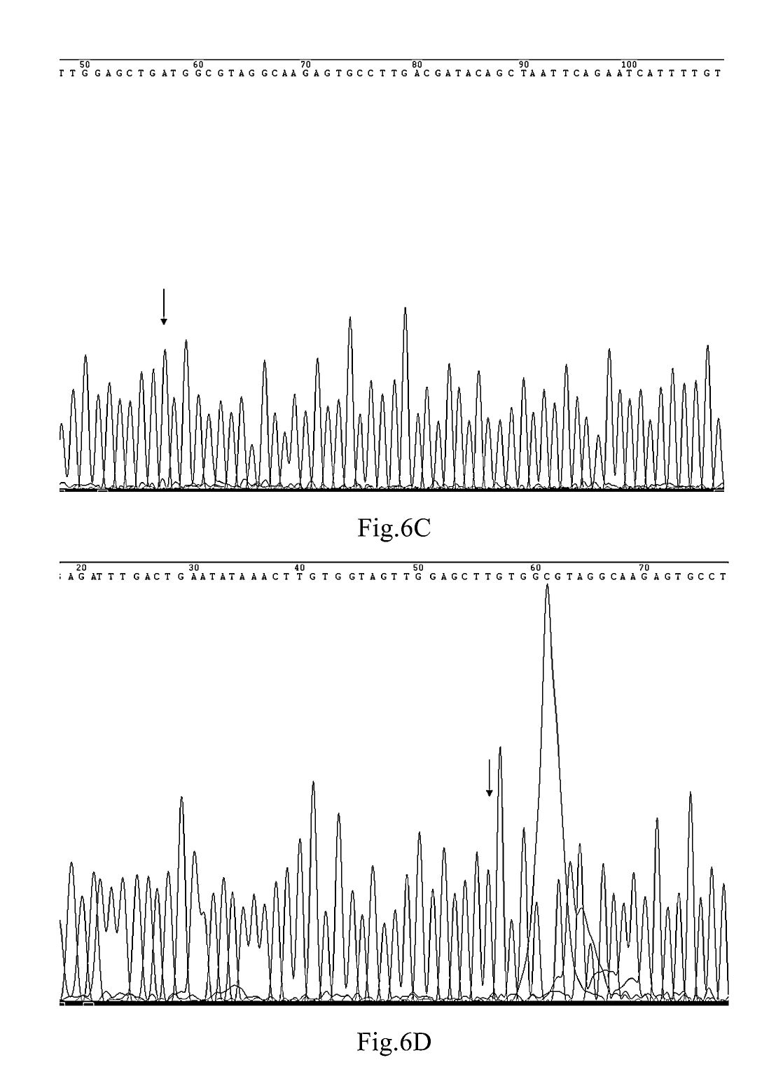

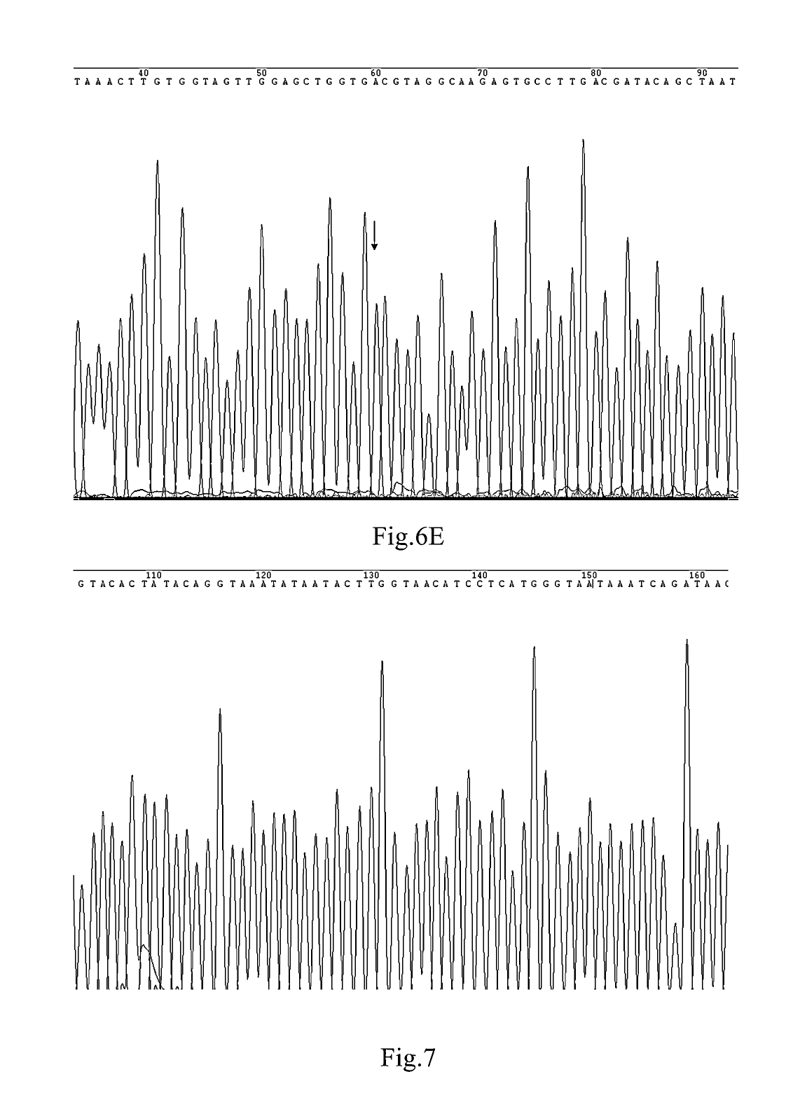

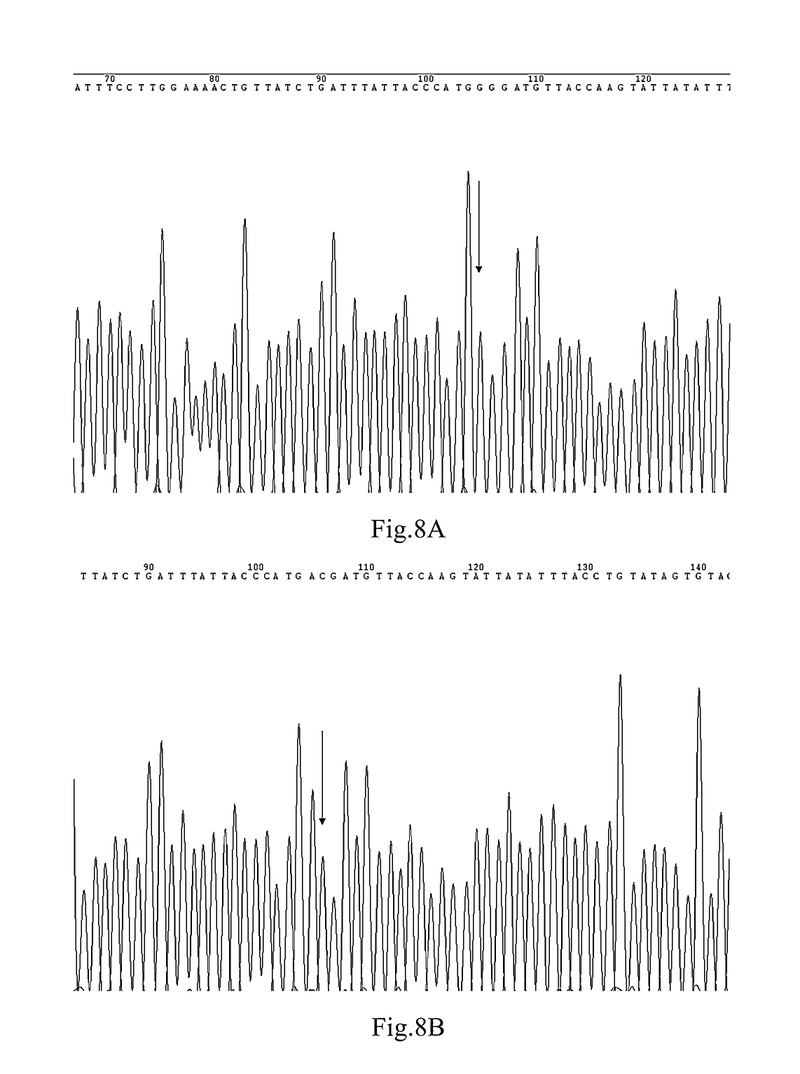

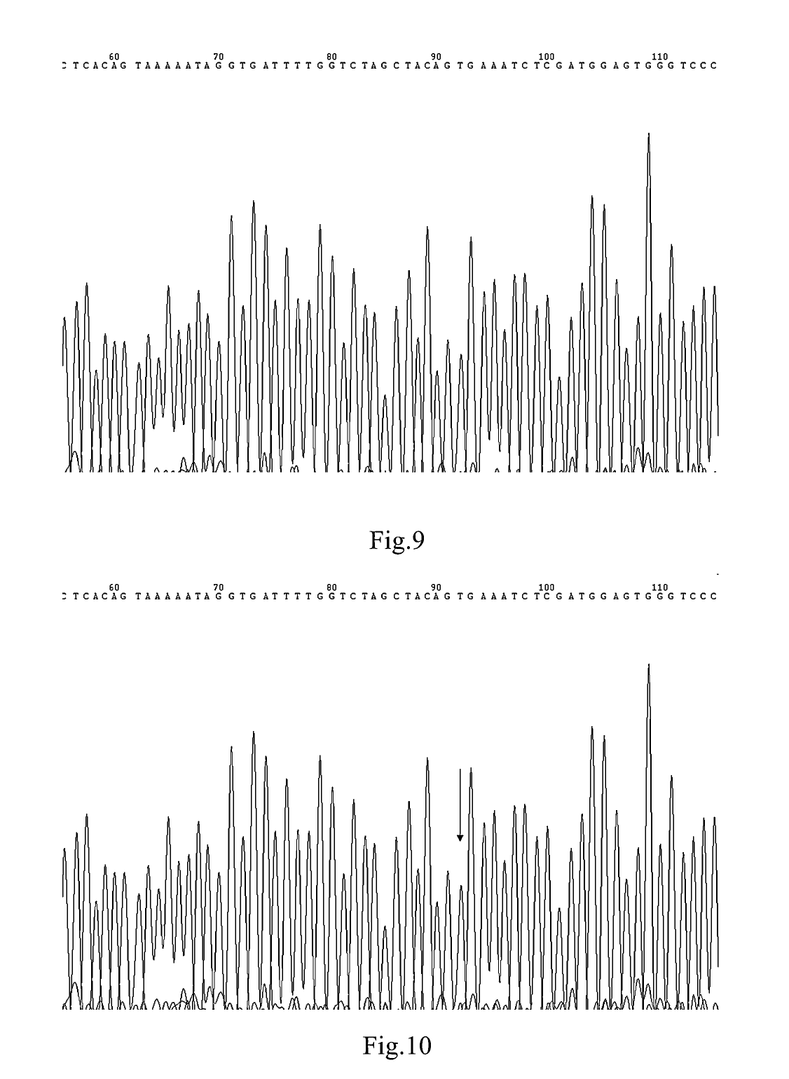

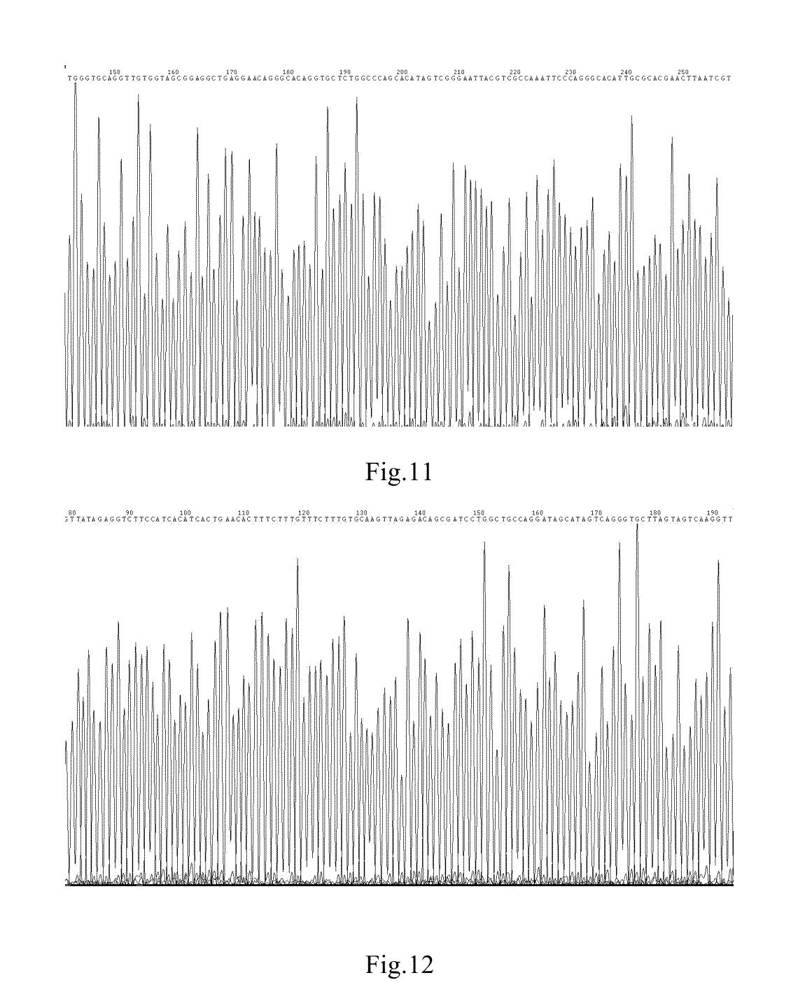

2.5 Sequence the strains having positive result, and use the strains with correct sequence as the standard containing mutant sequence (FIG. 6).

### Example 4

### Preparation of Positive Standard for BCRP Mutant Test

1. Construction of Wild-Type Plasmids (FIG. 1, FIG. 2)

1.1 Preparation of the Vector

TA cloning vector pMD18-T was purchased from TAKARA Inc.

1.2 Preparation of the Insert

The insert is prepared using PCR. The template of PCR is the sample genome DNA extracted in Example 1. The reaction system and amplification condition are shown in the following tables (Table 2, Table 3 and Table 8):

TABLE 8
PCR primers

| name | sequences | |
|---|---|---|
| BCRP-F1 | CTACAGAGTGTCATCTTATTTCCT | (SEQ ID NO: 40) |
| BCRP-F2 | TCCTTGGAAAACTGTTATCTGAT | (SEQ ID NO: 41) |
| BCRP-F3 | GCGGATACTACAGAGTGTCAT | (SEQ ID NO: 42) |
| BCRP-R1 | CATGAAGTACACTATACAGGTAA | (SEQ ID NO: 43) |
| | ATA | |
| BCRP-R2 | TAACATGAAGTACACTATACAGG | (SEQ ID NO: 44) |
| | TA | |
| BCRP-R3 | GGCAAGACTAAAGACATGTCC | (SEQ ID NO: 45) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

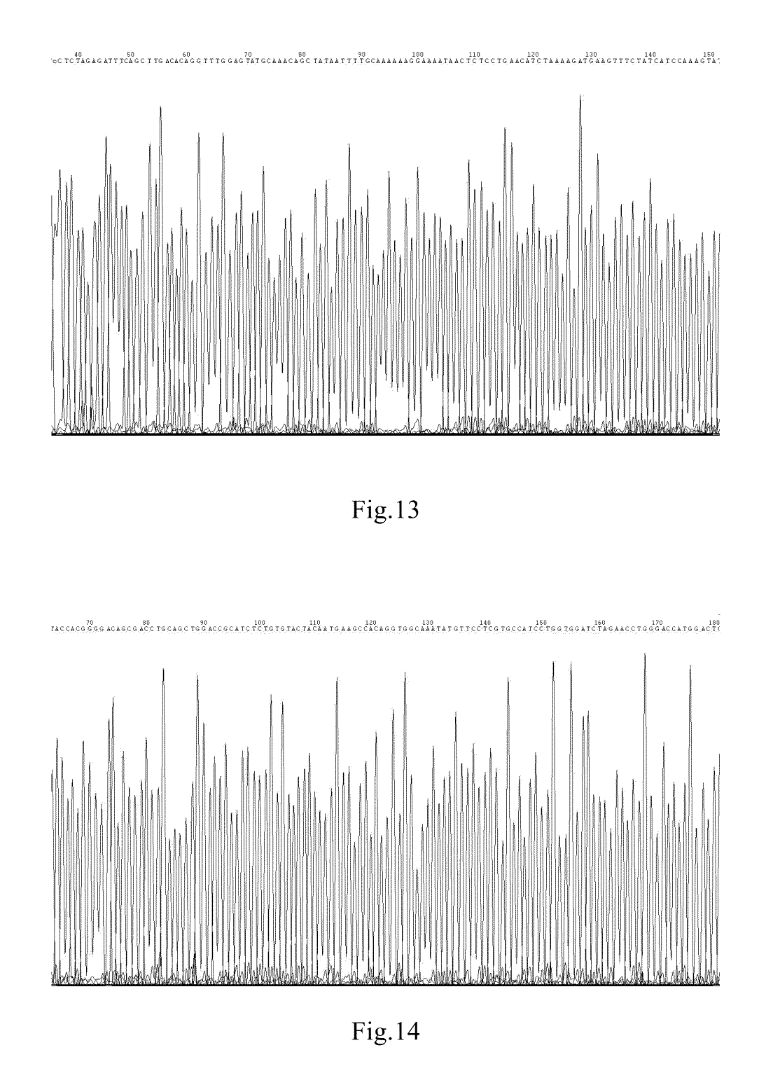

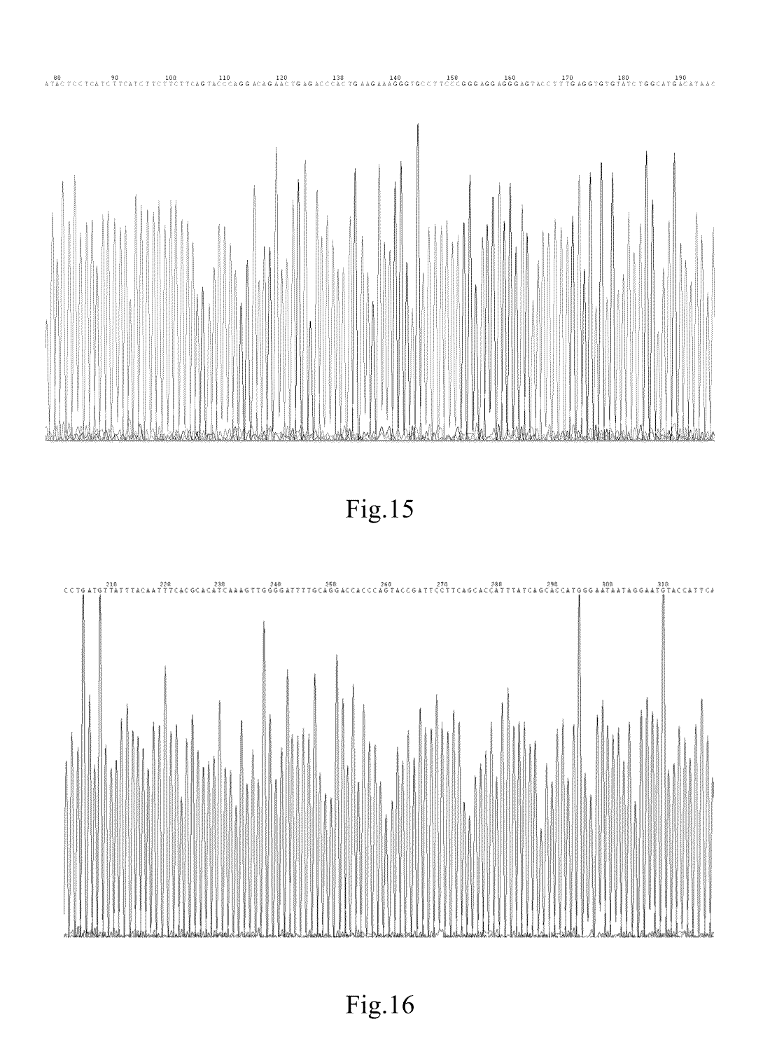

1.6 Sequence the strains having positive result, and use the strains with correct sequence as the standard containing wild-type sequence (FIG. 7).

2. Construction of mutant plasmids: design mutant primers of mutant sites, obtain the standards containing mutant sequences by DPN1 method.

2.1 Design the mutant primers (Table 9) of mutant sites based on the desired mutant sequences.

TABLE 9
mutant primers

| primers name | sequence | |
|---|---|---|
| BCRP-1-F: | CCATGGGGATGTTACCAAGTA | (SEQ ID NO: 46) |
| BCRP-1-R: | CATCCCCATGGGTAATAAATC | (SEQ ID NO: 47) |
| BCRP-2-F: | CCATGACGATGTTACCAAGTA | (SEQ ID NO: 48) |
| BCRP-2-R: | CATCGTCATGGGTAATAAATC | (SEQ ID NO: 49) |

2.2 Use 5 ng wild-type plasmid as template, and use mutant primers and

Pfu enzyme to mutate the target sites. The amplification system and condition are shown in Table 2, Table 3 and Table 9.

During the preparation of the plasmid containing BCRP Codon 482 AGG→GGG mutant sequence, the amplification system needs to add BCRP-1-F (SEQ ID NO:46) and BCRP-1-R (SEQ ID NO:47) primers. During the preparation of the plasmid containing BCRP Codon 482 AGG→ACG mutant sequence, the amplification system needs to add BCRP-2-F (SEQ ID NO:48) and BCRP-2-R (SEQ ID NO:49) primers.

2.3 treat the product obtained in step 2.2 with DPN1 enzyme, recover the product after incubating at 37° C. for 1 hour, amplify in E. coli DH5α strain, and harvest by extraction and purification.

2.4 Identify the plasmid by double enzyme digestion of BamHI and HindIII.

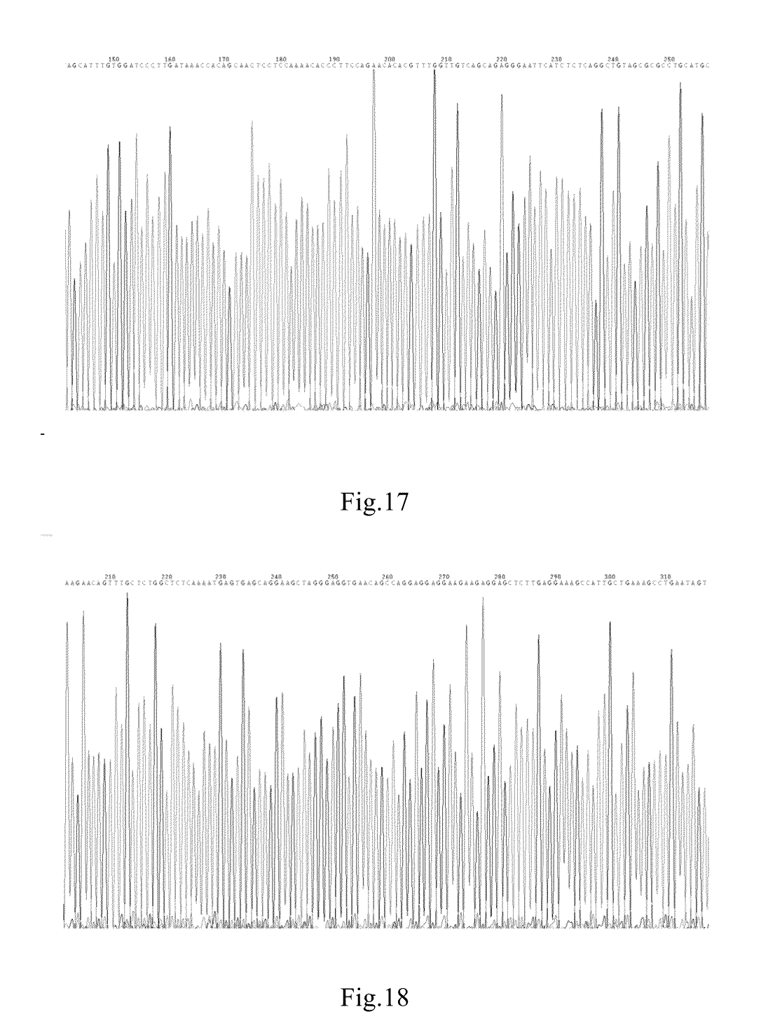

2.5 Sequence the strains having positive result, and use the strains with correct sequence as the standard containing mutant sequence (FIG. 8).

### Example 5

### Preparation of Positive Standard for BRAF Mutant Test

1. Construction of Wild-Type Plasmids (FIG. 1, FIG. 2)

1.1 Preparation of the Vector

TA cloning vector pMD18-T was purchased from TAKARA Inc.

1.2 Preparation of the Insert

The insert is prepared using PCR. The template of PCR is the sample genome DNA extracted in Example 1. The reaction system and amplification condition are shown in the following tables (Table 2, Table 3 and Table 10):

TABLE 10
PCR primers

| name | sequence | |
|---|---|---|
| BRAF-F1 | CATGAAGACCTCACAGTAAAAAT | (SEQ ID NO: 51) |
| | AGGTGAT | |
| BRAF-F2 | TTCTTCATGAAGACCTCACAGTAA | (SEQ ID NO: 52) |
| BRAF-R1 | GGATCCAGACAACTGTTCAAACT | (SEQ ID NO: 53) |
| | GA | |
| BRAF-R2 | CCAGACAACTGTTCAAACTGATG | (SEQ ID NO: 54) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the standard containing wild-type sequence (FIG. 9).

2. Construction of mutant plasmids: design mutant primers of mutant sites, obtain the standards containing mutant sequences by DPN1 method.

2.1 Design the mutant primers (Table 11) of mutant sites based on the desired mutant sequences.

TABLE 11
mutant primers

| name | sequence | |
|---|---|---|
| BRAF-1-F: | TACAGAGAAATCTCGATGGAG | (SEQ ID NO: 55) |
| BRAF-1-R: | ATTTCTCTGTAGCTAGACCAA | (SEQ ID NO: 56) |

2.2 Use 5 ng wild-type plasmid as template, and use mutant primers and Pfu enzyme to mutate the target sites. The amplification system and condition are shown in Table 2, Table 3 and Table 11.

During the preparation of the plasmid containing BRAF Codon 600 GTG→GAG mutant sequence, the amplification system needs to add BRAF-1-F (SEQ ID NO:55) and BRAF-1-R (SEQ ID NO:56) primers.

2.3 treat the product obtained in step 2.2 with DPN1 enzyme, recover the product after incubating at 37° C. for 1 hour, amplify in E. coli DH5α strain, and harvest by extraction and purification.

2.4 Identify the plasmid by double enzyme digestion of BamHI and HindIII.

2.5 Sequence the strains having positive result, and use the strains with correct sequence as the standard containing mutant sequence (FIG. 10).

### Example 6

### Preparation of ERCC1 Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 12):

TABLE 12
PCR primers

| name | sequence | |
|---|---|---|
| ERCC1-F-1 | GAAAGAGACGGAGCTGAGGAA | (SEQ ID NO: 57) |
| ERCC1-F-2 | GGGAATTTGGCGACGTAATTC | (SEQ ID NO: 58) |
| ERCC1-R-1 | GGCCCTGACCTTGTAGACTGT | (SEQ ID NO: 59) |
| ERCC1-R-2 | GCGGAGGCTGAGGAACAG | (SEQ ID NO: 60) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 11).

### Example 7

### Preparation of positive standard for RRM1 (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 13):

TABLE 13
PCR primers

| name | sequence | |
|---|---|---|
| RRM1-F-1 | TGACTACTAAGCACCCTGACTATG | (SEQ ID NO: 61) |
| RRM1-F-2 | ACCCACCAGTCAAAGC | (SEQ ID NO: 62) |
| RRM1-R-1 | CTTCCATCACATCACTGAACACTT | (SEQ ID NO: 63) |
| RRM1-R-2 | CATACAGGGAGTGGTTAAGT | (SEQ ID NO: 64) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 12).

### Example 8

### Preparation of BRCA1 Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 14):

TABLE 14
PCR primers

| name | sequence | |
|---|---|---|
| BRCA1-F-1 | TCATCCAAAGTATGGGCTACAGA | (SEQ ID NO: 65) |
| | TGACTATG | |
| BRCA1-F-2 | TCATCCAAAGTATGGGCTACAGA | (SEQ ID NO: 66) |
| BRCA1-R-1 | TGGACACTGAGACTGGTTTCC | (SEQ ID NO: 67) |
| BRCA1-R-2 | TGGACACTGAGACTGGTTTC | (SEQ ID NO: 68) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 13).

### Example 9

### Preparation of Positive Standard for TUBB3 (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 15):

TABLE 15
PCR primers

| name | sequence | |
|---|---|---|
| TUBB3-F-1 | GACCGCATCTCTGTGTACTAC | (SEQ ID NO: 69) |
| TUBB3-F-2 | CTGTGTACTACAATGAAGCCAC | (SEQ ID NO: 70) |
| TUBB3-R-1 | GTCCATGGTCCCAGGTTCTA | (SEQ ID NO: 71) |
| TUBB3-R-2 | AGGTTCTAGATCCACCAGGATGG | (SEQ ID NO: 72) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 14).

### Example 10

### Preparation of ERBB3 Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 16):

TABLE 16
PCR primers

| name | sequence | |
|---|---|---|
| ERBB3-F-1 | CAACGGTTATGTCATGCCAGA | (SEQ ID NO: 73) |
| ERBB3-F-2 | GGTTATGTCATGCCAGATACAC | (SEQ ID NO: 74) |
| ERBB3-R-1 | GACAGAACTGAGACCCACTGAAG | (SEQ ID NO: 75) |
| ERBB3-R-2 | CTGAGACCCACTGAAGAAAGGGT | (SEQ ID NO: 76) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 15).

### Example 11

### Preparation of TOP2a Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 17):

TABLE 17
PCR primers

| name | sequence | |
|---|---|---|
| TOP2A-F-1 | CAGCGTGTTGAGCCTGAATG | (SEQ ID NO: 77) |
| TOP2A-F-2 | GCGTGTTGAGCCTGAATGGTAC | (SEQ ID NO: 78) |
| TOP2A-R-1 | AGGACCACCCAGTACCGATT | (SEQ ID NO: 79) |
| TOP2A-R-2 | GGACCACCCAGTACCGATTCCT | (SEQ ID NO: 80) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 16).

### Example 12

### Preparation of TYMS Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 18):

TABLE 18
PCR primers

| name | sequence | |
|---|---|---|
| TYMS-F-1 | GGCACCCTGTCGGTATTCG | (SEQ ID NO: 81) |
| TYMS-F-2 | GGCACCCTGTCGGTATTC | (SEQ ID NO: 82) |
| TYMS-R-1 | CCCTTCCAGAACACACGTT | (SEQ ID NO: 83) |
| TYMS-R-2 | CTCCAAAACACCCTTCCAGAA | (SEQ ID NO: 84) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 17).

### Example 13

### Preparation of RAP-80 Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 19):

TABLE 19
PCR primers

| name | sequence | |
|---|---|---|
| RAP-80-F-1 | CAAAATGAGTGAGCAGGAAGCT | (SEQ ID NO: 85) |
| RAP-80-F-2 | AATGAGTGAGCAGGAAGCT | (SEQ ID NO: 86) |
| RAP-80-R-1 | TCAGAAGGCCGGCAACTATT | (SEQ ID NO: 87) |
| RAP80-R-2 | CAACTATTCAGGCTTTCAGCAAT | (SEQ ID NO: 88) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 18).

### Example 14

### Preparation of VEGFR1 Positive Standard (FIG. 1, FIG. 2)

1. Preparation of the Vector TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 20):

TABLE 20
PCR primers

| name | sequence | |
|---|---|---|
| VEGFR1-F-1 | TTTACCGAATGCCACCTCCAT | (SEQ ID NO: 89) |
| VEGFR1-F-2 | CCGAATGCCACCTCCAT | (SEQ ID NO: 90) |
| VEGFR1-R-1 | ATGGGAGAGGCCAACAGAGT | (SEQ ID NO: 91) |
| VEGFR1-R-2 | GGGAGAGGCCAACAGAGT | (SEQ ID NO: 92) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

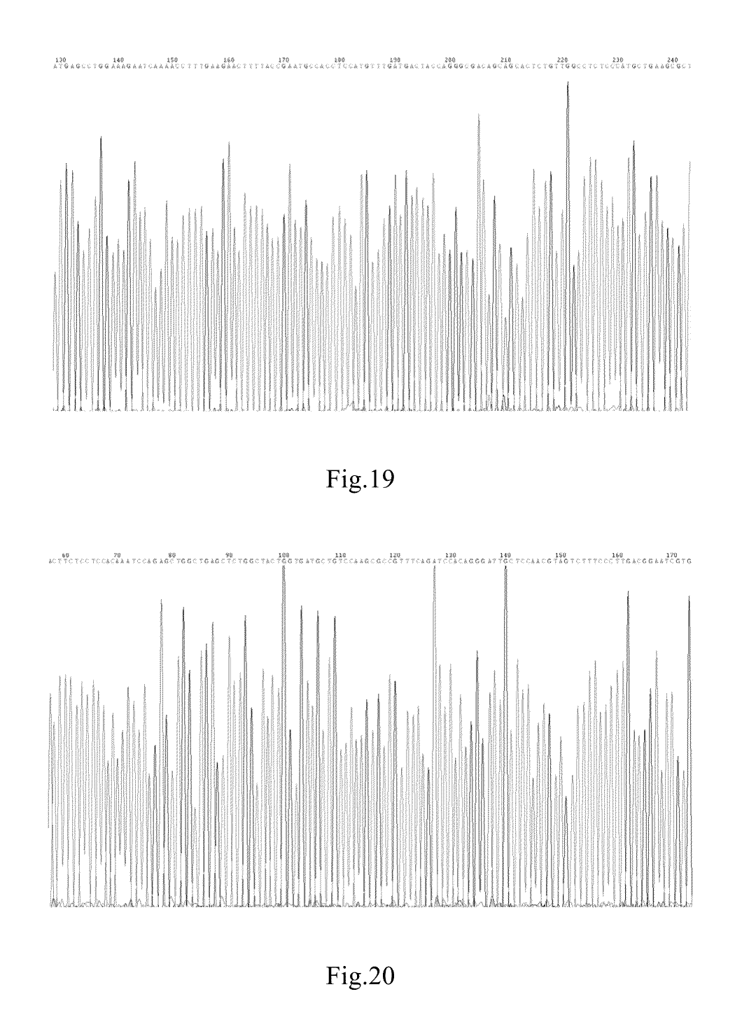

1.6 Sequence the strains having positive result, and use the strains with correct sequence as the positive standard (FIG. 19).

### Example 15

### Preparation of VEGFR2 Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 21):

TABLE 21
PCR primers

| name | sequence | |
|---|---|---|
| VEGFR2-F-1 | TGGAGCAATCCCTGTGGATCT | (SEQ ID NO: 93) |
| VEGFR2-F-2 | GGAGCAATCCCTGTGGATCT | (SEQ ID NO: 94) |
| VEGFR2-R-1 | CTCCTCCACAAATCCAGAGCT | (SEQ ID NO: 95) |
| VEGFR2-R-2 | CCTCCACAAATCCAGAGCT | (SEQ ID NO: 96) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 20).

### Example 16

### Preparation of HER2 Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 22):

TABLE 22
PCR primers

| name | sequence | |
|---|---|---|
| HER2-F-1 | GAAGGACATCTTCCACAAG | ( SEQ ID NO: 97 ) |
| | AACAA | |
| HER2-F-2 | TGCTGTCCTGTTCACCACTC | ( SEQ ID NO: 98 ) |
| HER2-R-1 | GAGCCCTTACACATCGGAGA | ( SEQ ID NO: 99 ) |
| | AC | |
| HER2-R-2 | GCTTTGCATGTGGTCTTGAA | ( SEQ ID NO: 100 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 21).

### Example 17

### Preparation of EGFR Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 23):

TABLE 23
PCR primers

| name | sequence | |
|---|---|---|
| EGFR-F-1 | CCCTCCTGAGCTCTCTGAGT | ( SEQ ID NO: 101 ) |
| EGFR-F-2 | TGCAACCAGCAACAAT | ( SEQ ID NO: 102 ) |
| EGFR-R-1 | CTTGATGGGACAGCTTTGCA | ( SEQ ID NO: 103 ) |
| EGFR-R-2 | GAAGCTGTCTTCCTTGAT | ( SEQ ID NO: 104 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

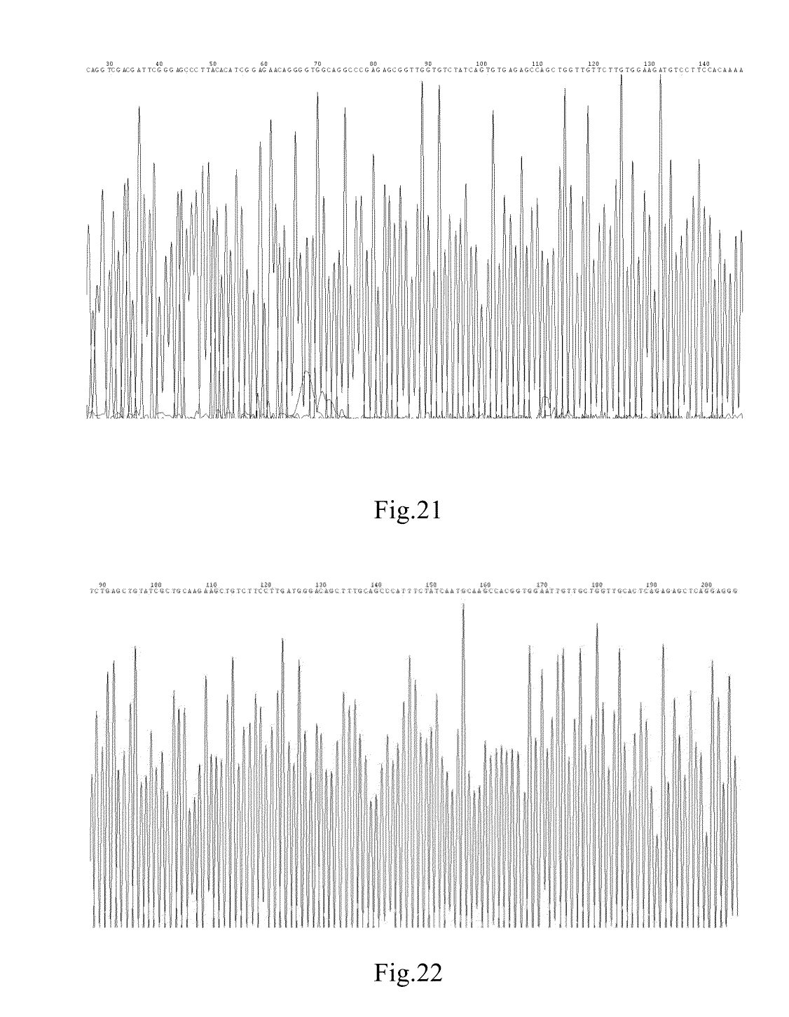

1.6 Sequence the strains having positive result, and use the strains with correct sequence as the positive standard (FIG. 22).

### Example 18

### Preparation of VEGF Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 24):

TABLE 24
PCR primers

| name | sequence | |
|---|---|---|
| VEGF-F-1 | GCCTTGCCTTGCTGCTCTA | ( SEQ ID NO: 105 ) |
| VEGF-F-2 | CTGCTGTCTTGGGTGCATTG | ( SEQ ID NO: 106 ) |
| VEGF-R-1 | TGATTCTGCCCTCCTCCTTC | ( SEQ ID NO: 107 ) |
| | T | |
| VEGF-R-2 | GATTCTGCCCTCCTCCTTCT | ( SEQ ID NO: 108 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

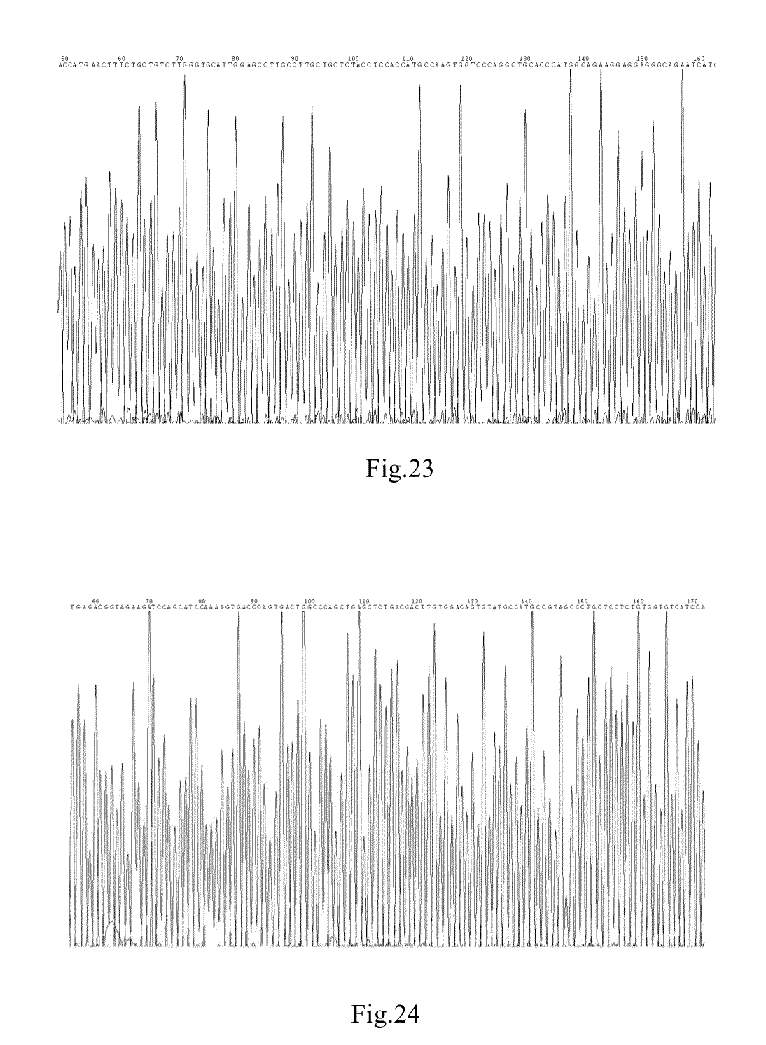

1.6 Sequence the strains having positive result, and use the strains with correct sequence as the positive standard (FIG. 23).

### Example 19

### Preparation of PPN Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 25):

TABLE 25
PCR primers

| name | sequence | |
|---|---|---|
| PPN-F-1 | TCCTCAGTGCCTGTGTCTTG | ( SEQ ID NO: 109 ) |
| PPN-F-2 | GATGTCGATGTGGATGA | ( SEQ ID NO: 110 ) |
| PPN-R-1 | GCATCCAAAAGTGACCCAGT | ( SEQ ID NO: 111 ) |
| PPN-R-2 | CACTTGTGGACAGTGTATG | ( SEQ ID NO: 112 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 24).

### Example 20

### Preparation of CCNB2 Positive Standard (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 26):

TABLE 26
PCR primers

| name | sequence | |
|---|---|---|
| CCNB2-F-1 | GGACATTGATAACGAAGATTG | ( SEQ ID NO: 113 ) |
| CCNB2-F-2 | CACAGGATACACAGAGAATG | ( SEQ ID NO: 114 ) |
| CCNB2-R-1 | GCTGCCTGAGATACTGAT | ( SEQ ID NO: 115 ) |
| CCNB2-R-2 | CTTGATGGCGATGAATTTAG | ( SEQ ID NO: 116 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

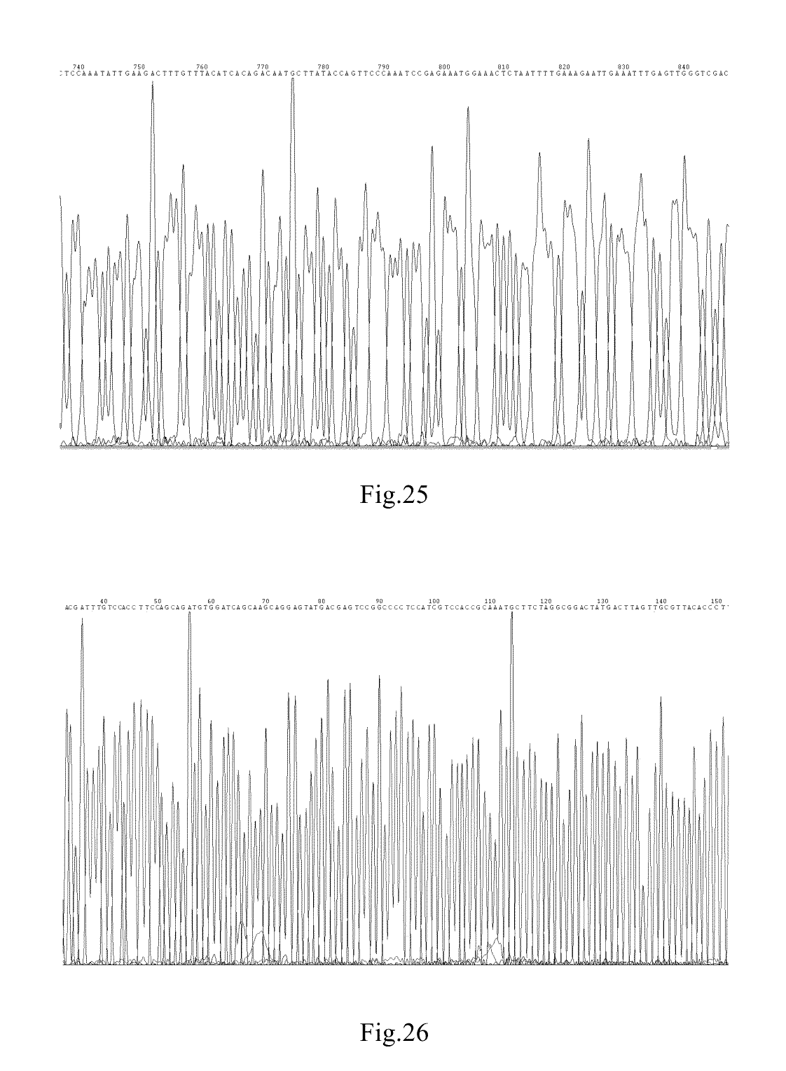

1.6 Sequence the strains having positive result, and use the strains with correct sequence as the positive standard (FIG. 25).

### Example 21

### Preparation of the Positive Standard for Detecting ACTB Expression (FIG. 1, FIG. 2)

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 27):

TABLE 27
PCR primers

| name | sequence | |
|---|---|---|
| ACTB-F-1 | CAGATGTGGATCAGCAAGCA | ( SEQ ID NO: 117 ) |
| ACTB-F-2 | AGAAAATCTGGCACCACACC | ( SEQ ID NO: 118 ) |
| ACTB-R-1 | TCATAGTCCGCCTAGAAGCA | ( SEQ ID NO: 119 ) |
| | TT | |
| ACTB-R-2 | AGAGGCGTACAGGGATAGCA | ( SEQ ID NO: 120 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 26).

### Example 22

### Preparation of the Positive Standard for Detecting 18S rRNA Expression

1. Preparation of the Vector

TA cloning vector pMD18-T was purchased from TAKARA Inc.

2. Preparation of the Insert

The insert is prepared using PCR. The template of PCR is the cDNA prepared in Example 1. The reaction system and amplification condition are shown in the following tables (Table 2, Table 3 and Table 28):

TABLE 28
PCR primers

| name | sequence | |
|---|---|---|
| 18S-F-1 | ACATCCAAGGAAGGCAGCAG | ( SEQ ID NO: 121 ) |
| 18S-F-2 | ACATCCAAGGAAGGCAGCAG | ( SEQ ID NO: 122 ) |
| 18S-R-1 | TTCGTCACTACCTCCCCGG | ( SEQ ID NO: 123 ) |
| 18S-R-2 | TTCGTCACTACCTCCCCGG | ( SEQ ID NO: 124 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

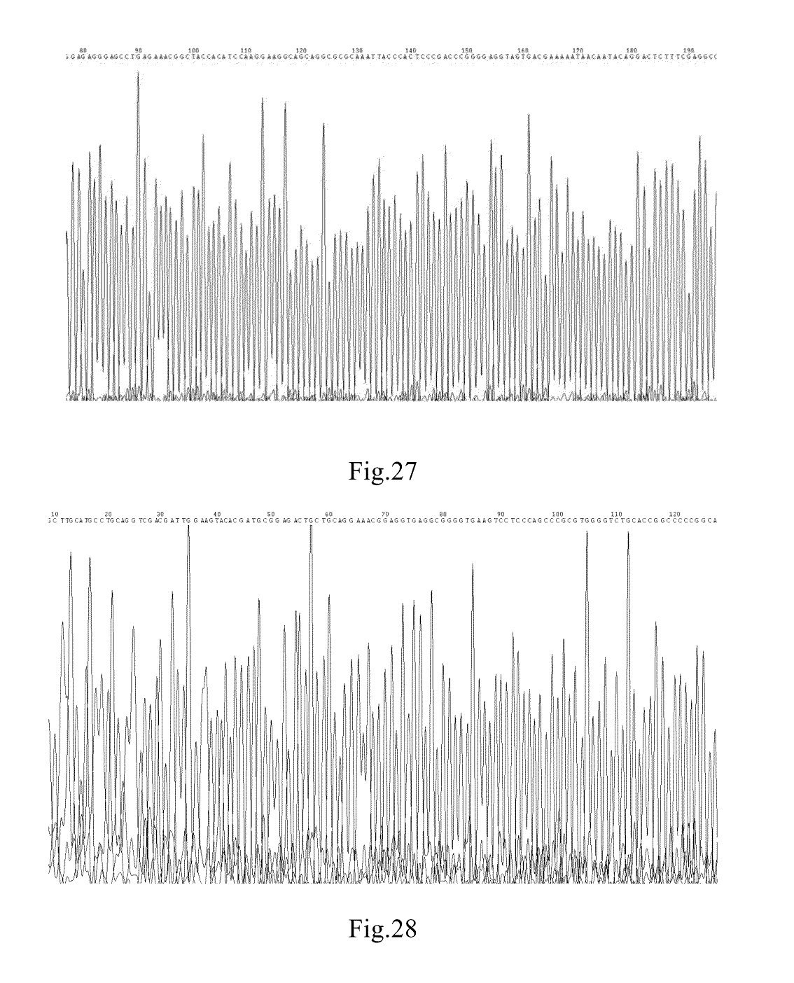

1.6 Sequence the strains having positive result, and use the strains with correct sequence as the positive standard (FIG. 27).

### Example 23

### Preparation of the Positive Standard for Detecting HER2 Gene Amplification

1. Preparation of the Vector

TA cloning vector pMD18-T was purchased from TAKARA Inc.

2. Preparation of the Insert

The insert is prepared using PCR. The template of PCR is the sample genome DNA extracted in Example 1. The reaction system and amplification condition are shown in the following tables (Table 2, Table 3 and Table 29):

TABLE 29
PCR primers

| name | sequence | |
|---|---|---|
| O-HER2-F-1 | GAAAGAGACGGAGCTGAG | ( SEQ ID NO: 125 ) |
| | GAA | |
| O-HER2-F-2 | CAGACCATTTGGGTTCAA | ( SEQ ID NO: 126 ) |
| | ATCC | |
| O-HER2-R-1 | GGCCCTGACCTTGTAGAC | ( SEQ ID NO: 127 ) |
| | TGT | |
| O-HER2-R-2 | GAGACCAAAGCAGAGAGT | ( SEQ ID NO: 128 ) |
| | TCT | |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

1.6 Sequence the strains having positive result, and use the strains with correct sequence as the positive standard (FIG. 28).

### Example 24

### Preparation of the Positive Standard for Detecting ACTB Gene Amplification

1. Preparation of the Vector

TA cloning vector pMD18-T was purchased from TAKARA Inc.

2. Preparation of the Insert

The insert is prepared using PCR. The template of PCR is the sample genome DNA extracted in Example 1. The reaction system and amplification condition are shown in the following tables (Table 2, Table 3 and Table 30):

TABLE 30
PCR primers

| name | sequence | |
|---|---|---|
| ACTB-F-1 | CAGGATGCAGAAGGAGAT | ( SEQ ID NO: 129 ) |
| | CACT | |
| ACTB-F-2 | CATCCTCACCCTGAAGTA | ( SEQ ID NO: 130 ) |
| ACTB-R-1 | CAGCTCAGGCAGGAAAGA | ( SEQ ID NO: 131 ) |
| | CA | |
| ACTB-R-2 | ACACGCAGCTCATTGTAG | ( SEQ ID NO: 132 ) |

1.3 After recovering the target fragment using QIAgen Gel Recover Kit, insert said fragment into pMD18-T (purchased from TAKARA Inc.) by TA colonizing.

1.4 Amplify the constructed plasmid in E. coli DH5α strain, and harvest by extraction and purification (the methods are showed in Molecular Cloning, A Laboratory Manual, 3^{rd} ed. pages 96-99 and 103).

1.5 Identify the plasmid by double enzyme digestion of BamHI and HindIII.

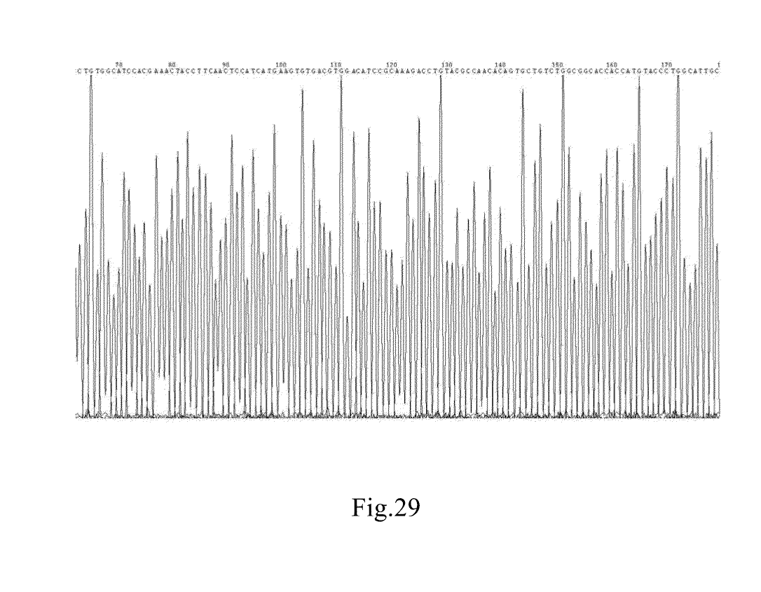

1.6 Sequence the strains having positive result, and use the strains with correct sequence as the positive standard (FIG. 29).

### Example 25

### Use of Positive Plasmid Standard in Mutant Test

1. Prepare plasmid standard with different concentration

Double dilute the plasmid standard to 5E-1 ng/μl, 2.5E-1 ng/μl, 1.25E-1 ng/μl, 6.25E-2 ng/μl, 3.125E-2 ng/μl, as the template for fluorescent quantitative PCR.

2. Prepare DNA of the sample using the method of Example 1.

3. The reaction system and amplification condition for fluorescent quantitative PCR (Table 31, Table 32)

For detecting different genes, we should add corresponding primers (Table 4, Table 6, Table 8 and Table 10) and probes (Table 33-Table 36) into the reaction system, wherein probes are labeled with fluorescence emitting groups which are selected from FAM, TET, HEX, or ROX, and fluorescence quenching groups which are selected from BHQ or TAMARA.

TABLE 31
fluorescent quantitative PCR reaction system (20 μl/tube)

| reagents | amount (μl/tube) |
|---|---|
| double-distilled water | 9.9 |
| 10x buffer (free of Mg²⁺) | 2 |
| MgCl₂ (25 mM) | 3 |
| dNTP (10 mM) | 0.5 |
| upstream primer (25 μM) | 0.5 |
| downstream primer (25 μM) | 0.5 |
| fluorescent probe (25 μM) | 0.2 |
| Taq enzyme | 0.4 |
| DNA template | 3 |
| total volume | 20 |

TABLE 32
amplification condition

| step | cycles | temperature and time |
|---|---|---|
| step 1 | 1 | 95° C., 1-5 minutes |
| step 2 | 30-45 | 95° C., 10-15 seconds; 55-65° C. (collect fluorescence), |
| | | 30-60 seconds |

TABLE 33
Probes for fluorescent quantitative
PCR detection of EGFR mutant

| name | sequence | |
|---|---|---|
| E18W-1 | GGCTCCGGTGCGTTCGGC | ( SEQ ID NO: 133 ) |
| E18W-2 | CGGAGCCCAGCACTTTGATCT | ( SEQ ID NO: 134) |
| E18M-1 | TGCTGAGCTCCGGTGCGTT | ( SEQ ID NO: 135 ) |
| E18M-2 | CGGAGCTCAGCACTTTGATCTT | ( SEQ ID NO: 136 ) |
| E19W-1 | TCAAGGAATTAAGAGAAGCAAC | ( SEQ ID NO: 137 ) |
| | ATC | |
| E19W-2 | CGGAGATGTTGCTTCTCTTAAT | ( SEQ ID NO: 138 ) |
| | TCCT | |
| E19M1-1 | CCCGTCGCTATCAAAACATCT | ( SEQ ID NO: 139 ) |
| E19M1-2 | AGATGTTTTGATAGCGACGGG | ( SEQ ID NO: 140 ) |
| E19M2-1 | CCCGTCGCTATCAAGACATCTC | ( SEQ ID NO: 141 ) |
| E19M2-2 | GAGATGTCTTGATAGCGACGGG | ( SEQ ID NO: 142 ) |
| E19M3-1 | AATTAAGAGAAGCAACACTCGAT | ( SEQ ID NO: 143 ) |
| E19M3-1 | ATCGAGTGTTGCTTCTCTTAATT | ( SEQ ID NO: 144 ) |
| E21W-1 | TGGCCAGCCCAAAATCTGTG | ( SEQ ID NO: 145 ) |
| E21W-1 | AAGATCACAGATTTTGGGCTGGC | ( SEQ ID NO: 146 ) |
| E21M-1 | TGGCCCGCCCAAAATCTGT | ( SEQ ID NO: 147 ) |
| E21M-1 | GATCACAGATTTTGGGCGGGC | ( SEQ ID NO: 148 ) |

TABLE 34
Probes for fluorescent quantitative PCR
detection of KRAS mutant

| name | sequence | |
|---|---|---|
| KRAS-W₁ | AGCTGGTGGCGTAGGCAAGA | ( SEQ ID NO: 149 ) |
| KRAS-W₂ | AGCTGGTGGCGTAGGCAAGAGT | ( SEQ ID NO: 150 ) |
| KRAS-1₁ | AGCT GTT GGCGTAGGCAAGA | ( SEQ ID NO: 151 ) |
| KRAS-1₂ | AGCTGTTGGCGTAGGCAAGAGTG | ( SEQ ID NO: 152 ) |
| KRAS-2₁ | AGCT AGT GGCGTAGGCAAGA | ( SEQ ID NO: 153 ) |
| KRAS-2₂ | AGCTAGTGGCGTAGGCAAGAGTG | ( SEQ ID NO: 154 ) |
| KRAS-3₁ | AGCT GAT GGCGTAGGCAAGA | ( SEQ ID NO: 155 ) |
| KRAS-3₂ | AGCTGATGGCGTAGGCAAGAGTG | ( SEQ ID NO: 156 ) |
| KRAS-4₁ | AGCT TGT GGCGTAGGCAAGA | ( SEQ ID NO: 157 ) |
| KRAS-4₂ | AGCTTGTGGCGTAGGCAAGAGTG | ( SEQ ID NO: 158 ) |
| KRAS-5₁ | AGCTGGT GAC GTAGGCAAGA | ( SEQ ID NO: 159 ) |
| KRAS-5₂ | AGCTGGTGACGTAGGCAAGAGTG | ( SEQ ID NO: 160 ) |

TABLE 35
Probes for fluorescent quantitative PCR
detection of BCRP mutant

| name | sequence | |
|---|---|---|
| BCRP-W-1 | CCCATGAGGATGTTACCAAG | ( SEQ ID NO: 161 ) |
| | TATT | |
| BCRP-W-2 | TTACCCATGAGGATGTTACC | ( SEQ ID NO: 162 ) |
| | AAGTATT | |
| BCRP-M1-1 | CCCATGGGGATGTTACCAAG | ( SEQ ID NO: 163 ) |
| | TATT | |
| BCRP-M1-2 | TTACCCATGGGGATGTTACC | ( SEQ ID NO: 164 ) |
| | AAGTATT | |
| BCRP-M2-1 | CCCATGACGATGTTACCAAG | ( SEQ ID NO: 165 ) |
| | TATT | |
| BCRP-M2-2 | TTACCCATGACGATGTTACC | ( SEQ ID NO: 166 ) |
| | AAGTATT | |

TABLE 36
Probes for fluorescent quantitative PCR
detection of BRAF mutant

| name | sequence | |
|---|---|---|
| BRAF-W-1 | CCA TCG AGA TTT CAC | ( SEQ ID NO: 167 ) |
| | TGT AG | |
| BRAF-W-2 | CCATCGAGATTTCACTGTA | ( SEQ ID NO: 168 ) |
| | GCTAGACCA | |
| BRAF-M-1 | CCA TCG AGA TTT CTC | ( SEQ ID NO: 169 ) |
| | TGT AG | |
| BRAF-M-2 | CCATCGAGATTTCTCTGTA | ( SEQ ID NO: 170 ) |
| | GCTAGACCA | |

For different kinds of mutations, the detection of them needs different reaction systems. For example, for detecting the mutations in BRAF Codon 600, it needs to prepare two systems, in which all reagents are same except the probes, i.e., all the primers are BRAF-F1 (SEQ ID NO:51) or BRAF-F2 (SEQ ID NO:52) together with BRAF-R1 (SEQ ID NO:53) or BRAF-R2 (SEQ ID NO:54). For detecting BRAF Codon 600 wild-type genes, the system needs to add BRAF-W-1 (SEQ ID NO:167) or BRAF-W-2 (SEQ ID NO:168) probes. For detecting BRAF Codon 600 GTG→GAG mutant, the system needs to add BRAF-M-1 (SEQ ID NO:169) or BRAF-M-2 (SEQ ID NO:170) probes.

4. Draw the standard curve

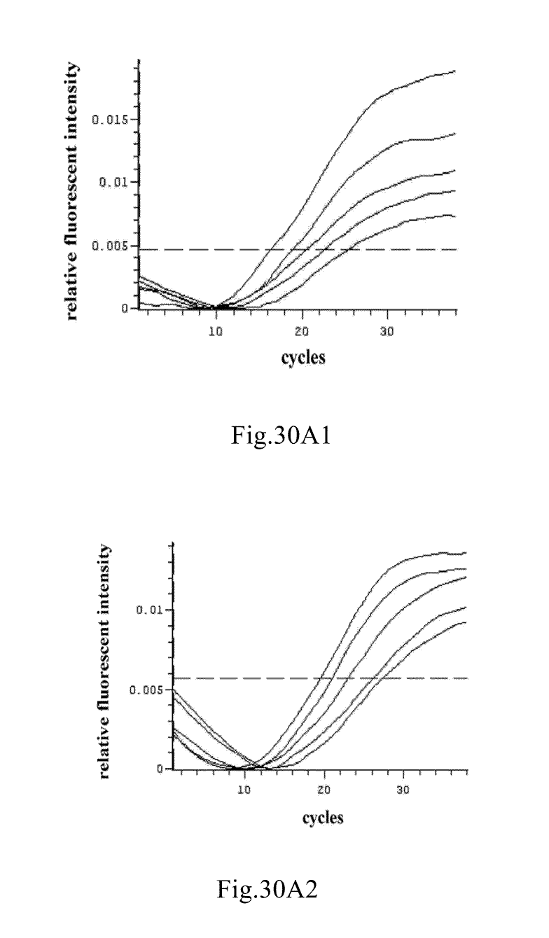

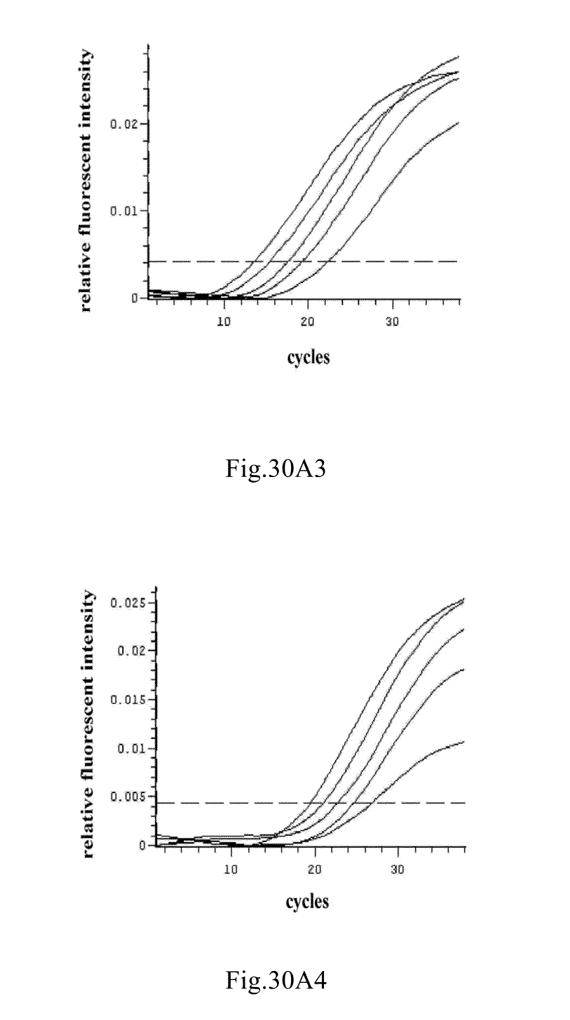

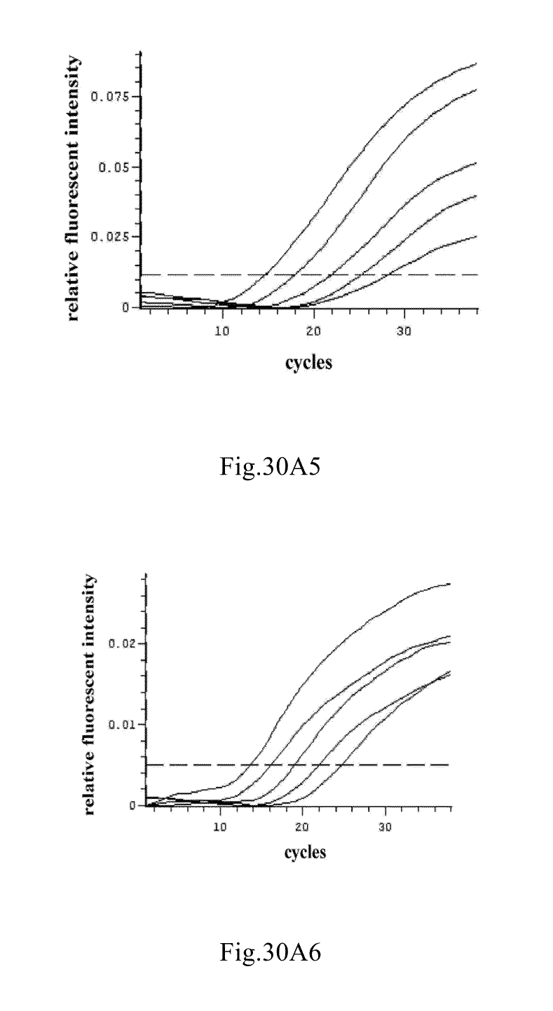

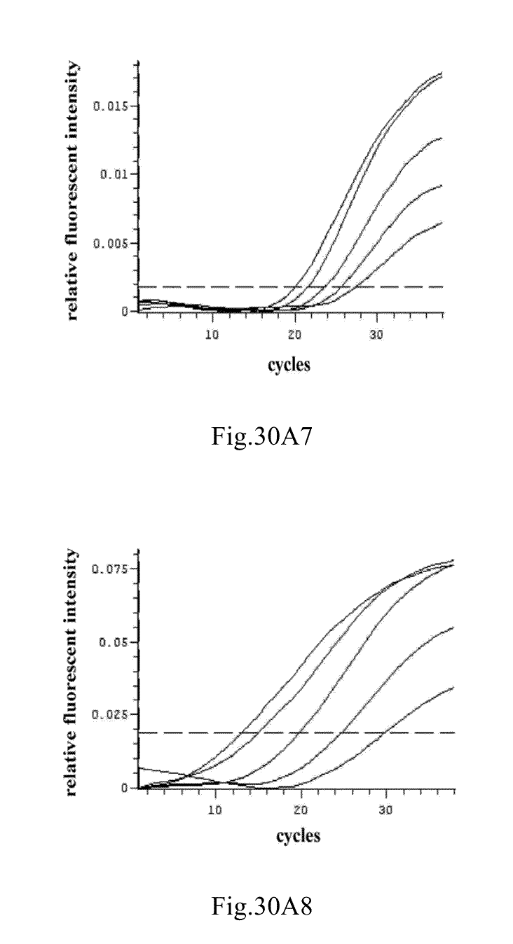

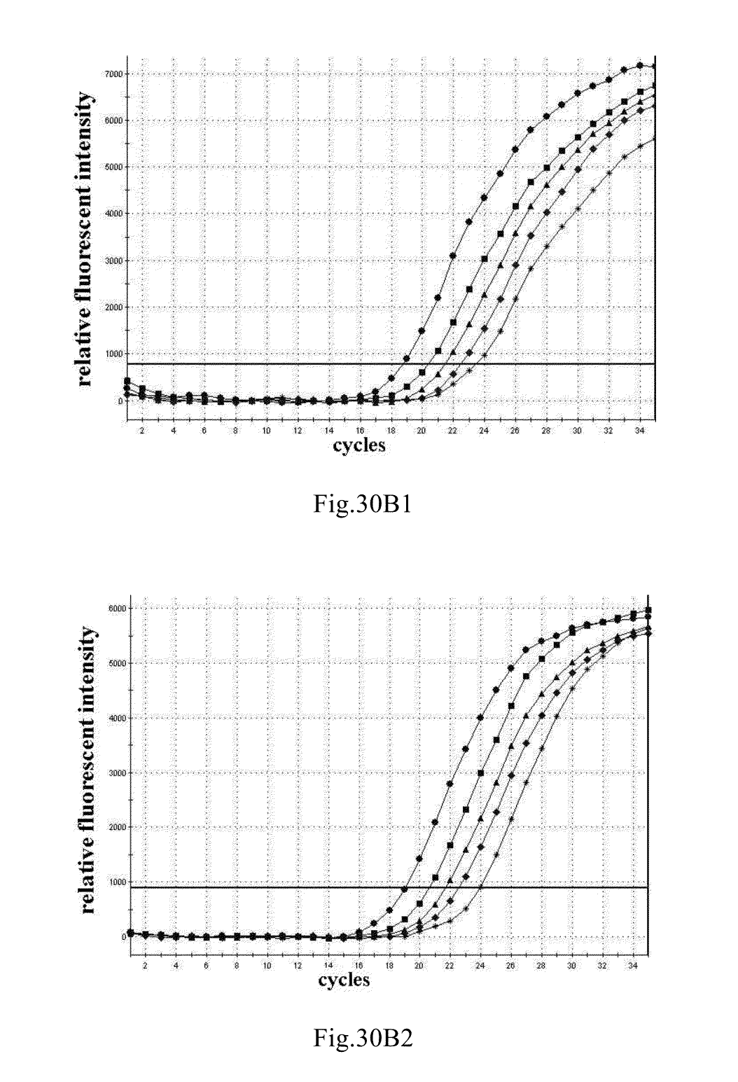

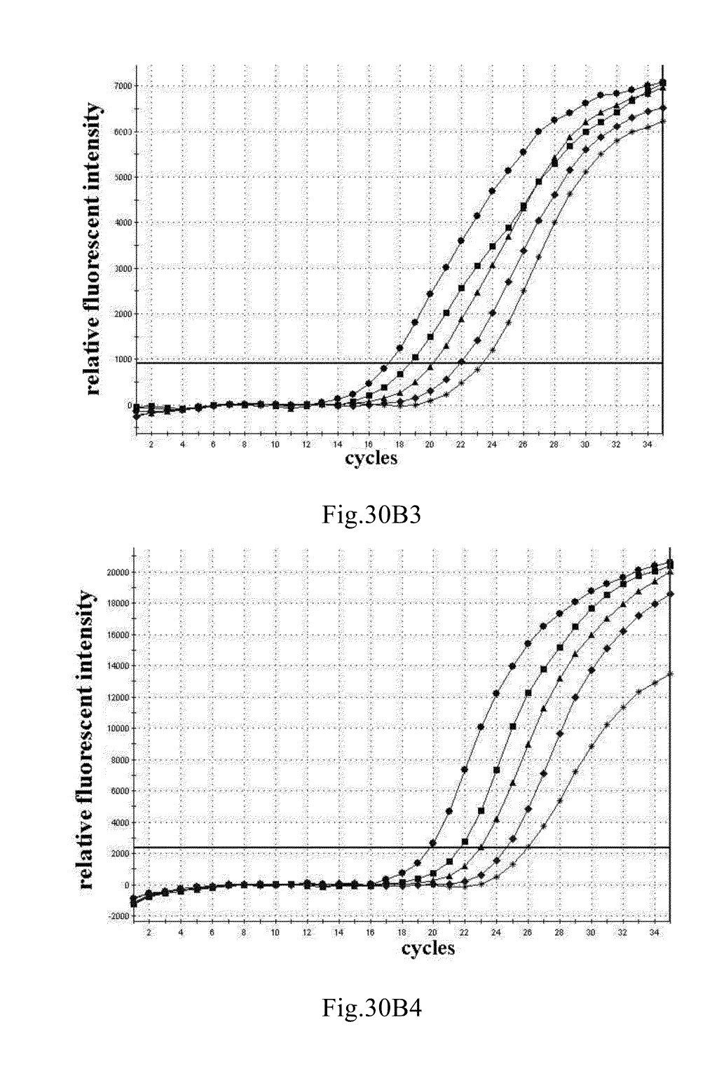

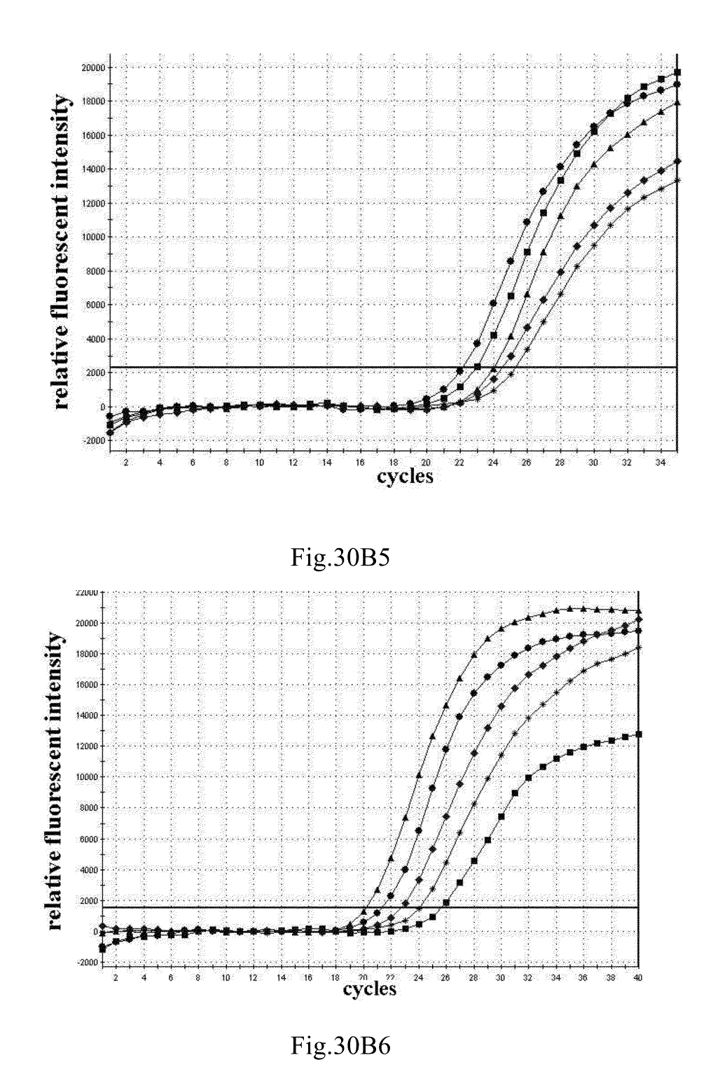

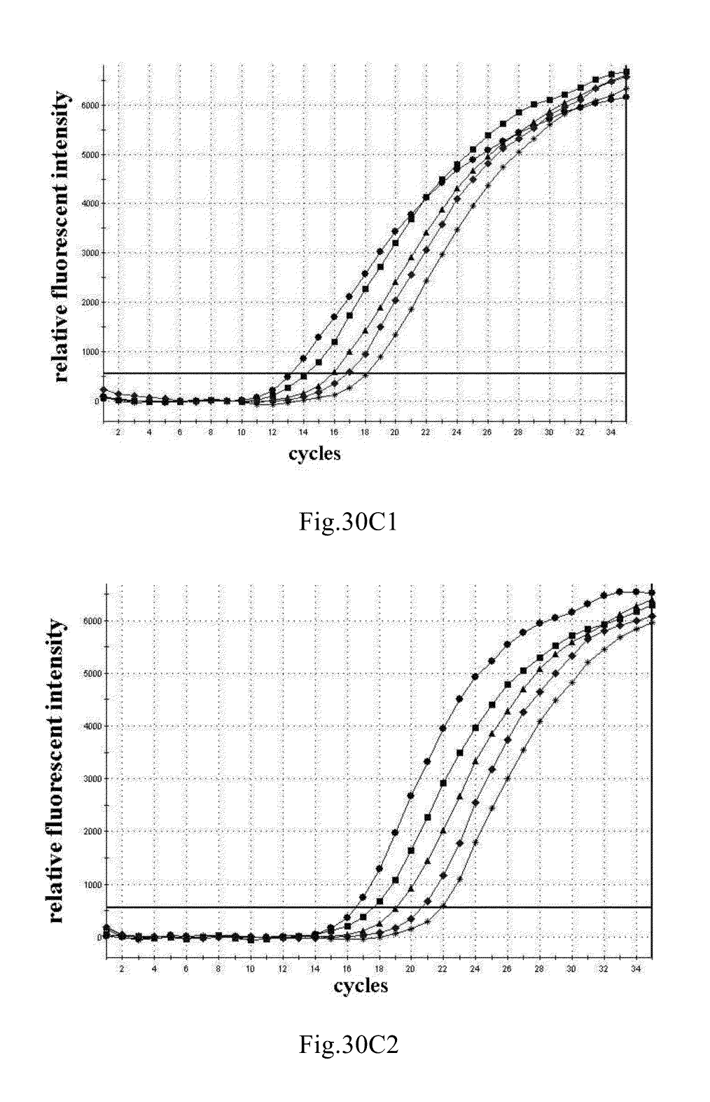

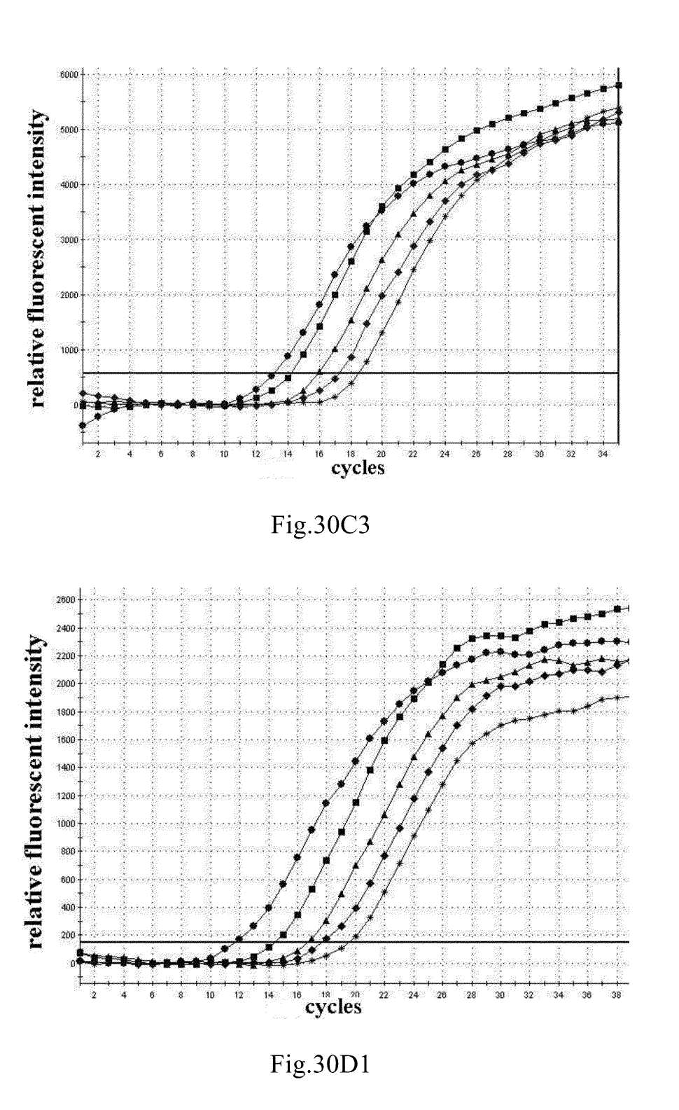

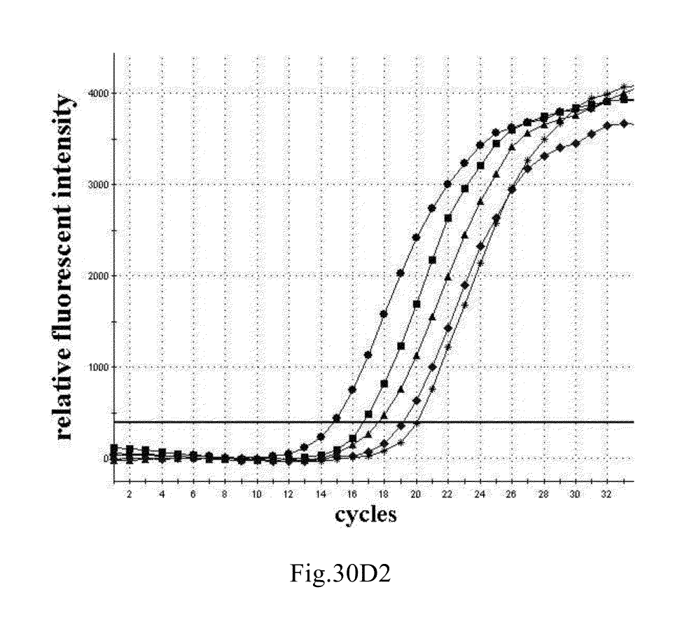

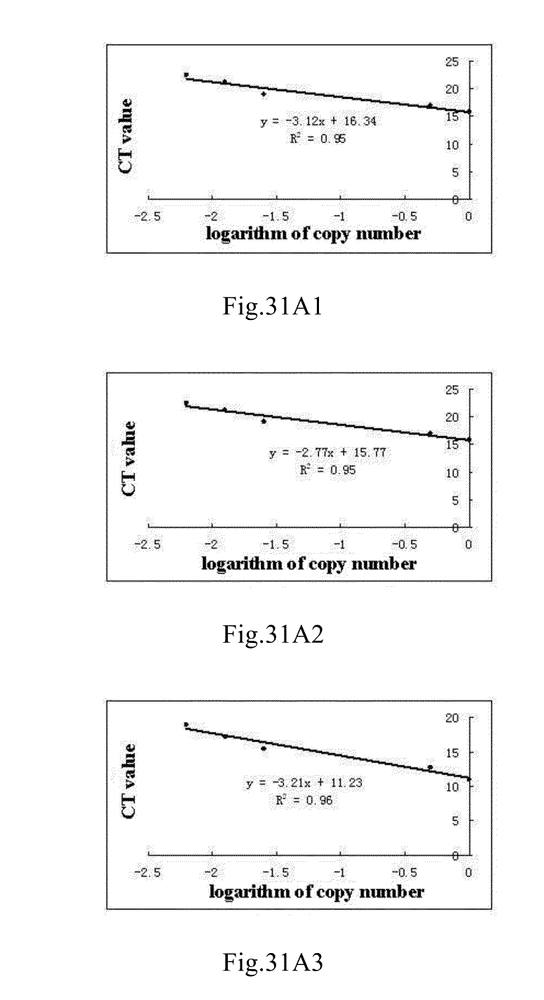

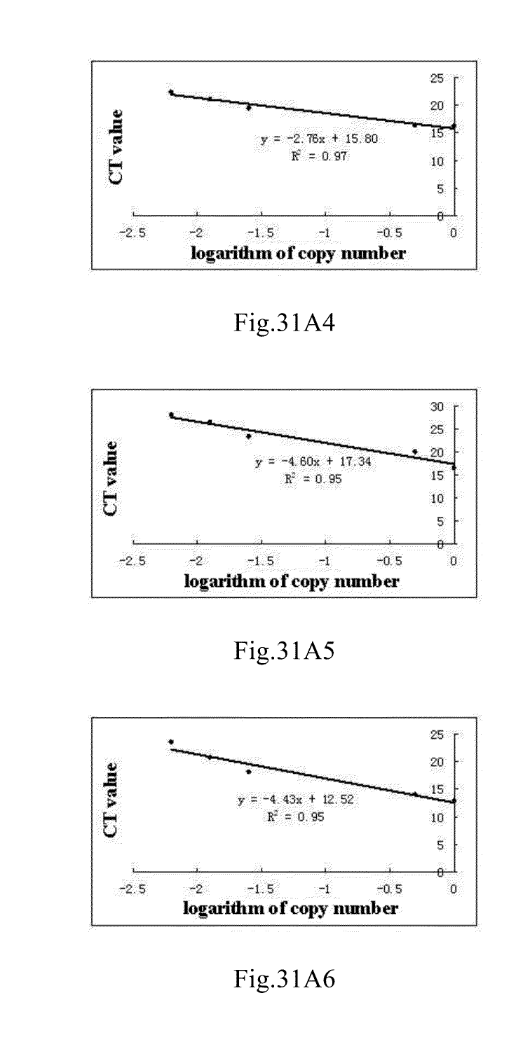

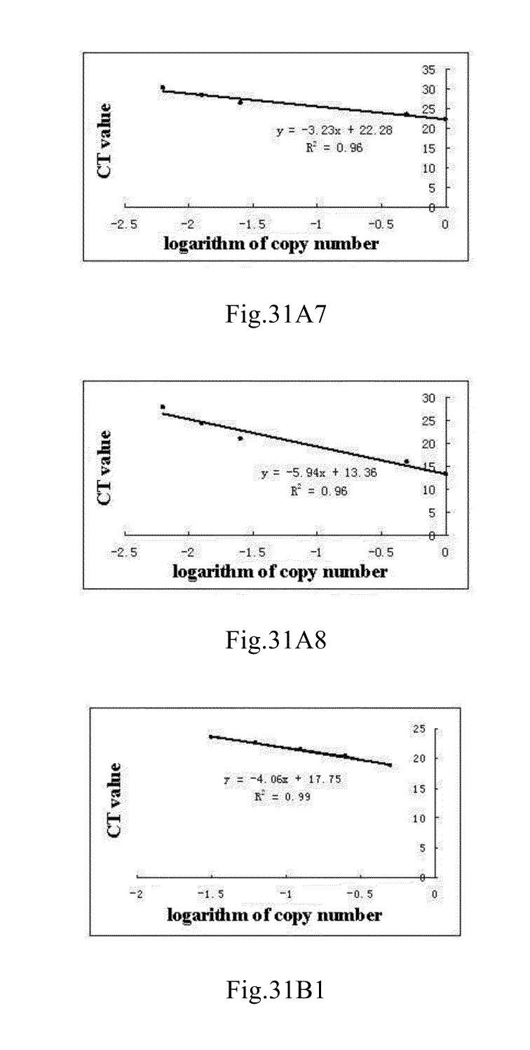

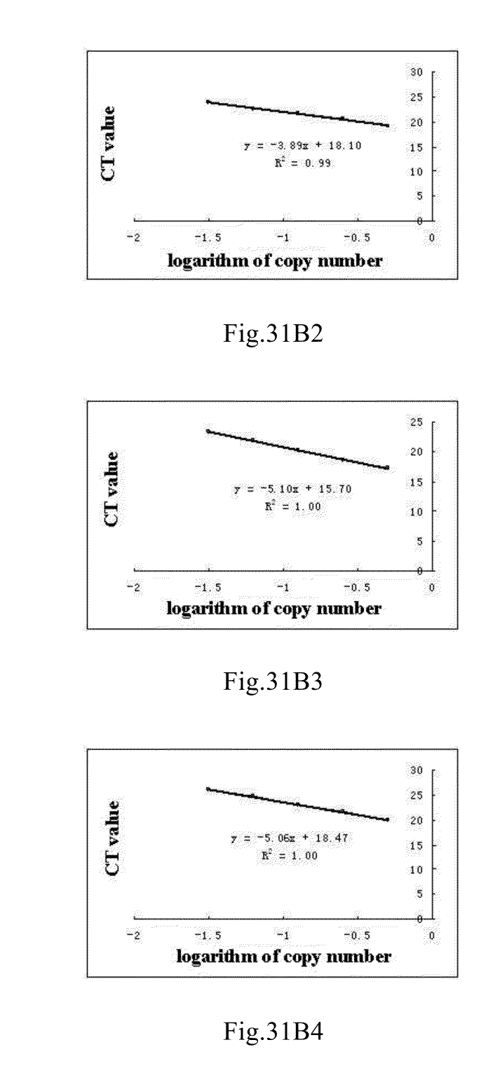

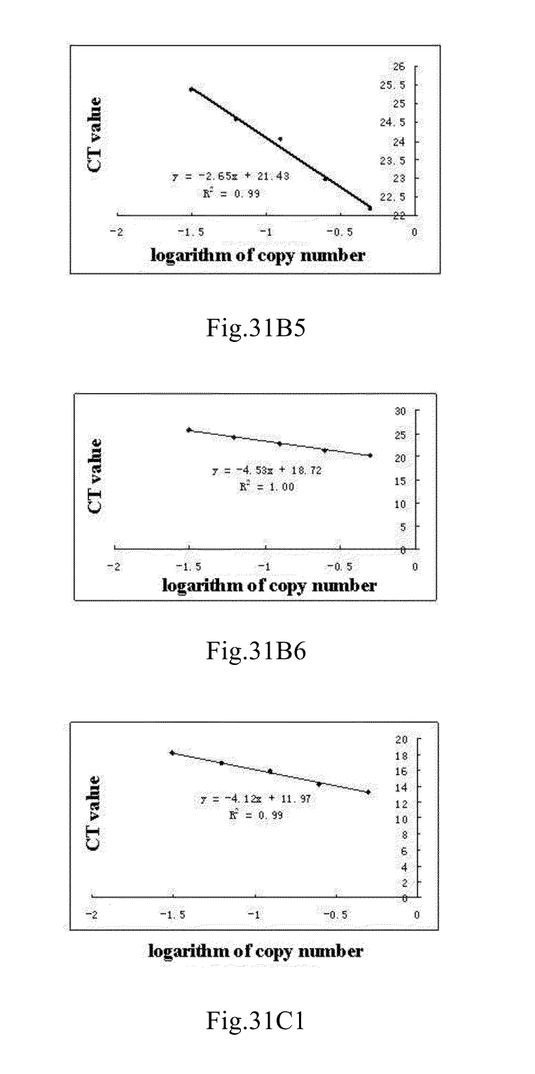

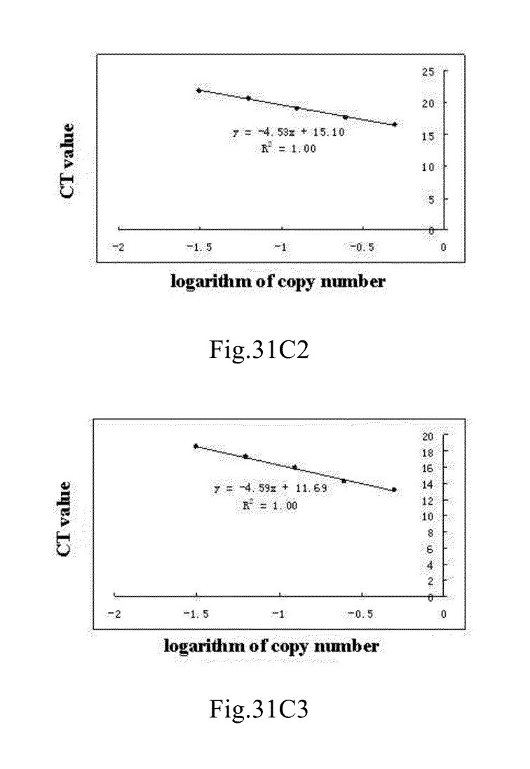

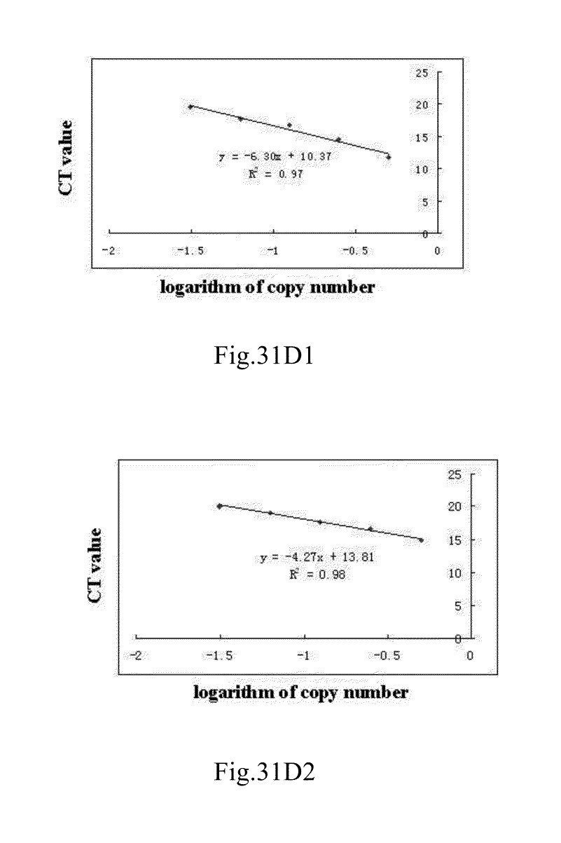

The standard curve is drawn based on the CT values obtained from the standard in Step 3. FIG. 30 shows the amplification curve of plasmid standard, in which the five rising curves represent, from left to right, the amplification curve of the plasmid standard 10 times diluted in turn. The horizontal axis represents cycle number, and the vertical axis represents fluorescent detection value. Accordingly, it is possible to draw the standard curve for calculation (FIG. 31). In FIG. 30, the horizontal axis represents the logarithm of copy number of the template, the vertical axis represents CT value, wherein the copy number of template=mass/(molecular weight)×6.02×10²³. In the present experiment, the plasmid consists of pMD18-T vector and an insert. All of the inserts have a length of about 100 bp, very short compared with that of pMD18-T vector. Therefore, they can be ignored. The copy number of 0.5 ng/μl plasmid is 10¹⁰.

5. Calculate the ratio of gene mutations in a sample using standard curves

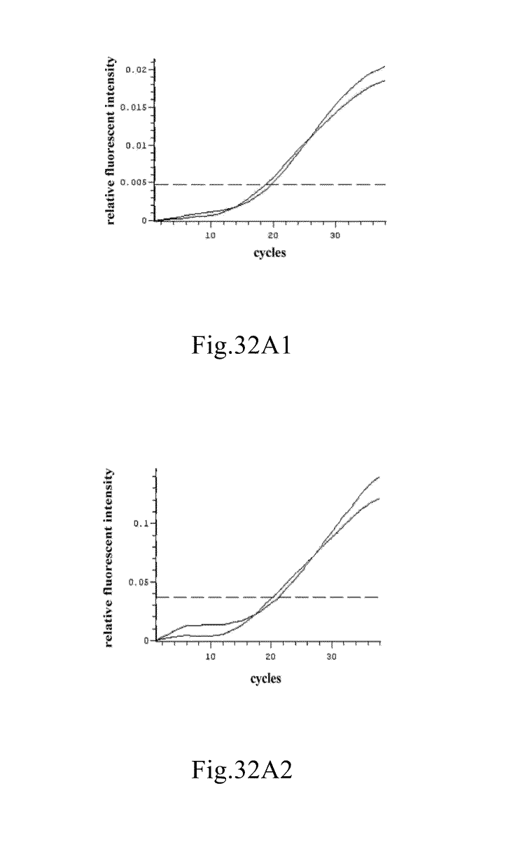

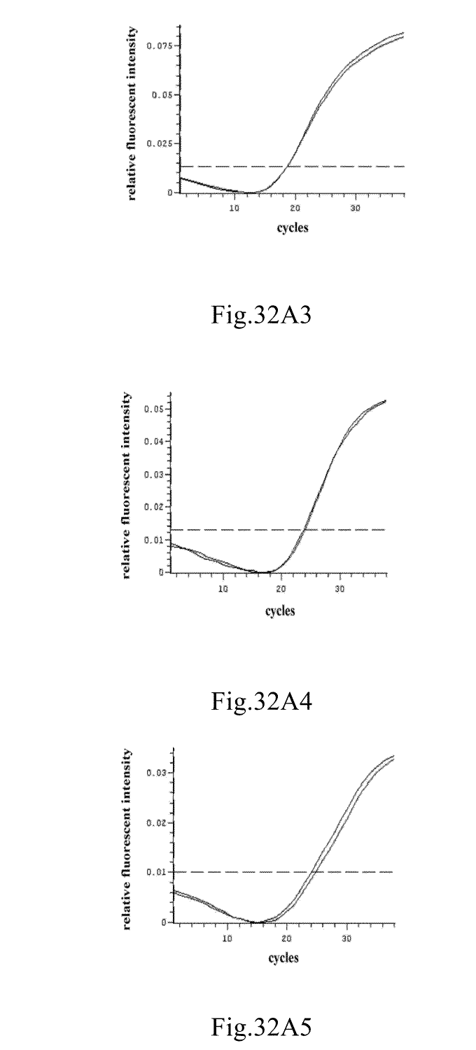

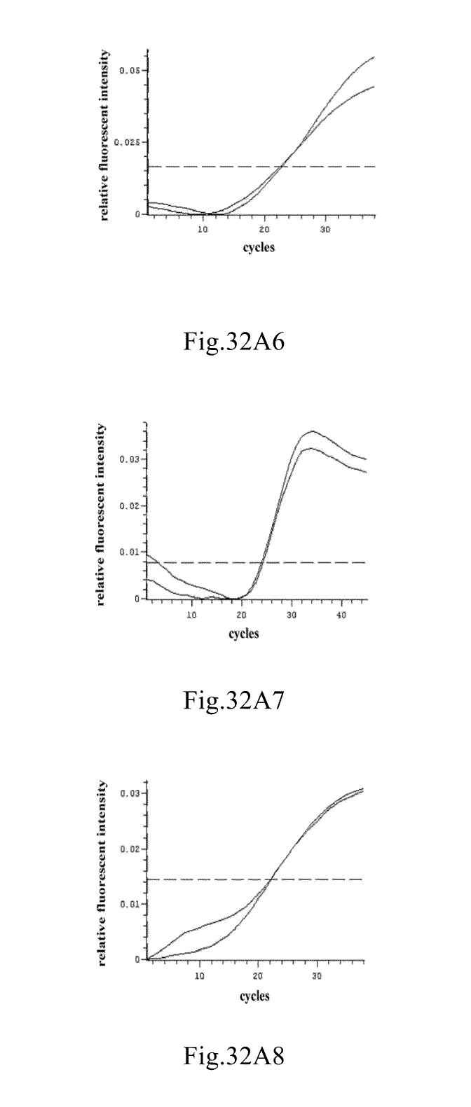

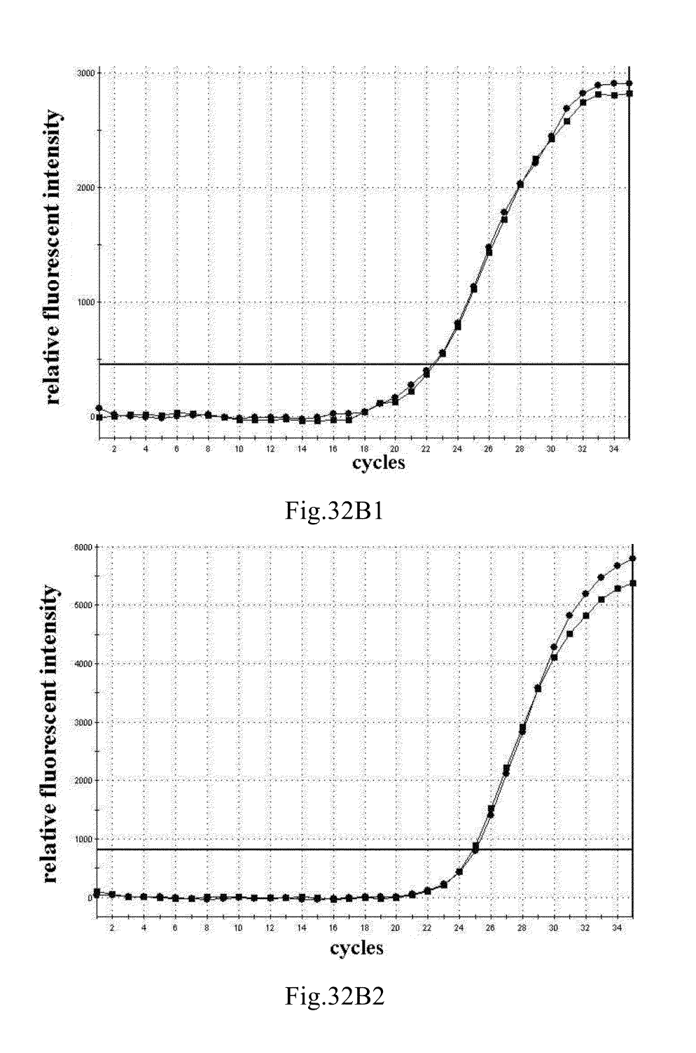

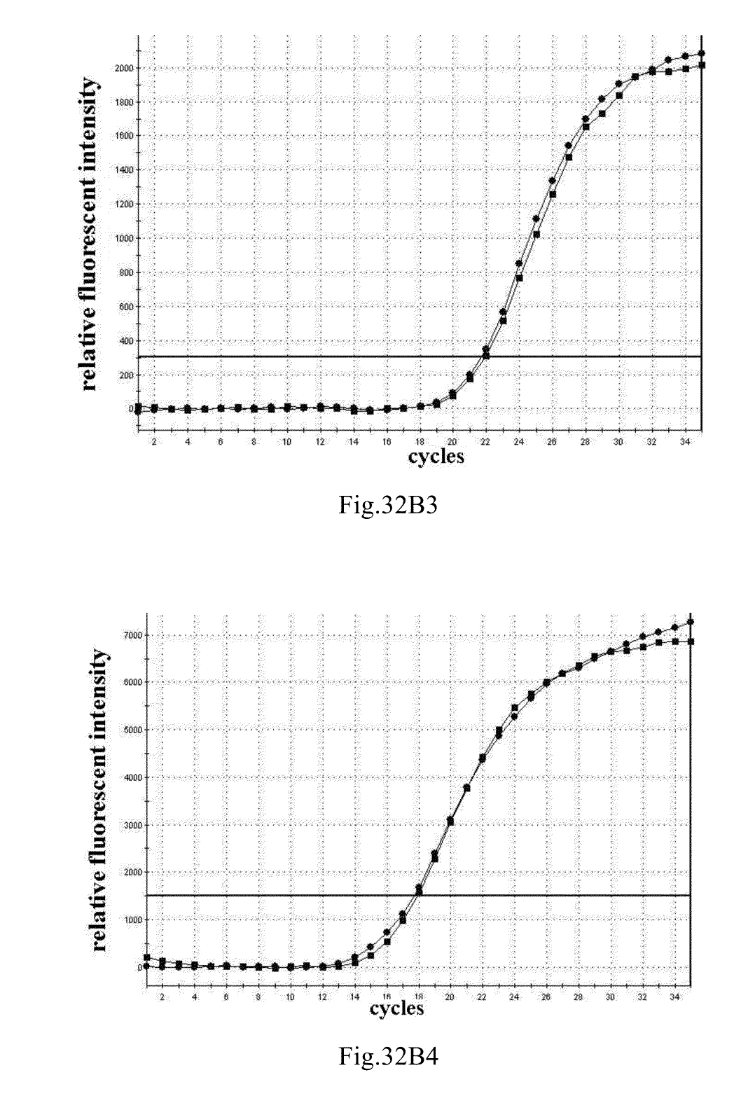

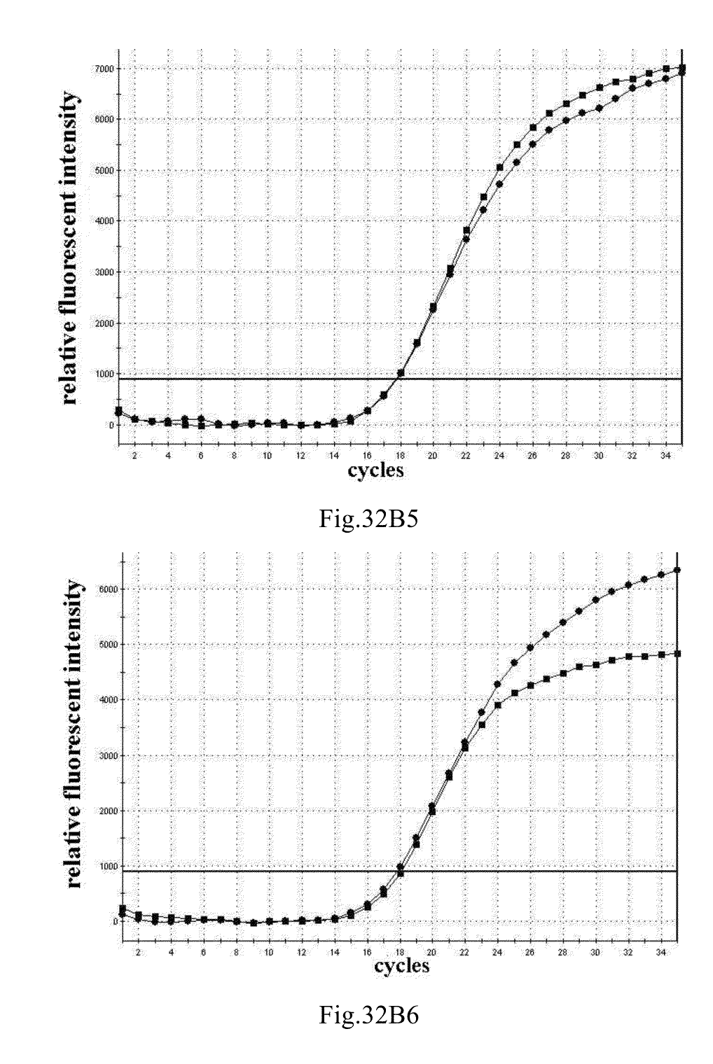

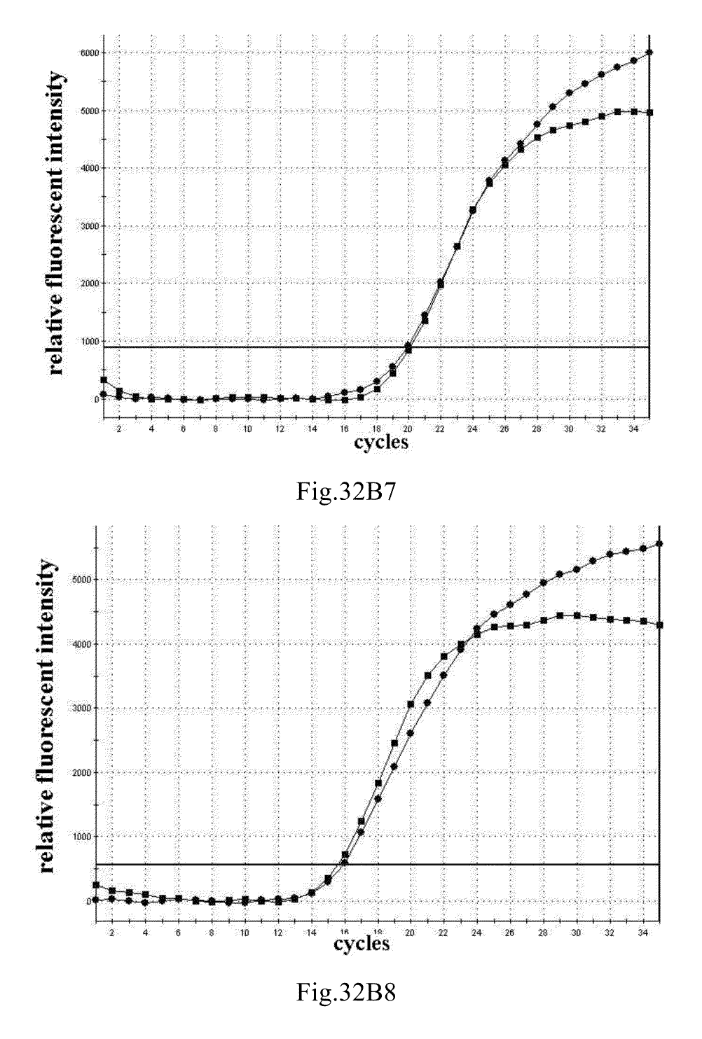

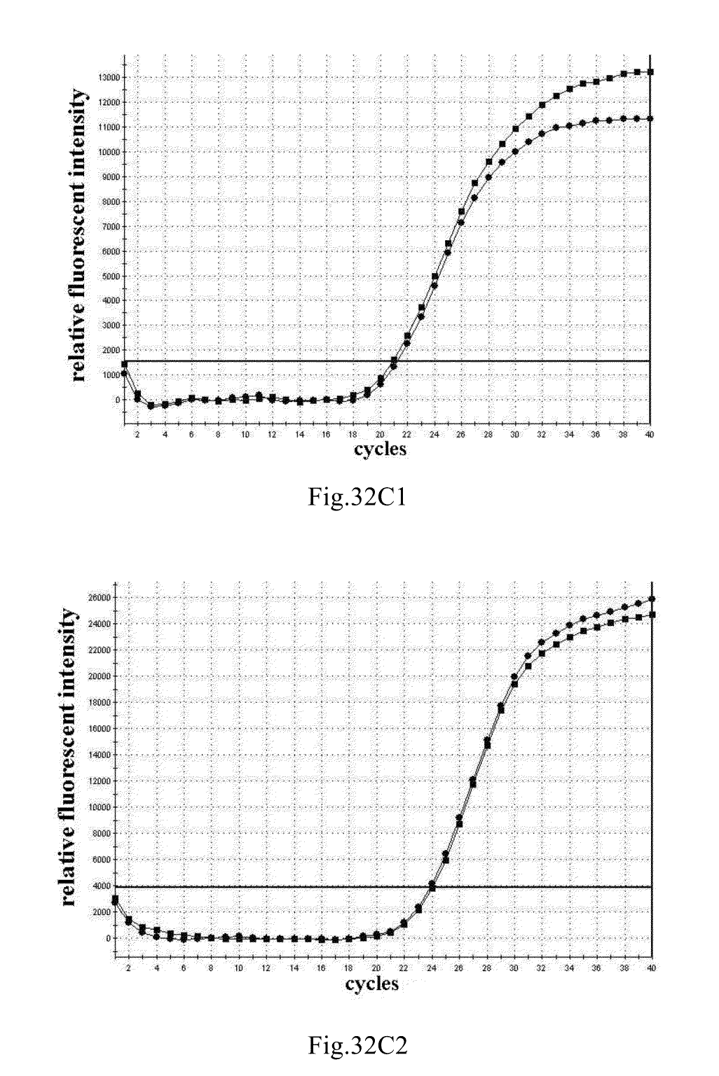

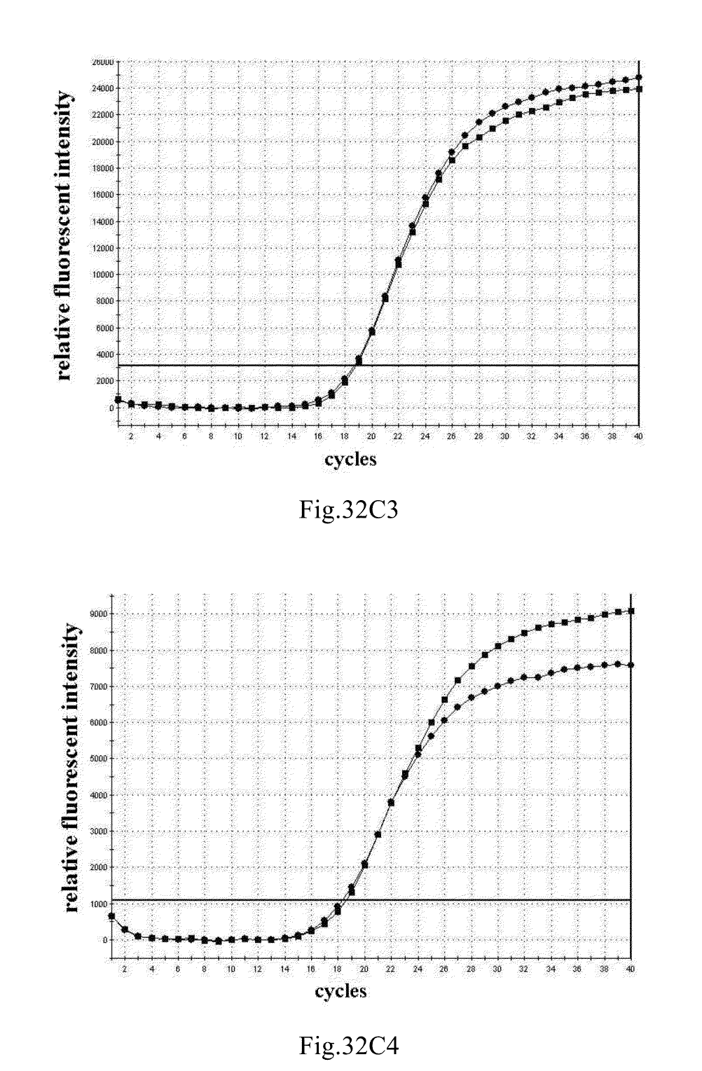

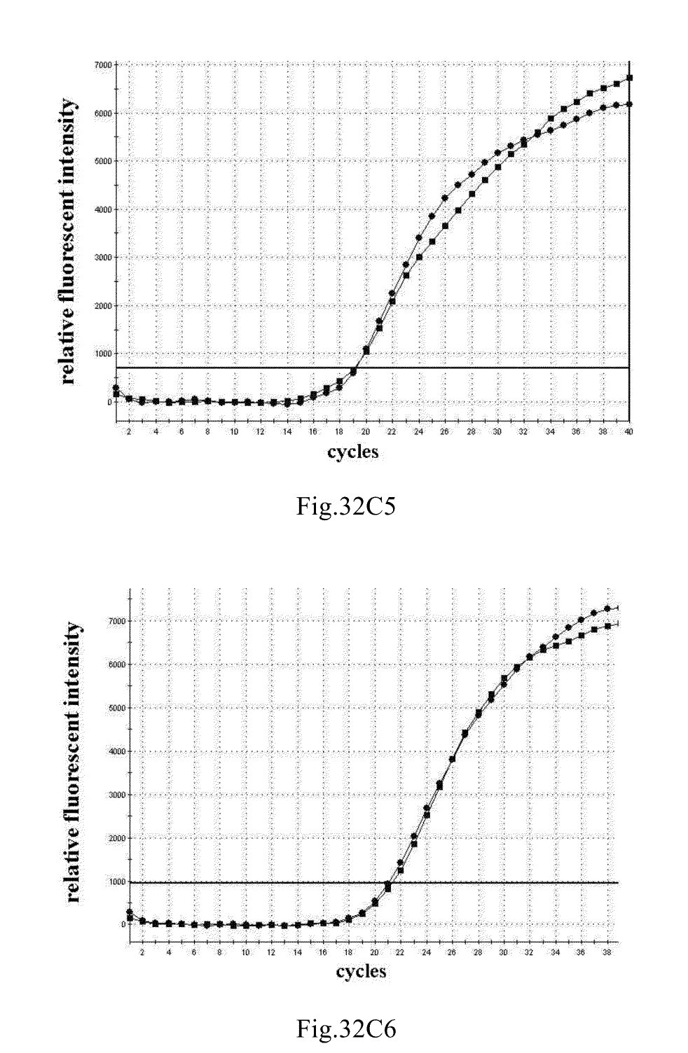

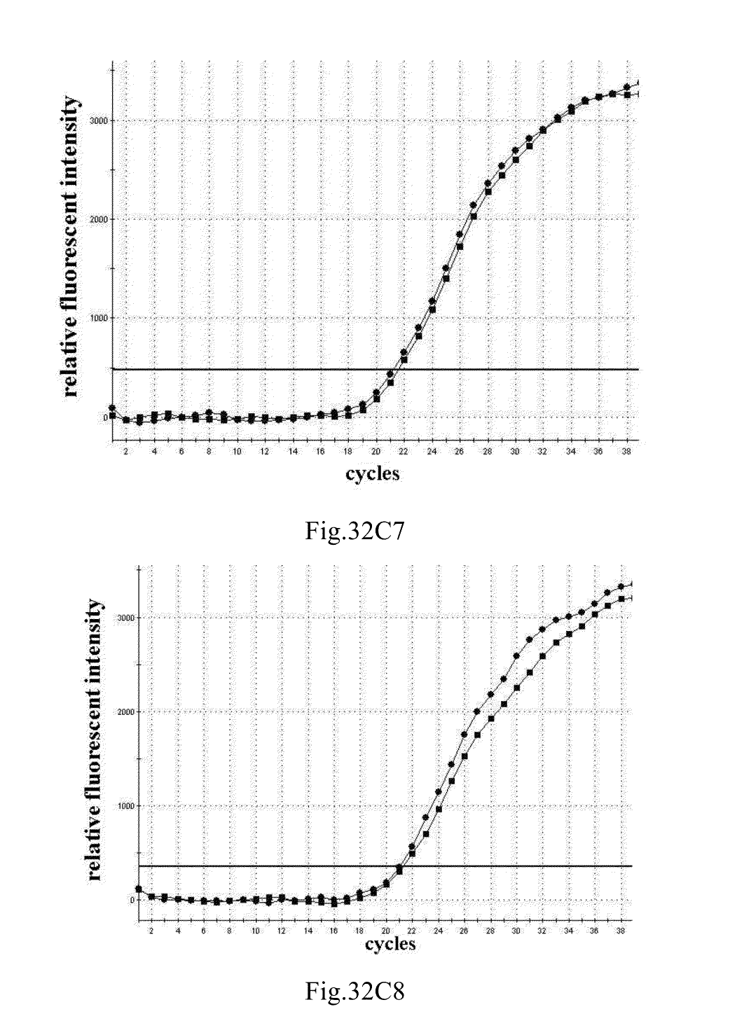

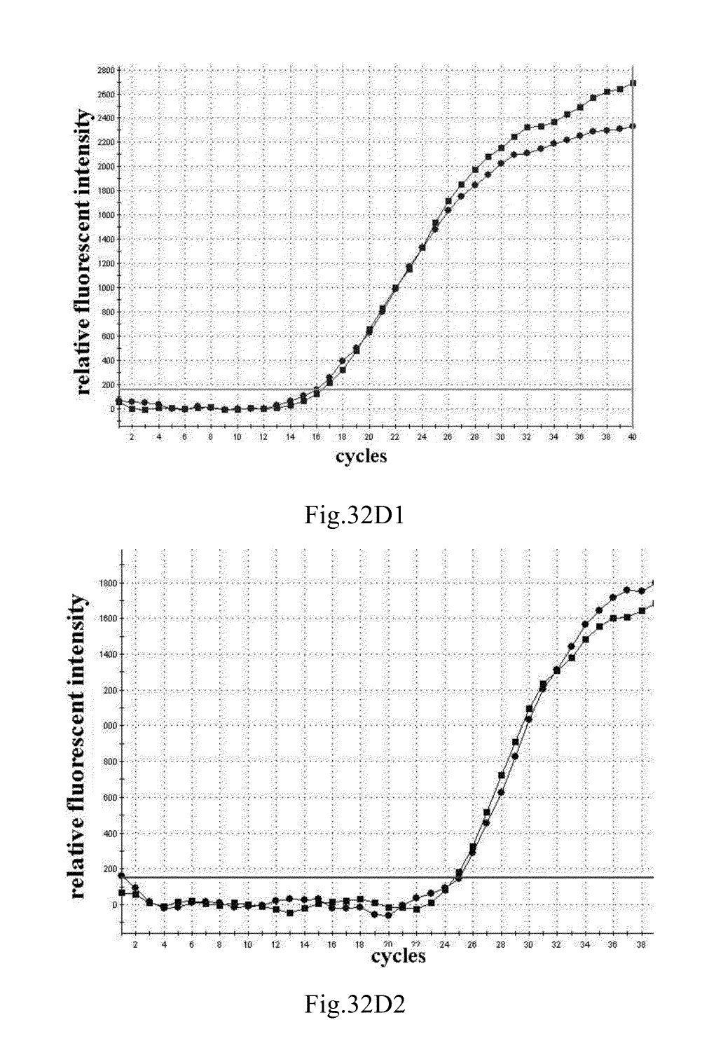

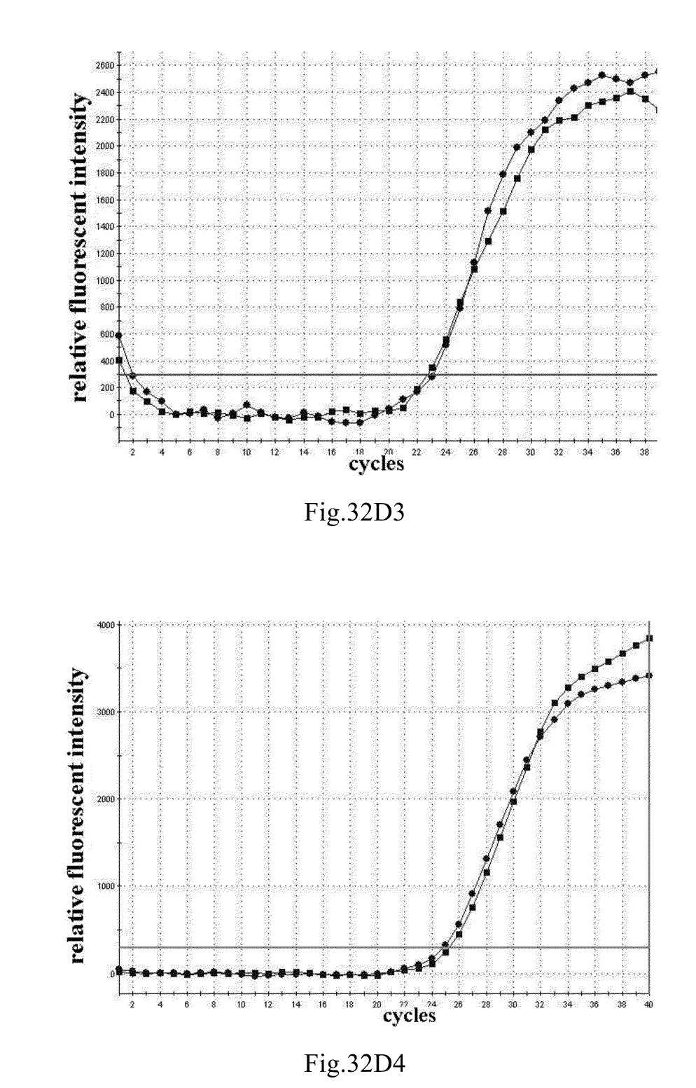

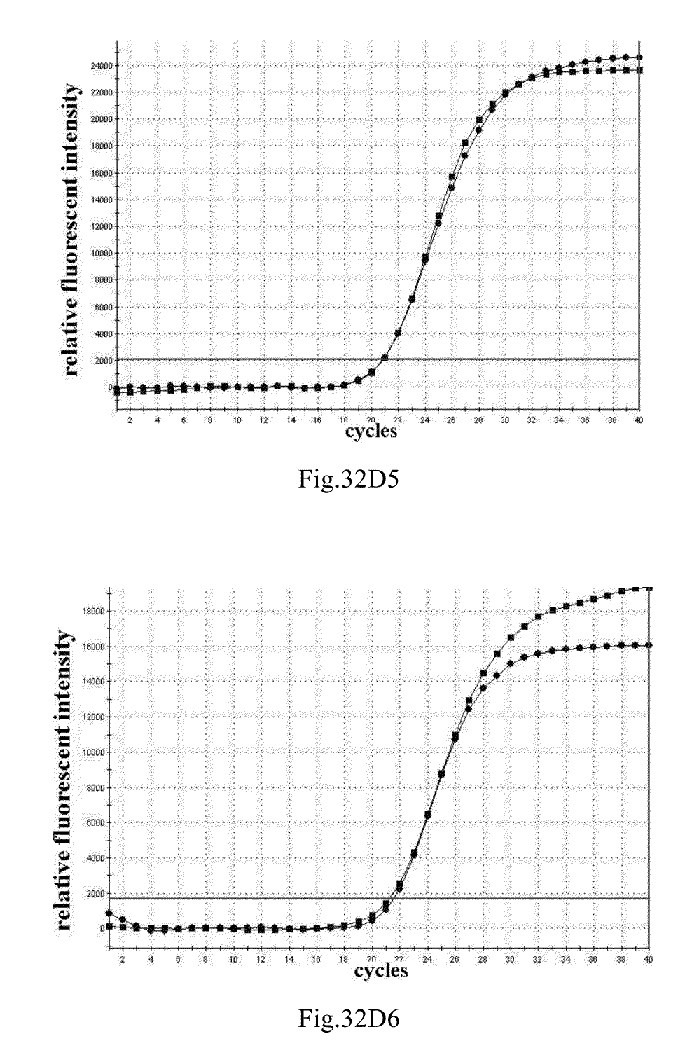

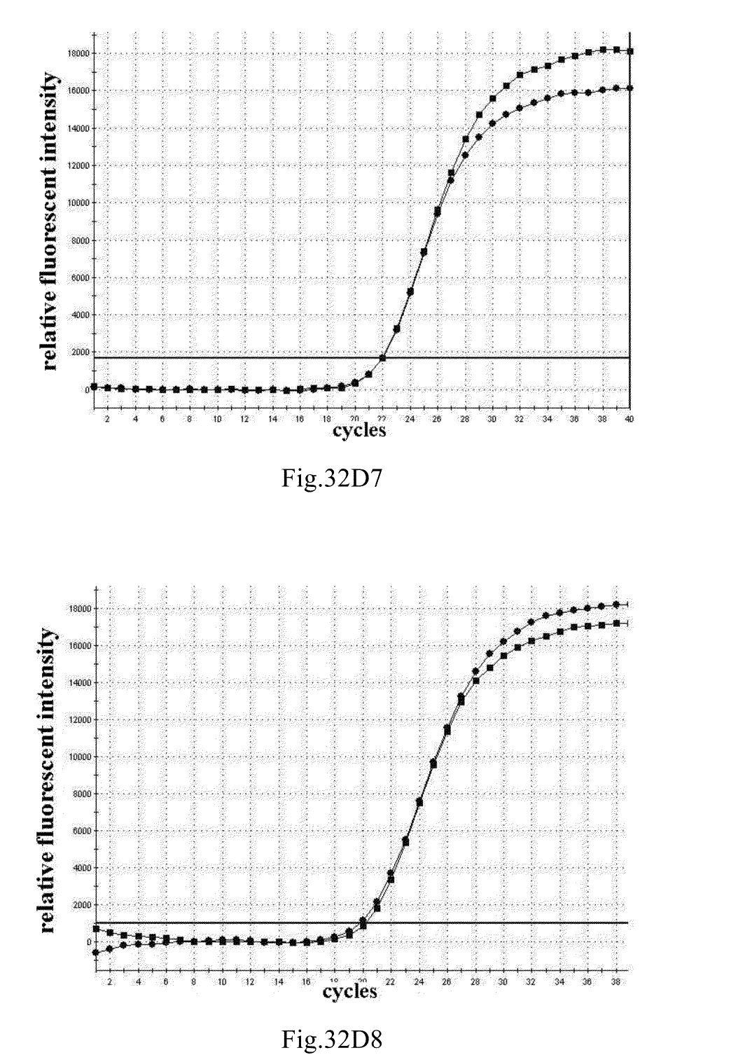

Based on the standard curve, the copy numbers of wild-type and mutant genome DNA can be calculated from the CT values of the sample. Then we obtain the ratio of mutant DNA to total DNA (wild-type plus all mutants at said site). As shown in FIG. 32, the wild-type CT value of EGFR Exon 21 in a sample is 19.15, whereas the value of position 2573 T→G mutant is 20.74. According to each corresponding standard curve formula (FIG. 31), we can calculate the copy number for each of them. We then obtain the ratio of the content of mutant to wild-type which was 89:100, and we estimate that about 47% EGFR gene in tissue sample has position 2573 T→G mutation.

### Example 26

### Use of Positive Plasmid Standard in Detecting Expression

1. Prepare plasmid standard with different concentration

Dilute the plasmid standard 10 times in turn to 5E-1 ng/μl, 5E-2 ng/μl, 5E-3 ng/μl, and 5E-4 ng/μl, as the template for fluorescent quantitative PCR.

2. Prepare cDNA of the sample using the method of Example 1.

3. The reaction system and condition for fluorescent quantitative PCR (Table 31, Table 32)

For detecting different genes, we should add corresponding primers (Table 12-28) and probes (Table 37) into the reaction system, wherein probes are labeled with fluorescence emitting groups which are selected from FAM, TET, HEX, or ROX, and fluorescence quenching groups which are selected from BHQ or TAMARA.

TABLE 37
Probes for detecting expression by
fluorescent quantitative PCR

| name | sequence | |
|---|---|---|
| ERCC1-P-1 | CCCGACTATGTGCTGGGCCA | ( SEQ ID NO: 171 ) |
| | GAG | |
| ERCC1-P-2 | CACAGGTGCTCTGGCCCAGC | ( SEQ ID NO: 172 ) |
| | ACATA | |
| RRM1-P-1 | CAGGATCGCTGTCTCTAACT | ( SEQ ID NO: 173 ) |
| | TGCACAA | |
| RRM1-P-2 | CAGCCAGGATCGCTGTCTCT | ( SEQ ID NO: 174 ) |
| | AACTTGCA | |
| BRCA1-P-1 | CCGTGCCAAAAGACTTCTAC | ( SEQ ID NO: 175 ) |
| | AGAGTGA | |
| BRCA1-P-2 | GCCAAAAGACTTCTACAGAG | ( SEQ ID NO: 176 ) |
| | TGA | |
| TUBB3-P-1 | CACAGGTGGCAAATATGTTC | ( SEQ ID NO: 177 ) |
| | CTCGT | |
| TUBB3-P-2 | CAGGTGGCAAATATGTTCCT | ( SEQ ID NO: 178 ) |
| ERBB3-P-1 | AAAGGTACTCCCTCCTCCCG | ( SEQ ID NO: 179 ) |
| | GGA | |
| ERBB3-P-2 | GGTACTCCCTCCTCCCGGGA | ( SEQ ID NO: 180 ) |
| TOP2A-P-1 | CTTCAGCACCATTTATCAGC | ( SEQ ID NO: 181 ) |
| | ACCATGG | |
| TOP2A-P-2 | TTCAGCACCATTTATCAGCA | ( SEQ ID NO: 182 ) |
| TYMS-P-1 | CGCGCTACAGCCTGAGAGAT | ( SEQ ID NO: 183 ) |
| | GAA | |
| TYMS-P-2 | CGCTACAGCCTGAGAGATG | ( SEQ ID NO: 184 ) |
| RAP-80-P-1 | CAGCCAGGAGGAGGAAGAA | ( SEQ ID NO: 185 ) |
| | GAGGA | |
| RAP-80-P-2 | GCCAGGAGGAGGAAGAAGA | ( SEQ ID NO: 186 ) |
| VEGFR1-P-1 | CTGCTGTCGCCCTGGTAGT | ( SEQ ID NO: 187 ) |
| | CATCAA | |
| VEGFR1-P-2 | CTGTCGCCCTGGTAGTCAT | ( SEQ ID NO: 188 ) |
| VEGFR2-P-1 | ACGGCGCTTGGACAGCATC | ( SEQ ID NO: 189 ) |
| | ACCAGT | |
| VEGFR2-P-2 | CGGCGCTTGGACAGCATCA | ( SEQ ID NO: 190 ) |
| | CC | |
| HER2-P-1 | CTCTCACACTGATAGACAC | ( SEQ ID NO: 191 ) |
| | CAACCGC | |
| HER2-P-2 | CGGTGTGAGAAGTGCAGCA | ( SEQ ID NO: 192 ) |
| | AGCCC | |
| EGFR-P-1 | CAATTCCACCGTGGCTTGC | ( SEQ ID NO: 193 ) |
| | ATTGA | |
| EGFR-P-2 | TCCACCGTGGCTTGCATTG | ( SEQ ID NO: 194 ) |
| | ATA | |
| VEGF-F-1 | CTCCACCATGCCAAGTGGT | ( SEQ ID NO: 195 ) |
| | CCCA | |
| VEGF-F-2 | CCACCATGCCAAGTGGTCC | ( SEQ ID NO: 196 ) |
| | C | |
| PPN-P-1 | CACCACAGAGGAGCAGGGC | ( SEQ ID NO: 197 ) |
| | TAC | |
| PPN-P-2 | AGCATGTCCTCCGGAAGCG | ( SEQ ID NO: 198 ) |
| | CC | |
| CCNB2-P-1 | AGAACCCTCAGCTCTGCAG | ( SEQ ID NO: 199 ) |
| | TGAC | |
| CCNB2-P-2 | ATTGGAAGTCATGCAGCAC | ( SEQ ID NO: 200 ) |
| | ATGGC | |
| ACTB-P-1 | CCCATCGAGCACGGCATCG | ( SEQ ID NO: 201 ) |
| | T | |
| ACTB-P-2 | ATGACGAGTCCGGCCCCTC | ( SEQ ID NO: 202 ) |
| | CATC | |
| 18S-P-1 | TGGTGTCGCGGAGCACGGA | ( SEQ ID NO: 203 ) |
| 18S-P-2 | CGCGCAAATTACCCACTCC | ( SEQ ID NO: 204 ) |
| | CGA | |

For detecting expressions, it needs to prepare two reaction systems: reaction system for gene of interest and reaction system for internal reference (ACTB or 18S rRNA). For example, for detecting the expression of ERCC1 gene, it needs to prepare ERCC1 detection system and ACTB or 18S rRNA detection system. For preparing ERCC1 detection system, the system needs to add ERCC1-F-1 (SEQ ID NO:57) or ERCC1-F-2 (SEQ ID NO:58) together with ERCC1-R-1 (SEQ ID NO:59) or ERCC1-R-2 (SEQ ID NO:60) primers, and ERCC1-P-1 (SEQ ID NO:171) or ERCC1-P-1 (SEQ ID NO:172) probe. For preparing ACTB detection system, the system needs to add ACTB-F-1 (SEQ ID NO:117) or ACTB-F-2 (SEQ ID NO:118) together with ACTB-R-1 (SEQ ID NO:119) or ACTB-R-2 (SEQ ID NO:120) primers, and ACTB-P-1 (SEQ ID NO:201) or ACTB-P-1 (SEQ ID NO:202) probe. For preparing 18S rRNA detection system, the system needs to add 18S-F-1 (SEQ ID NO:121) or 18S-F-2 (SEQ ID NO:122) together with 18S-R-1 (SEQ ID NO:123) or 18S-R-2 (SEQ ID NO:124) primers, and 18S-P-1 (SEQ ID NO:203) or 18S-P-1 (SEQ ID NO:204) probe.

4. Draw the standard curve

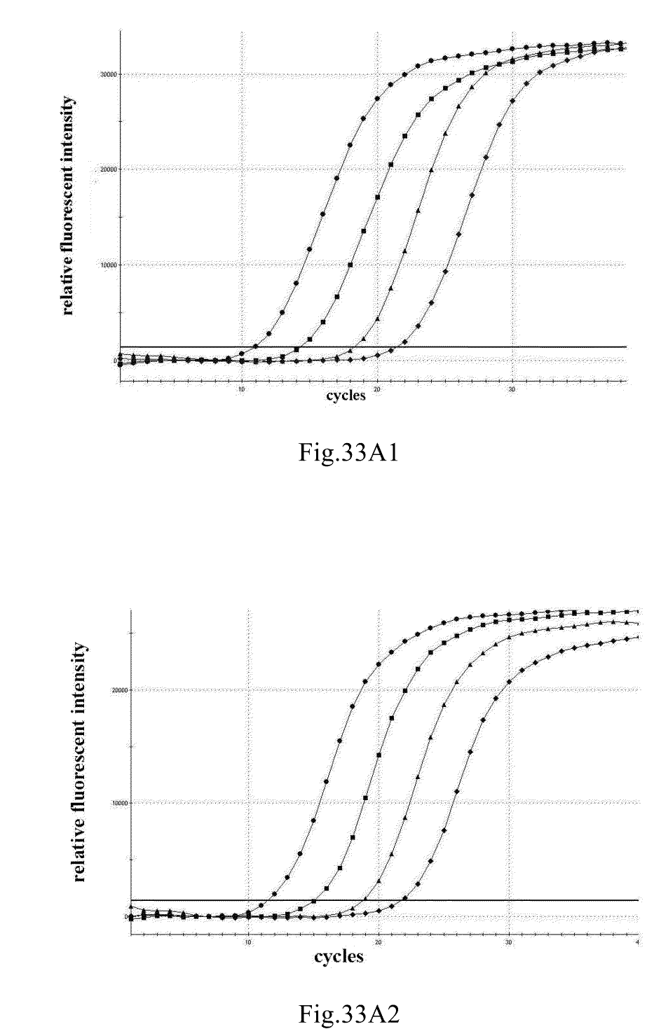

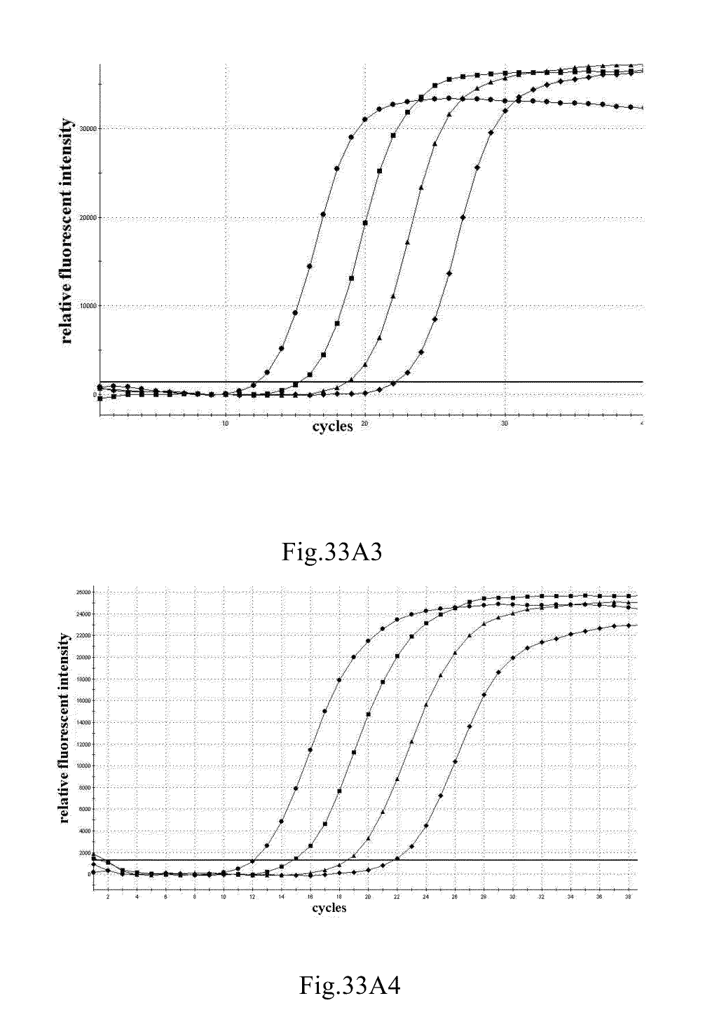

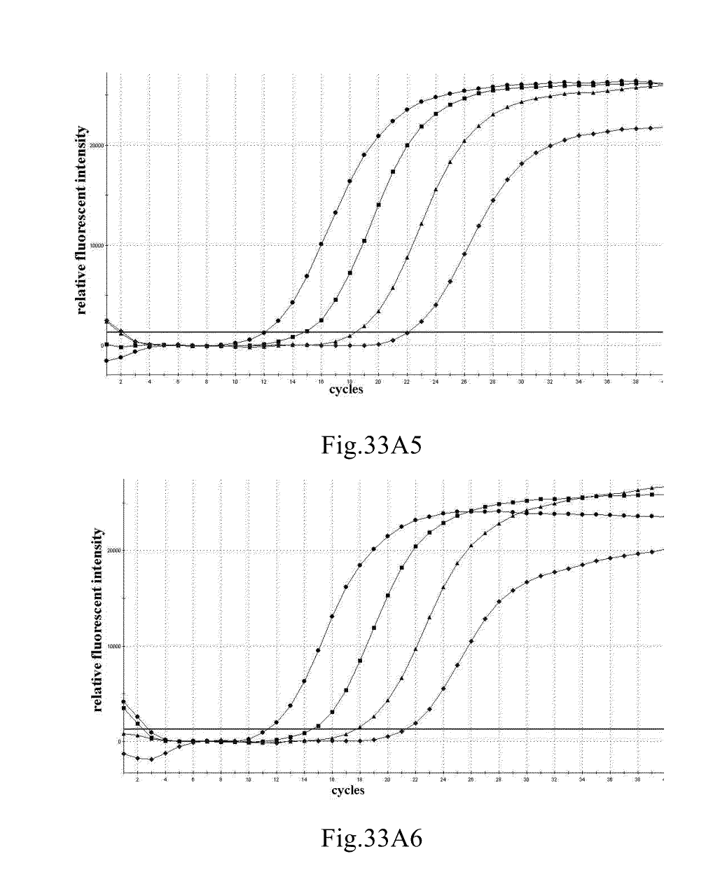

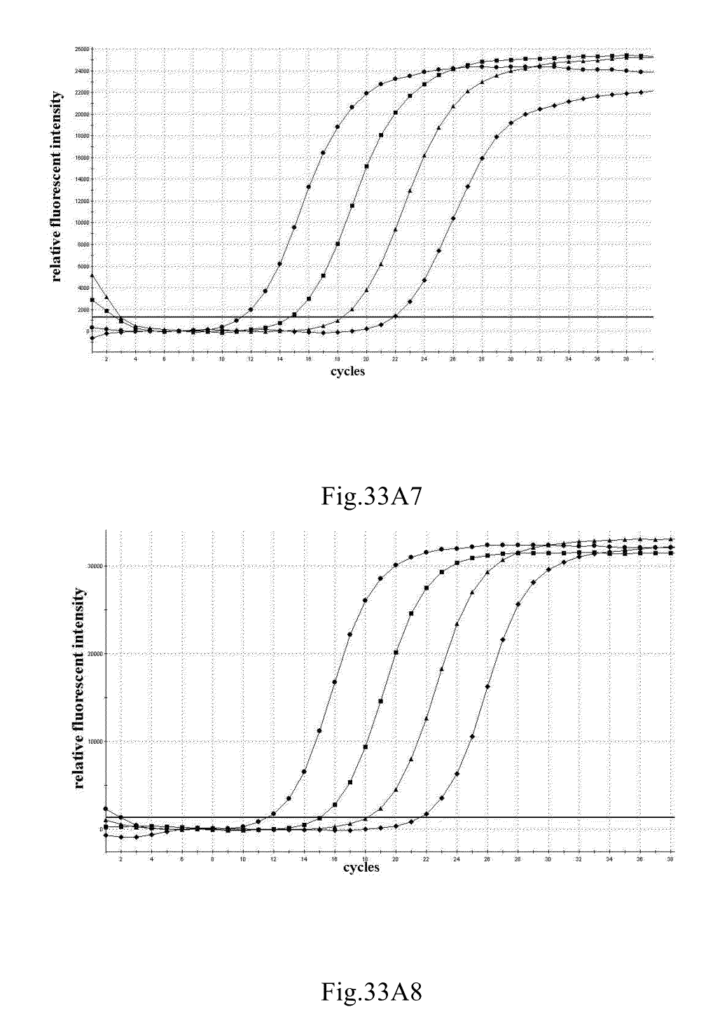

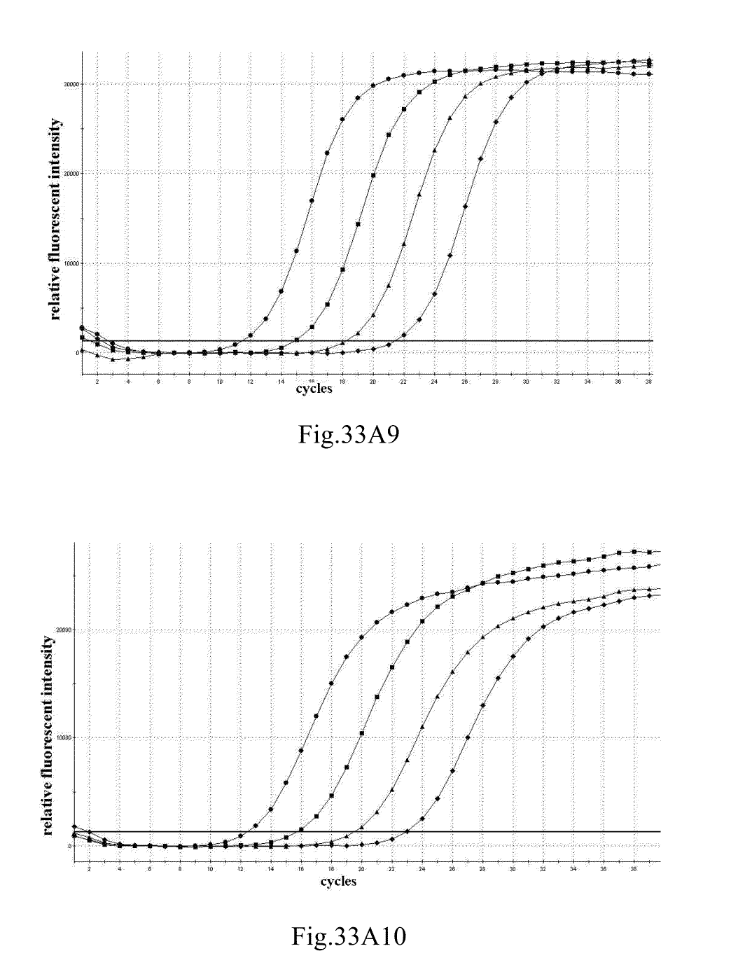

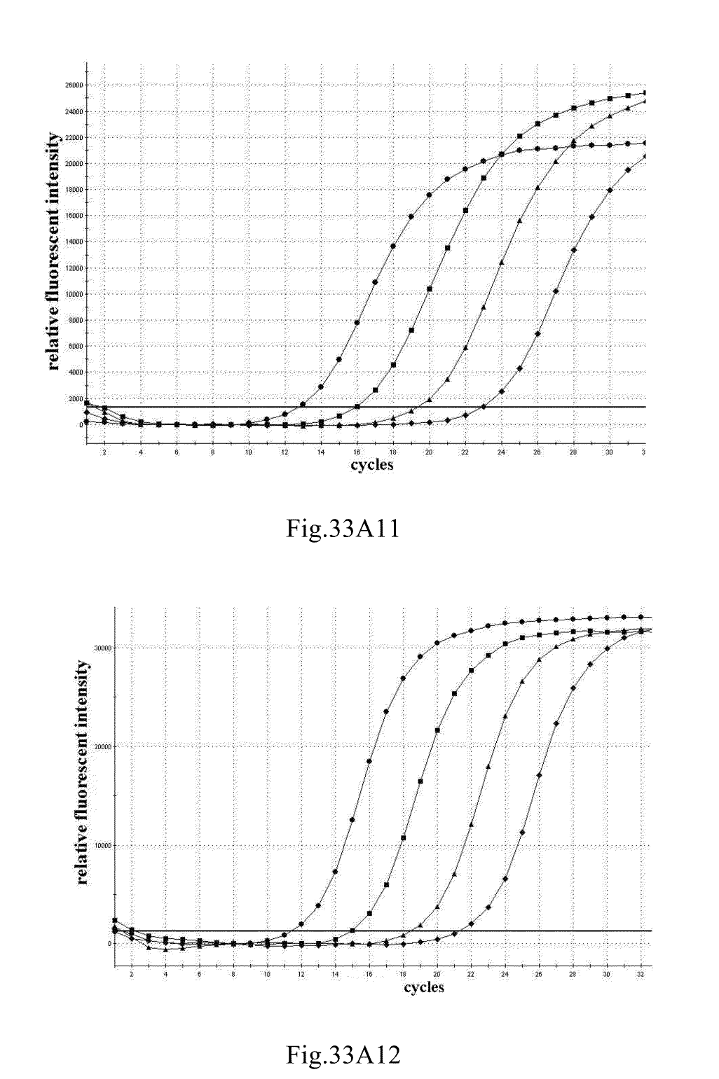

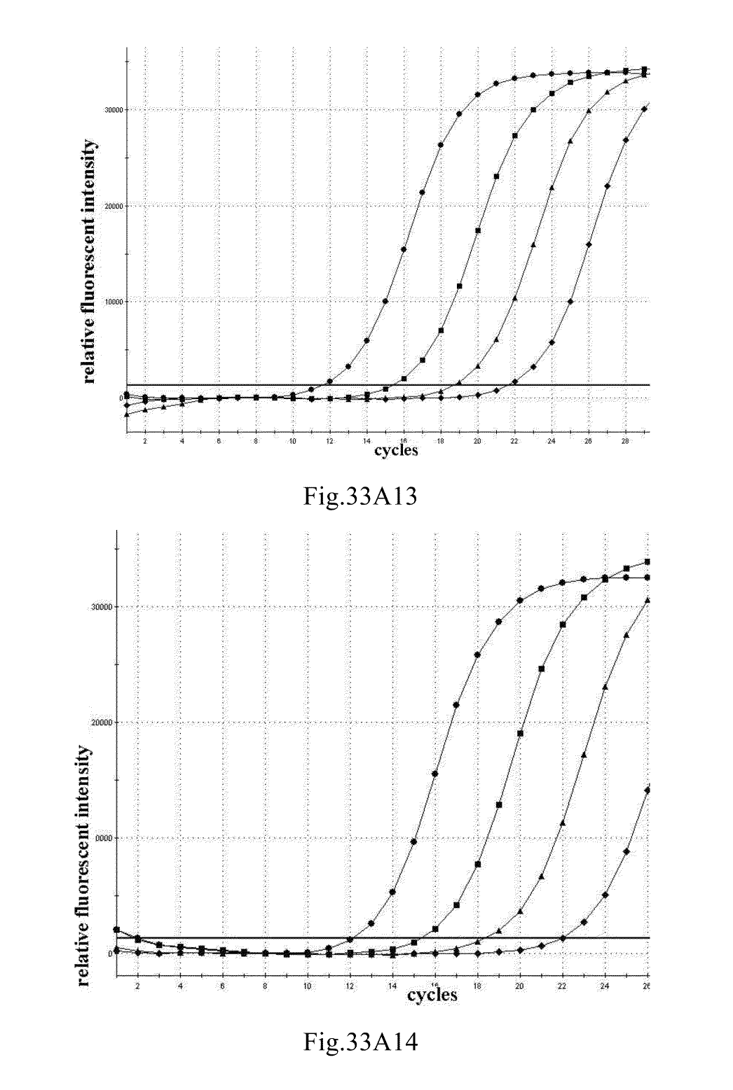

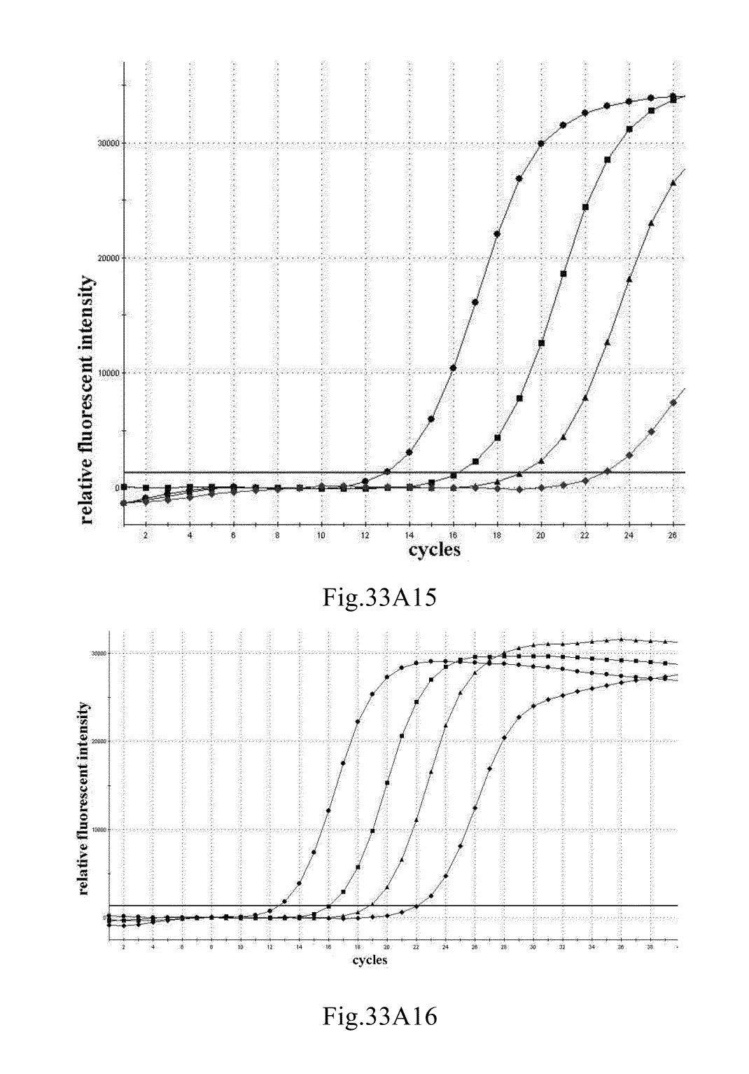

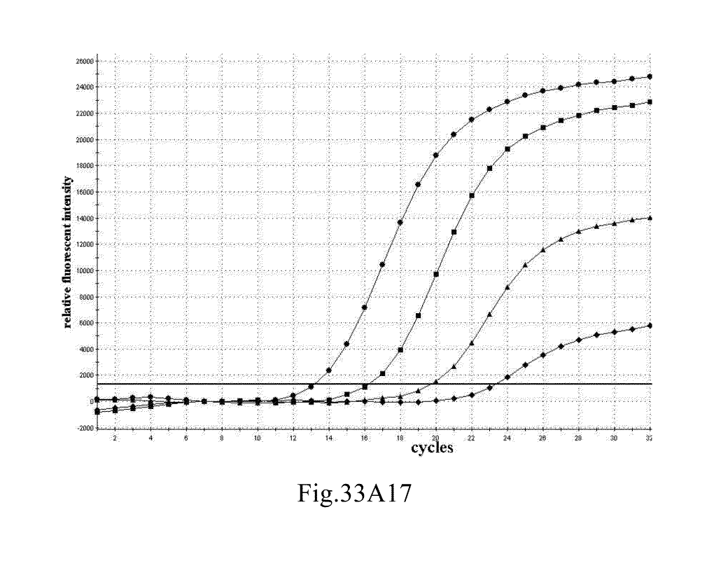

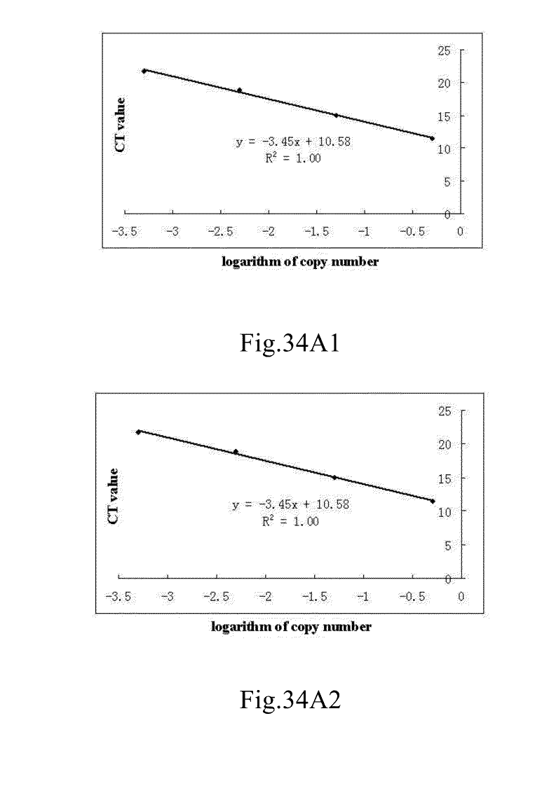

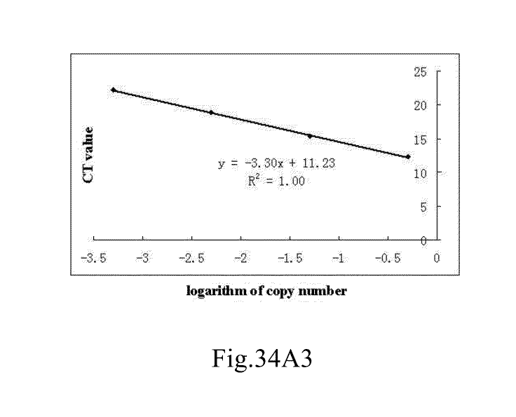

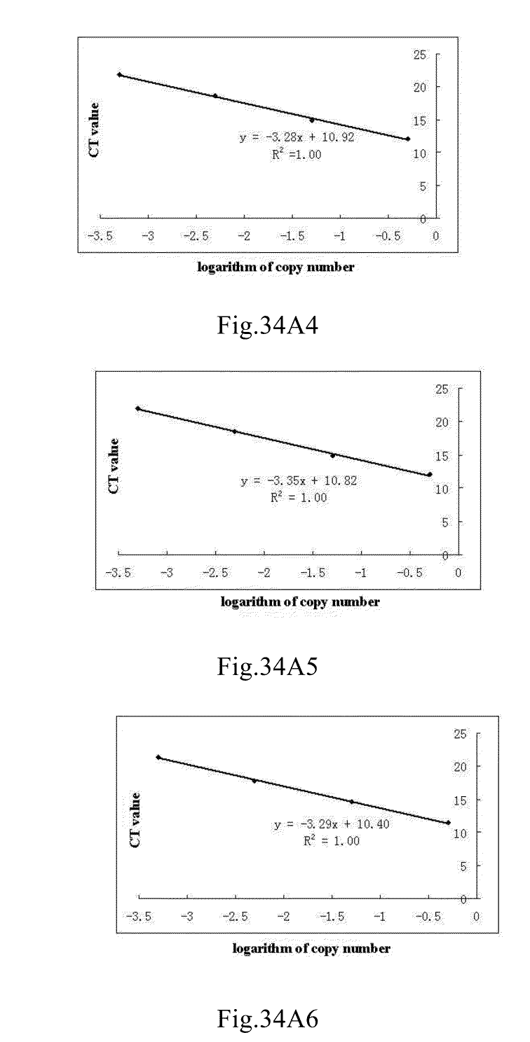

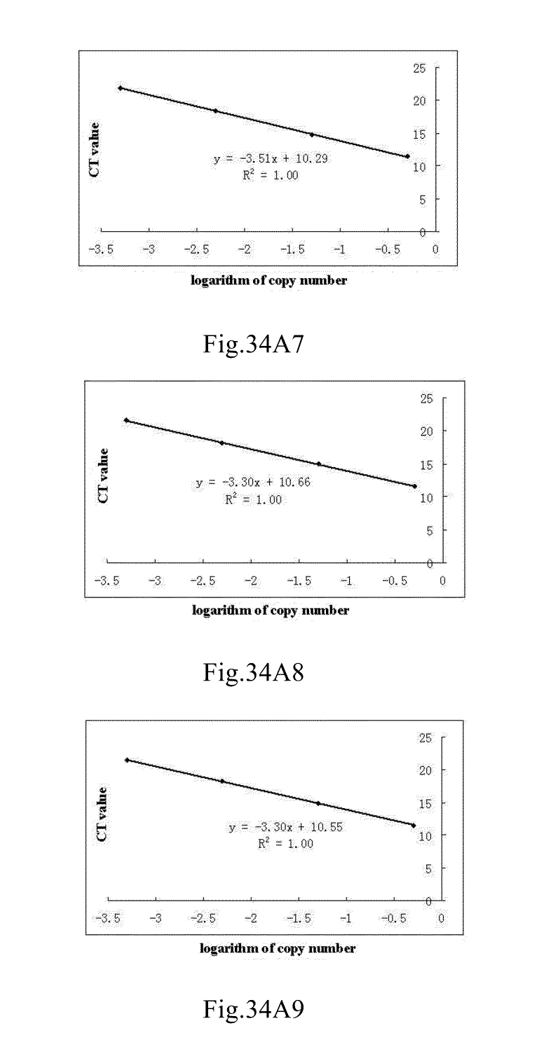

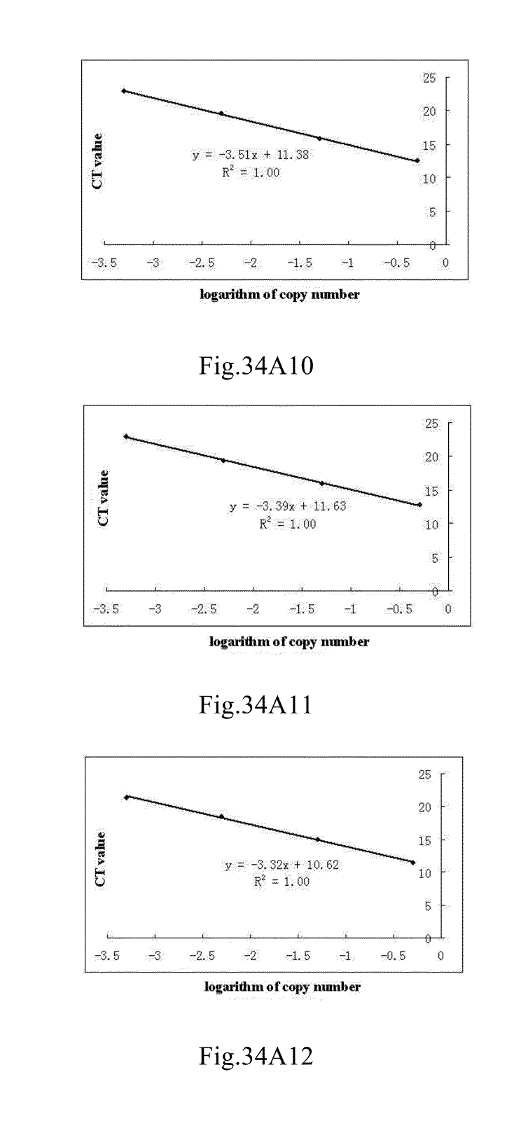

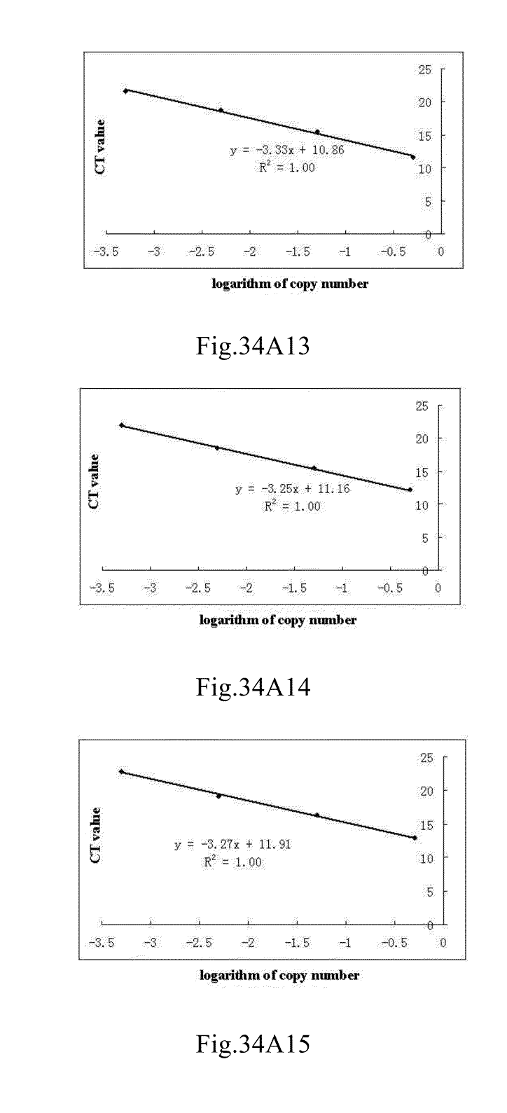

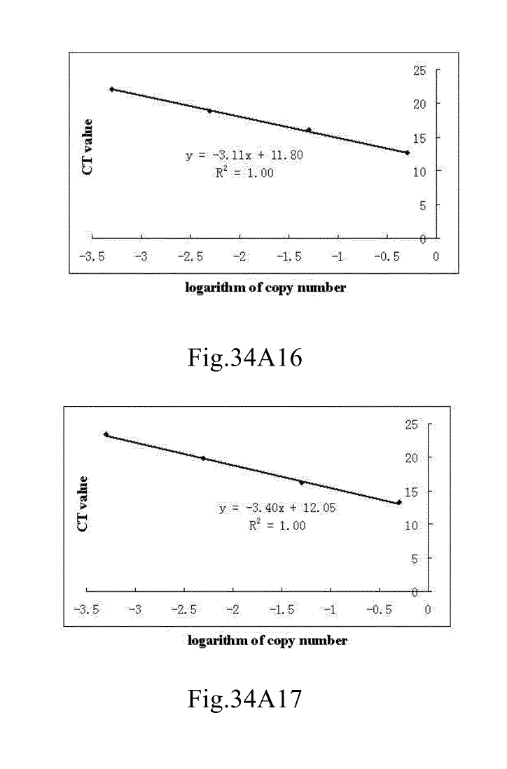

The standard curve is drawn based on the CT values obtained from the standard in Step 3. FIG. 33 shows the amplification curve of plasmid standard, in which the five rising curves represent, from left to right, the amplification curves of the plasmid standards 10 times diluted in turn. The horizontal axis represents cycle number, and the vertical axis represents fluorescent detection value. Accordingly, it is possible to draw the standard curve for calculation (FIG. 34). In FIG. 34, the horizontal axis represents the logarithm of copy number of the template, the vertical axis represents CT value, wherein the copy number of template=mass/(molecular weight)×6.02×10²³. In the present experiment, the plasmid consists of pMD18-T vector and an insert. All of the inserts have a length of about 100 bp, very short compared with that of pMD18-T vector. Therefore, they can be ignored. The copy number of 0.5 ng/μl plasmid is 10¹⁰.

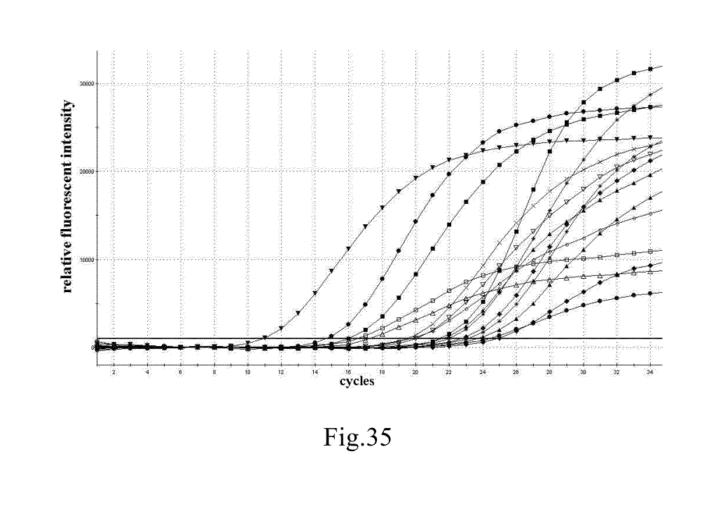

5. Calculate the gene expression in a sample using standard curves Based on the standard curve, the copy numbers of gene of interest and internal reference gene can be calculated from the CT values of the sample. The ratio of copy numbers reflects the expression of gene of interest against internal reference gene. As shown in FIG. 35, the CT value of ERCC1 gene in a tissue sample is 14.98, whereas the CT value of ACTB gene is 15.88. According to each corresponding standard curve formula (FIG. 34), we can calculate the copy number for each of them. We then calculate out that the expression amount of ERCC1 is 0.47. The copy numbers and expression amount of other genes can be obtained similarly.

### Example 27

### Use of Positive Plasmid Standard in Detecting Genome DNA Gene Amplification

1. Prepare plasmid standard with different concentration

Dilute the plasmid standard 10 times in turn to 5E-1 ng/μl, 5E-2 ng/μl, 5E-3 ng/μl, and 5E-4 ng/μl, as the template for fluorescent quantitative PCR.

2. Prepare DNA of the sample using the method of Example 1.

3. The reaction system and condition for fluorescent quantitative PCR (Table 31, Table 32)

For detecting different genes, we should add corresponding primers (Table 29 and Table 30) and probes (Table 38) into the reaction system, wherein probes are labeled with fluorescence emitting groups which are selected from FAM, TET, HEX, or ROX, and fluorescence quenching groups which are selected from BHQ or TAMARA.

TABLE 38
Probes for detecting gene amplification by
fluorescent quantitative PCR

| name | sequence | |
|---|---|---|
| HER2-P-1 | CTCCTCCACTCACTAGCACAA | (SEQ ID NO: 205) |
| | TGAC | |
| HER2-P-2 | TCAAGGCTCAAGGTTCCTCTT | (SEQ ID NO: 206) |
| | CTGC | |
| ACTB-P-1 | TGGCACCCAGCACAATGAAG | (SEQ ID NO: 207) |
| | ATCA | |
| ACTB-P-2 | CCCATCGAGCACGGCATCGT | (SEQ ID NO: 208) |

For detecting gene amplication, it needs to prepare two reaction systems: reaction system for gene of interest and reaction system for internal reference (ACTB). HER2 amplification system needs to add O-HER2-F-1 (SEQ ID NO:125) or O-HER2-F-2 (SEQ ID NO:126) together with O-HER2-R-1 (SEQ ID NO:127) or O-HER2-R-2 (SEQ ID NO:128) primers, and HER2-P-1 (SEQ ID NO:205) or HER2-P-2 (SEQ ID NO:206) probe. ACTB amplification system needs to add ACTB-F-1 (SEQ ID NO:129) or ACTB-F-2 (SEQ ID NO:130) together with ACTB-R-1 (SEQ ID NO:131) or ACTB-R-2 (SEQ ID NO:132) and ACTB-P-1 (SEQ ID NO:207) or ACTB-P-2 (SEQ ID NO:208) probe.

4. Draw the standard curve

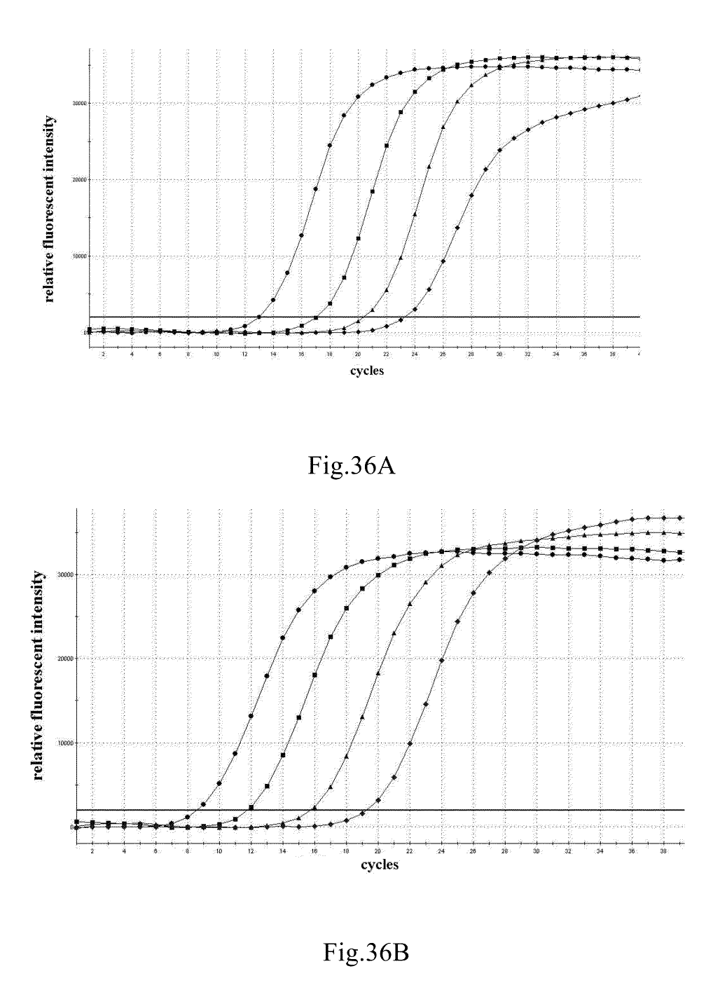

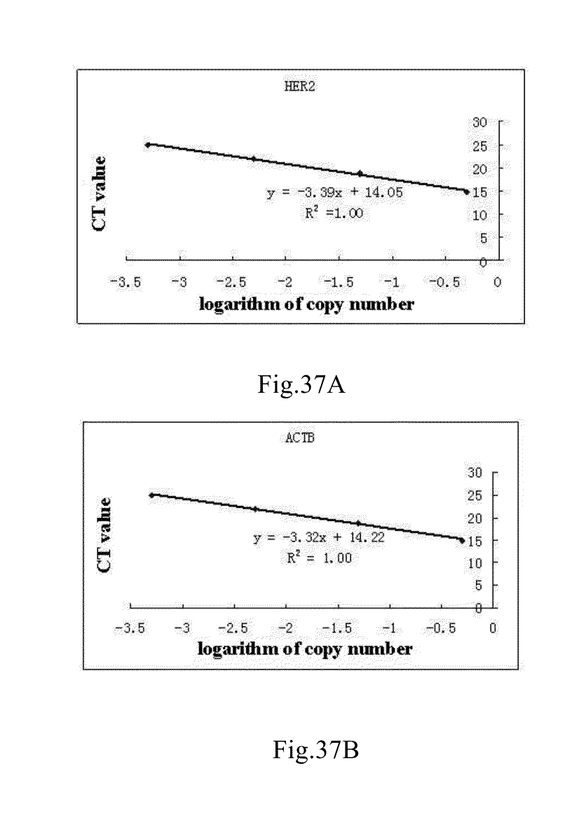

The standard curve is drawn based on the CT values obtained from the standard in Step 3. FIG. 36 shows the amplification curve of plasmid standard, in which the five rising curves represent, from left to right, the amplification curves of the plasmid standards 10 times diluted in turn which are 5E-1 ng/μl, 5E-2 ng/μl, 5E-3 ng/μl, 5E-4 ng/μl, and 5E-5 ng/μl. The horizontal axis represents cycle number, and the vertical axis represents fluorescent detection value. Accordingly, it is possible to draw the standard curve for calculation (FIG. 37). In FIG. 37, the horizontal axis represents the logarithm of copy number of the template, the vertical axis represents CT value, wherein the copy number of template=mass/(molecular weight)×6.02×10²³. In the present experiment, the plasmid consists of pMD18-T vector and an insert. Because the lengths of the inserts are almost identical, the biggest difference only lies in twenties bases, which can be ignored with respect to the length of 2692 bp for PMD18-T vector. Therefore, the ratio of copies of HER2 to ACTB plasmid standard≈the ratio of mass.

5. Calculate HER2 gene amplification in a sample

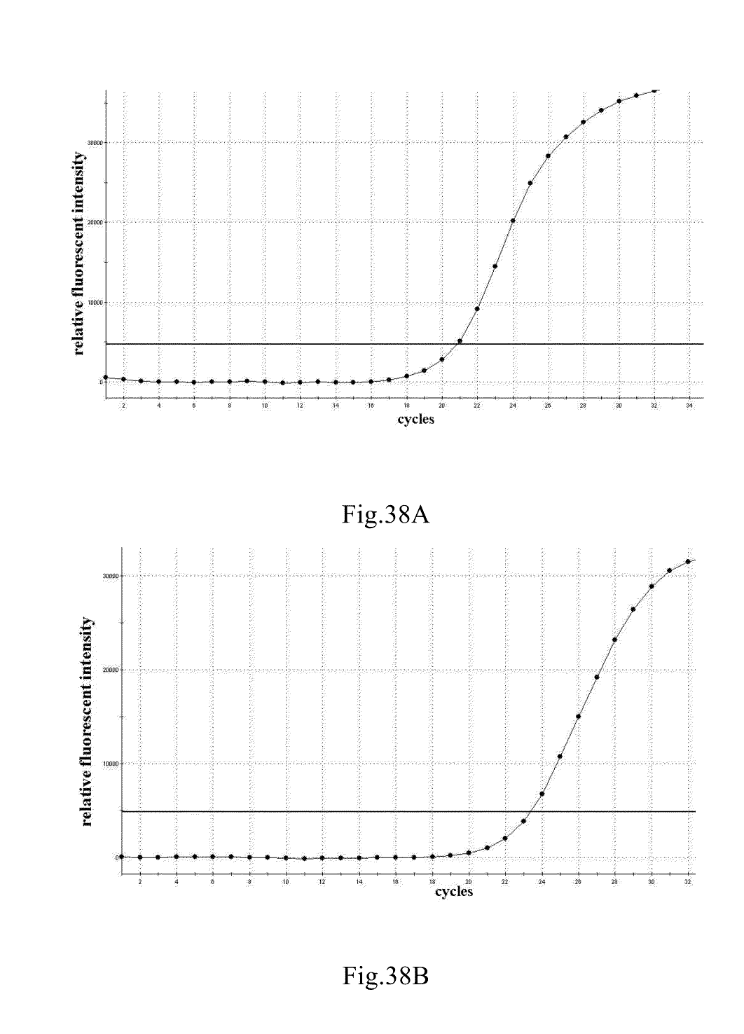

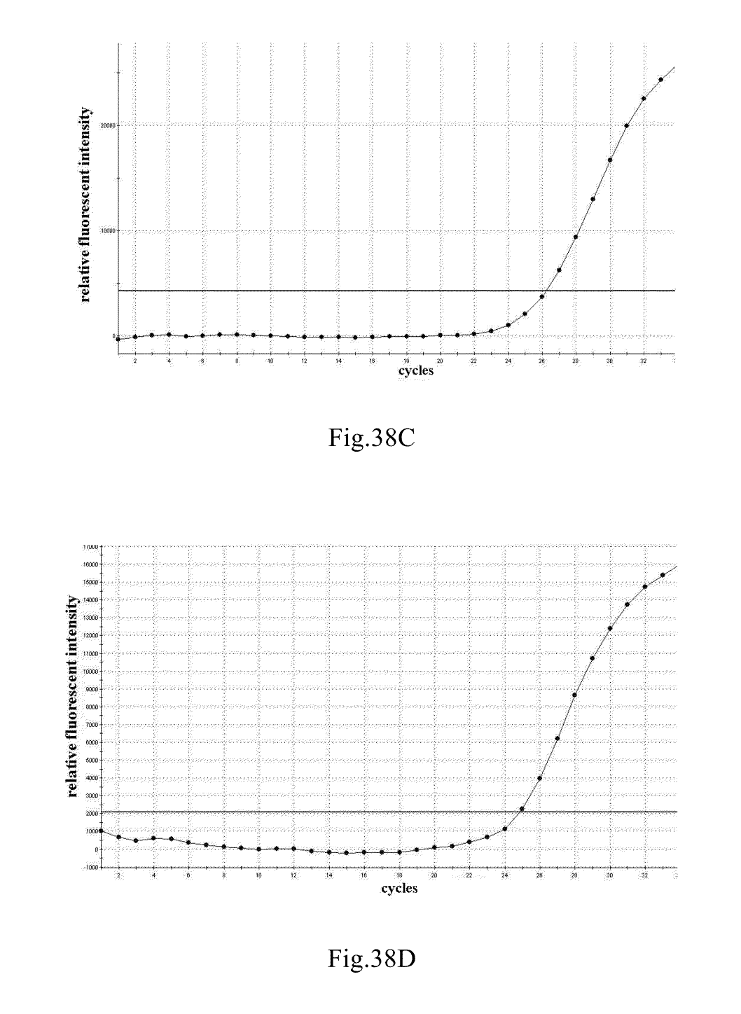

Based on the standard curve, the copy numbers of HER2 and ACTB genome DNA can be calculated from the CT values of the sample. The ratio of HER2 DNA to ACTB DNA reflects HER2 gene amplification amount. As shown in FIG. 38, the CT values of HER2 in paraffin embedded tissue sample and fresh tissue are 21.05 and 23.40 respectively, whereas the CT value of internal reference genes are 25.88 and 24.95 respectively. According to each corresponding standard curve formula (FIG. 37), we can calculate the copy number for each of them. We then calculate out that the gene amplification amounts in the tested samples are 281% and 161% respectively.

We claim:

1. A plasmid standard for detection by fluorescent quantitative PCR, characterized in that the vector for said plasmid is pMD18-T, and the insert for said plasmid is any one of following (1)-(4) sequence to be detected, which is for detecting the mutation of corresponding gene,
(1) EGFR Exon 18, 19, 21;
(2) KRAS Codon 12, 13;
(3) BCRP position 482 amino acid; or
(4) BRAF position 600 amino acid.

2. A plasmid standard according to claim 1, characterized in that the insert for said plasmid is:
(1) SEQ ID NO: 209, SEQ ID NO: 210, SEQ ID NO: 211, SEQ ID NO: 212, SEQ ID NO: 213, SEQ ID NO: 214, SEQ ID NO: 215, SEQ ID NO: 216;
(2) SEQ ID NO: 217, SEQ ID NO: 218, SEQ ID NO: 219, SEQ ID NO: 220, SEQ ID NO: 221, SEQ ID NO: 222;
(3) SEQ ID NO: 223, SEQ ID NO: 224, SEQ ID NO: 225; or
(4) SEQ ID NO: 226, SEQ ID NO: 227.

3. A plasmid standard according to claim 1, characterized in that the primer for preparing and detecting said plasmid is:
(1) SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23;
(2) SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 38;
(3) SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49; or
(4) SEQ ID NO: 51, SEQ ID NO: 52, SEQ ID NO: 53, SEQ ID NO: 54, SEQ ID NO: 55, SEQ ID NO: 56.

4. A plasmid standard according to claim 1, characterized in that the probe for detecting said plasmid is:
(1) SEQ ID NO: 133, SEQ ID NO: 134, SEQ ID NO: 135, SEQ ID NO: 136, SEQ ID NO: 137, SEQ ID NO: 138, SEQ ID NO: 139, SEQ ID NO: 140, SEQ ID NO: 141, SEQ ID NO: 142, SEQ ID NO: 143, SEQ ID NO: 144, SEQ ID NO: 145, SEQ ID NO: 146, SEQ ID NO: 147, SEQ ID NO: 148;
(2) SEQ ID NO: 149, SEQ ID NO: 150, SEQ ID NO: 151, SEQ ID NO: 152, SEQ ID NO: 153, SEQ ID NO: 154, SEQ ID NO: 155, SEQ ID NO: 156, SEQ ID NO: 157, SEQ ID NO: 158, SEQ ID NO: 159, SEQ ID NO: 160;
(3) SEQ ID NO: 161, SEQ ID NO: 162, SEQ ID NO: 163, SEQ ID NO: 164, SEQ ID NO: 165, SEQ ID NO: 166; or
(4) SEQ ID NO: 167, SEQ ID NO: 168, SEQ ID NO: 169, SEQ ID NO: 170.

5. A plasmid standard for detection by fluorescent quantitative PCR, characterized in that the vector for said plasmid is pMD18-T, and the insert for said plasmid is any one of following (1)-(17) sequence to be detected, which is for detecting the expression of corresponding gene:
(1) ERCC1 mRNA;
(2) RRM1 mRNA;
(3) BRCA1 mRNA;
(4) TUBB3 mRNA;
(5) ERBB3 mRNA;
(6) TOP2A mRNA;
(7) TYMS mRNA;
(8) RAP-80 mRNA;
(9) VEGFR1 mRNA;
(10) VEGFR2 mRNA;
(11) HER2 mRNA;
(12) EGFR mRNA;
(13) VEGF mRNA;
(14) PPN mRNA;
(15) CCNB2 mRNA;
(16) ACTB mRNA; or
(17) 18S rRNA.

6. A plasmid standard according to claim 3, characterized in that the insert for said plasmid is:
(1) SEQ ID NO: 228;
(2) SEQ ID NO: 229;
(3) SEQ ID NO: 230;
(4) SEQ ID NO: 231;
(5) SEQ ID NO: 232;
(6) SEQ ID NO: 233;
(7) SEQ ID NO: 234;
(8) SEQ ID NO: 235;
(9) SEQ ID NO: 236;
(10) SEQ ID NO: 237;
(11) SEQ ID NO: 238;
(12) SEQ ID NO: 239;
(13) SEQ ID NO: 240;
(14) SEQ ID NO: 241;
(15) SEQ ID NO: 242;
(16) SEQ ID NO: 243; or
(17) SEQ ID NO: 244.

7. A plasmid standard according to claim 1, characterized in that the primer for preparing and detecting said plasmid is:
(1) SEQ ID NO: 57, SEQ ID NO: 58, SEQ ID NO: 59, SEQ ID NO: 60;
(2) SEQ ID NO: 61, SEQ ID NO: 62, SEQ ID NO: 63, SEQ ID NO: 64;
(3) SEQ ID NO: 65, SEQ ID NO: 66, SEQ ID NO: 67, SEQ ID NO: 68;
(4) SEQ ID NO: 69, SEQ ID NO: 70, SEQ ID NO: 71, SEQ ID NO: 72;
(5) SEQ ID NO: 73, SEQ ID NO: 74, SEQ ID NO: 75, SEQ ID NO: 76;
(6) SEQ ID NO: 77, SEQ ID NO: 78, SEQ ID NO: 79, SEQ ID NO: 80;
(7) SEQ ID NO: 81, SEQ ID NO: 82, SEQ ID NO: 83, SEQ ID NO: 84;
(8) SEQ ID NO: 85, SEQ ID NO: 86, SEQ ID NO: 87, SEQ ID NO: 88;
(9) SEQ ID NO: 89, SEQ ID NO: 90, SEQ ID NO: 91, SEQ ID NO: 92;
(10) SEQ ID NO: 93, SEQ ID NO: 94, SEQ ID NO: 95, SEQ ID NO: 96;
(11) SEQ ID NO: 97, SEQ ID NO: 98, SEQ ID NO: 99, SEQ ID NO: 100;
(12) SEQ ID NO: 101, SEQ ID NO: 102, SEQ ID NO: 103, SEQ ID NO: 104;
(13) SEQ ID NO: 105, SEQ ID NO: 106, SEQ ID NO: 107, SEQ ID NO: 108;
(14) SEQ ID NO: 109, SEQ ID NO: 110, SEQ ID NO: 111, SEQ ID NO: 112;
(15) SEQ ID NO: 113, SEQ ID NO: 114, SEQ ID NO: 115, SEQ ID NO:116;
(16) SEQ ID NO: 117, SEQ ID NO: 118, SEQ ID NO: 119, SEQ ID NO: 120; or
(17) SEQ ID NO: 121, SEQ ID NO: 122, SEQ ID NO: 123, SEQ ID NO: 124.

8. A plasmid standard according to claim 1, characterized in that the probe for detecting said plasmid is:
(1) SEQ ID NO: 171, SEQ ID NO: 172;
(2) SEQ ID NO: 173, SEQ ID NO: 174;
(3) SEQ ID NO: 175, SEQ ID NO: 176;
(4) SEQ ID NO: 177, SEQ ID NO: 178;
(5) SEQ ID NO: 179, SEQ ID NO: 180;
(6) SEQ ID NO: 181, SEQ ID NO: 182;
(7) SEQ ID NO: 183, SEQ ID NO: 184;
(8) SEQ ID NO: 185, SEQ ID NO: 186;
(9) SEQ ID NO: 187, SEQ ID NO: 188;
(10) SEQ ID NO: 189, SEQ ID NO: 190;
(11) SEQ ID NO: 191, SEQ ID NO: 192;
(12) SEQ ID NO: 193, SEQ ID NO: 194;
(13) SEQ ID NO: 195, SEQ ID NO: 196;
(14) SEQ ID NO: 197, SEQ ID NO: 198;
(15) SEQ ID NO: 199, SEQ ID NO: 200;
(16) SEQ ID NO: 201, SEQ ID NO: 202; or
(17) SEQ ID NO: 203, SEQ ID NO: 204.

9. A plasmid standard for detection by fluorescent quantitative PCR, characterized in that the vector for said plasmid is pMD18-T, and the insert for said plasmid is any one of following (1)-(17) sequence to be detected, which is for detecting the amplification of corresponding gene:
(1) HER2 genome DNA; or
(2) ACTB genome DNA.

10. A plasmid standard according to claim 5, characterized in that the insert for said plasmid is:
(1) SEQ ID NO: 245; or
(2) SEQ ID NO: 246.

11. A plasmid standard according to claim 1, characterized in that the primer for preparing and detecting said plasmid is:
(1) SEQ ID NO:125, SEQ ID NO:126, SEQ ID NO:127, SEQ ID NO:128; or
(2) SEQ ID NO:129, SEQ ID NO:130, SEQ ID NO:131, SEQ ID NO:132.

12. A plasmid standard according to claim 1, characterized in that the probe for detecting said plasmid is:
(1) SEQ ID NO: 205, SEQ ID NO: 206; or
(2) SEQ ID NO: 207, SEQ ID NO: 208.
